(19) United States
(12) Patent Application Publication
(10) Pub. No.: US 2024/0352625 A1
(43) Pub. Date: Oct. 24, 2024

(54) LIBRARIES OF GENETIC PACKAGES COMPRISING NOVEL HC CDR3 DESIGNS

(71) Applicant: Takeda Pharmaceutical Company Limited, Osaka (JP)

(72) Inventor: Robert Charles Ladner, Ijamsville, MD (US)

(73) Assignee: Takeda Pharmaceutical Company Limited, Osaka (JP)

(21) Appl. No.: 18/421,319

(22) Filed: Jan. 24, 2024

Related U.S. Application Data
(62) Division of application No. 16/897,069, filed on Jun. 9, 2020, now Pat. No. 11,926,926.
Application No. 16/897,069 is a continuation of application No. 15/836,230, filed on Dec. 8, 2017, now Pat. No. 10,718,066.
Application No. 15/836,230 is a division of application No. 12/922,153, filed on Jan. 24, 2011, now Pat. No. 9,873,957.
(60) Provisional application No. 61/047,529, filed on Apr. 24, 2008.
(60) Provisional application No. 61/036,219, filed on Mar. 13, 2008.

(51) Int. Cl. C40B 50/06; C07K 16/00; C07K 16/12; C07K 16/28; C40B 40/08

(52) U.S. Cl. CPC C40B 50/06; C07K 16/005; C07K 16/1275; C07K 16/28; C40B 40/08; C07K 2317/565

(57) ABSTRACT

Provided are compositions and methods for preparing and identifying antibodies having CDR3s that vary in sequence and in length from very short to very long which in certain embodiments may bind to a carbohydrate moiety or the active site of an enzyme. Libraries coding for antibodies with the CDR3s are also provided. The libraries can be provided by modifying a pre-existing nucleic acid library.

### CROSS REFERENCE TO RELATED APPLICATIONS

This application is a continuation of U.S. application Ser. No. 16/897,069, filed on Jun. 9, 2020, which is a continuation of U.S. application Ser. No. 15/836,230, filed on Dec. 8, 2017 and issued as U.S. Pat. No. 10,718,066, which is a divisional of U.S. application Ser. No. 12/922,153, filed on Jan. 24, 2011 and issued as U.S. Pat. No. 9,873,957, which is a National Stage Filing under 35 U.S.C. 371 of International Application No. PCT/US2009/037174, filed on Mar. 13, 2009, which claims priority to U.S. Application Ser. No. 61/036,219, filed on Mar. 13, 2008 and to U.S. Application Ser. No. 61/047,529, filed on Apr. 24, 2008. The disclosures of the prior applications are considered part of (and are incorporated by reference in) the disclosure of this application.

### REFERENCE TO AN ELECTRONIC SEQUENCE LISTING

The contents of the electronic sequence listing (D061770048US04-SEQ-KVC.xml; Size: 1,037,453 bytes; and Date of Creation: Jan. 22, 2024) is herein incorporated by reference in its entirety.

### BACKGROUND

It is now common practice in the art to prepare libraries of genetic packages that individually display, display and express, or comprise a member of a diverse family of peptides, polypeptides or proteins and collectively display, display and express, or comprise at least a portion of the amino acid diversity of the family. In many common libraries, the peptides, polypeptides or proteins are related to antibodies (e.g., single chain Fv (scFv), Fv, Fab, whole antibodies or minibodies (i.e., dimers that consist of V_{H} linked to V_{L})). Often, they comprise one or more of the CDRs and framework regions of the heavy and light chains of human antibodies.

Peptide, polypeptide or protein libraries have been produced in several ways. Sec, e.g., Knappik et al., J. Mol. Biol., 296, pp. 57-86 (2000), which is incorporated herein by reference. One method is to capture the diversity of native donors, either naive or immunized. Another way is to generate libraries having synthetic diversity. A third method is a combination of the first two. Typically, the diversity produced by these methods is limited to sequence diversity, i.e., each member of the library has the same length but differs from the other members of the family by having different amino acids or variegation at a given position in the peptide, polypeptide or protein chain. Naturally diverse peptides, polypeptides or proteins, however, are not limited to diversity only in their amino acid sequences. For example, human antibodies are not limited to sequence diversity in their amino acids, they are also diverse in the lengths of their amino acid chains.

For antibodies, diversity in length occurs, for example, during variable region rearrangements. See e.g., Corbett et al., J. Mol. Biol., 270, pp. 587-97 (1997). The joining of V genes to J genes, for example, results in the inclusion of a recognizable D segment in CDR3 in about half of the heavy chain antibody sequences, thus creating regions encoding varying lengths of amino acids. D segments are more common in antibodies having long HC CDR3s. The following also may occur during joining of antibody gene segments: (i) the end of the V gene may have zero to several bases deleted or changed; (ii) the end of the D segment may have zero to many bases removed or changed; (iii) a number of random bases may be inserted between V and D or between D and J; and (iv) the 5′ end of J may be edited to remove or to change several bases. These rearrangements result in antibodies that are diverse both in amino acid sequence and in length.

Libraries that contain only amino acid sequence diversity are, thus, disadvantaged in that they do not reflect the natural diversity of the peptide, polypeptide or protein that the library is intended to mimic. Further, diversity in length may be important to the ultimate functioning of the protein, peptide or polypeptide. For example, with regard to a library comprising antibody regions, many of the peptides, polypeptides, proteins displayed, displayed and expressed, or comprised by the genetic packages of the library may not fold properly or their binding to an antigen may be disadvantaged, if diversity both in sequence and length are not represented in the library.

An additional disadvantage of such libraries of genetic packages that display, display and express, or comprise peptides, polypeptides and proteins is that they are not focused on those members that are based on natural occurring diversity and thus on members that are most likely to be functional and least likely to be immunogenic. Rather, the libraries, typically, attempt to include as much diversity or variegation as possible at every amino acid residue. This makes library construction time-consuming and less efficient than necessary. The large number of members that are produced by trying to capture complete diversity also makes screening more cumbersome than it needs to be. This is particularly true given that many members of the library will not be functional.

In addition to the labor of constructing synthetic libraries is the question of immunogenicity. For example, there are libraries in which all CDR residues are either Tyr (Y) or Ser(S). Although antibodies (Abs) selected from these libraries show high affinity and specificity, their very unusual composition may make them immunogenic. The present invention is directed toward making Abs that could well have come from the human immune system and so are less likely to be immunogenic. The libraries of the present invention retain as many residues from V-D-J or V-J fusions as possible.

### SUMMARY

Provided are libraries of vectors or packages that encode members of a diverse family of human antibodies comprising heavy chain (HC) CDR3s that are between about 3 amino acids in length to about 35 amino acids in length. The HC CDR3s may also, in certain embodiments, may be rich in Tyr (Y) and Ser(S) and/or comprise diversified D regions and/or comprise extended JH regions. For example, the HC CDR3s may contain greater than about 40% (e.g., between about 43% and about 80%; e.g., greater than about 40% but less than about 100%) Y and/or S residues, e.g., as provided in the examples herein. Also provided are focused libraries comprising such HC CDR3s.

A diversified D region is a D region into which one or more amino acid changes have been introduced (e.g., as compared to the sequence of a naturally occurring D region; for example, a stop codon can be changed to a Tyr residue).

An extended JH region is a JH region that has one or more amino acid residues present at the amino terminus of the framework sequence of the JH region (e.g., amino terminal to FR4 sequences, e.g., which commence with WGQ . . . ). For example, JH1 is an extended JH region. As other examples, JH2, JH3, JH4, JH5, and JH6 are extended JH regions.

Provided also are methods of making and screening the above libraries and the HC CDR3s and antibodies obtained in such screening. Compositions and kits for the practice of these methods are also described herein.

In some aspects, the disclosure features a focused library of vectors or genetic packages that display, display and express, or comprise a member of a diverse family of human antibody related peptides, polypeptides and proteins (e.g., a diverse family of antibodies) and collectively display, display and express, or comprise at least a portion of the diversity of the family, wherein the vectors or genetic packages comprise variegated DNA sequences that encode a heavy chain (HC) CDR3 selected from the group consisting of:
(a) a HC CDR3 that is about 3 or about 4 or about 5 amino acids in length;
(b) a HC CDR3 that is about 23, about 24, about 25, about 26, about 27, about 28, about 29, about 30, about 31, about 32, about 33, about 34 or about 35 amino acids in length (e.g., about 23 to about 35 amino acids in length); and
c) a HC CDR3 that is from about 6 to about 20 amino acids in length (e.g., about 6, about 7, about 8, about 9, about 10, about 11, about 12, about 13, about 14, about 15, about 16, about 17, about 18, about 19, or about 20 amino acids in length);
wherein the HC CDR3 comprises amino acids from a D region (e.g., a diversified D region) (or fragment thereof (e.g., 3 or more amino acids of the D region, e.g., diversified D region)) or a JH region (e.g., an extended JH region).

In some embodiments, the HC CDR3 is enriched in Tyr (Y) and Ser(S) (e.g., greater than 40% of the residues of the HC CDR3 are Y and/or S).

In some embodiments, the library (e.g., the vectors or genetic packages thereof) comprises a D region or a fragment of a D region (e.g., wherein the D region is adjacent to a JH region).

In some embodiments, the library comprises a JH region, e.g., an extended JH region.

In some embodiments, the HC CDR3 comprises amino acids from a D region or a fragment of a D region (e.g., wherein the D region is adjacent to a JH region).

In some embodiments, the D region is selected from the group consisting of D2-2 (RF 2), D2-8 (RF 2), D2-15 (RF 2), D2-21 (RF 2), D3-16 (RF 2), D3-22 (RF 2), D3-3 (RF-2), D3-9 (RF 2), D3-10 (RF 2), D1-26 (RF 3), D4-11 (RF 2), D4-4 (RF 2), D5-5 (RF 3), D5-12 (RF 3), D5-18 (RF 3), D6-6 (RF1), D6-13 (RF 1), and D6-19 (RF 1).

In some embodiments, the HC CDR3 comprises amino acids from a JH region. The JH region may be an extended JH region. In some embodiments, the extended JH region is selected from the group consisting of JH1, JH2, JH3, JH4, JH5, and JH6. In some embodiments, the JH region may be enriched in Y and/or S residues, for example, it may contain greater than about 40% (e.g., between about 43% and about 80%; e.g., greater than about 40% but less than about 100%) Y and/or S residues.

In some embodiments, the D region comprises one or more cysteine (Cys) residues and in some embodiments, the one or more Cys residues are held constant (e.g., are not varied).

In some embodiments, the HC CDR3 (e.g., the DNA encoding the HC CDR3) comprises one or more filling codons between FR3 and the D region and each filling codon is individually NNK, TMY, TMT, or TMC (TMY, TMT, or TMC encode S or Y).

In some embodiments, the HC CDR3 (e.g., the DNA encoding the HC CDR3) comprises one or more filling codons between the D region and JH and each filling codon is individually NNK, TMY, TMT, or TMC.

In some embodiments, the library (e.g., the vectors or genetic packages of the library) further comprises a HC CDR1, HC CDR2, and/or a light chain and also comprises diversity in the HC CDR1, HC CDR2, or light chain comprises diversity in HC CDR1 and/or HC CDR2, and/or a light chain (e.g., kappa or lambda light chain) (respectively). For example, HC CDR3 diversity can be constructed in the background of diversity in HC CDR1, HC CDR2, and/or light chains. For example, the light-chain diversity may be encoded in the same DNA molecule as the HC diversity or the LC and HC diversities may be encoded in separate DNA molecules.

In some aspects, the disclosure features a library comprising a HC CDR3 that is 3, 4, or 5 amino acids in length, wherein the CDR3 comprises amino acids from a JH region (e.g., extended JH region) or from a D region (e.g., a diversified D region) (or fragment thereof (e.g., 3 or more amino acids of the D region, e.g., diversified D region)) joined to the FR4 portion of a JH region.

In some embodiments, the HC CDR3 is from a D region joined to the FR4 portion of a JH region and comprises a trimer, a tetramer, or a pentamer, wherein the trimer, tetramer, or pentamer does not comprise a cysteine residue.

In some embodiments, the HC CDR3 is from a D region joined to the FR4 portion of a JH region and comprises a trimer, a tetramer, or a pentamer, wherein the trimer, tetramer, or pentamer does not comprise a stop codon.

In some embodiments, the D region (e.g., the DNA encoding the D region) comprises a TAG codon and the TAG codon is replaced by a codon selected from the group consisting of TCG, TTG, TGG, CAG, AAG, TAT, and GAG.

In some embodiments, the D region (e.g., the DNA encoding the D region) comprises a TAA codon and the TAA codon is replaced by a codon selected from the group consisting of TCA, TTA, CAA, AAA, TAT, and GAA.

In some embodiments, the D region (e.g., the DNA encoding the D region) comprises a TGA codon and the TGA codon is replaced by a codon selected from the group consisting of TGG, TCA, TTA, AGA, and GGA.

In some embodiments, the library further comprises diversity in HC CDR1 and/or HC CDR2, and/or a light chain (e.g., kappa or lambda light chain). For example, HC CDR3 diversity can be constructed in the background of diversity in HC CDR1, HC CDR2, and/or light chains. For example, the light-chain diversity may be encoded in the same DNA molecule as the HC diversity or the LC and HC diversities may be encoded in separate DNA molecules.

In some aspects, the disclosure provides a method of diversifying a library, the method comprising mutagenizing a library described herein.

In some embodiments, the mutagenizing comprises error-prone PCR.

In some embodiments, the mutagenizing comprises wobbling.

In some embodiments, the mutagenizing comprises dobbling.

In some embodiments, the mutagenizing introduces on average about 1 to about 10 mutations (e.g., about 1, about 2, about 3, about 4, about 5, about 6, about 7, about 8, about 9, about 10 mutations; e.g., base changes) per HC CDR3.

These embodiments of the present invention, other embodiments, and their features and characteristics will be apparent from the description, drawings, and claims that follow.

### DETAILED DESCRIPTION

Antibodies (“Ab”) concentrate their diversity into those regions that are involved in determining affinity and specificity of the Ab for particular targets. These regions may be diverse in sequence or in length. Generally, they are diverse in both ways. However, within families of human antibodies the diversities, both in sequence and in length, are not truly random. Rather, some amino acid residues are preferred at certain positions of the CDRs and some CDR lengths are preferred. These preferred diversities account for the natural diversity of the antibody family.

According to this invention, and as more fully described below, libraries of vectors and genetic packages that encode members of a diverse family of human antibodies comprising heavy chain (HC) CDR3s that are between about 3 to about 35 amino acids in length may be prepared and used. The HC CDR3s may also, in certain embodiments, may be rich in Y and S and/or comprise diversified D regions. Also provided are focused libraries comprising such HC CDR3s.

### Definitions

For convenience, before further description of the present invention, certain terms employed in the specification, examples and appended claims are defined here.

The singular forms “a”, “an”, and “the” include plural references unless the context clearly dictates otherwise.

The term “affinity” or “binding affinity” refers to the apparent association constant or Kₐ. The Kₐ is the reciprocal of the dissociation constant (Kₐ). A binding protein may, for example, have a binding affinity of at least 10⁵, 10⁶, 10⁷, 10⁸, 10⁹, 10¹⁰ and 10¹¹ M⁻¹ for a particular target molecule. Higher affinity binding of a binding protein to a first target relative to a second target can be indicated by a higher K_{A} (or a smaller numerical value K_{D}) for binding the first target than the K_{A} (or numerical value K_{D}) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂) at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

$$[ Bound ] = N \cdot [ Free ] / ( ( 1 / {K}_{A} ) + [ Free ] ) .$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. Heavy chain and light chain may also be abbreviated as HC and LC, respectively. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26 (3): 629-39.)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types, kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH— terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

Herein, the terms “D segment” and “D region” are used interchangeably and are identical. It is to be understood that these items have both DNA and amino-acid representations and that which is meant is clear from the context.

A “library” or “display library” refers to a collection of nucleotide, e.g., DNA, sequences within clones; or a genetically diverse collection of polypeptides displayed on replicable display packages capable of selection or screening to provide an individual polypeptide or a mixed population of polypeptides.

The term “package” as used herein refers to a replicable genetic display package in which the particle is displaying a polypeptide at its surface. The package may be a bacteriophage which displays an antigen binding domain at its surface. This type of package has been called a phage antibody (pAb).

A “pre-determined target” refers to a target molecule whose identity is known prior to using it in any of the disclosed methods.

The term “replicable display package” as used herein refers to a biological particle which has genetic information providing the particle with the ability to replicate. The particle can display on its surface at least part of a polypeptide. The polypeptide can be encoded by genetic information native to the particle and/or artificially placed into the particle or an ancestor of it.

The displayed polypeptide may be any member of a specific binding pair e.g., heavy or light chain domains based on an immunoglobulin molecule, an enzyme or a receptor etc. The particle may be, for example, a virus e.g., a bacteriophage such as fd or M13.

The term “vector” refers to a DNA molecule, capable of replication in a host organism, into which a gene is inserted to construct a recombinant DNA molecule. A “phage vector” is a vector derived by modification of a phage genome, containing an origin of replication for a bacteriophage, but not one for a plasmid. A “phagemid vector” is a vector derived by modification of a plasmid genome, containing an origin of replication for a bacteriophage as well as the plasmid origin of replication.

In discussing oligonucleotides, the notation “[RC]” indicates that the Reverse Complement of the oligonucleotide shown is the one to be used.

### Human Antibody Heavy Chain CDR3s

The heavy chain (“HC”) Germ-Line Gene (GLG) 3-23 (also known as VP-47) accounts for about 12% of all human Abs and is preferred as the framework in the preferred embodiment of the invention. It should, however, be understood that other well-known frameworks, such as 4-34, 3-30, 3-30.3 and 4-30.1, may also be used without departing from the principles of the focused diversities of this invention.

In addition, JH4 (YFDYWGQGTLVTVSS (SEQ ID NO:1)) occurs more often than JH3 in native antibodies. Hence, it is preferred for the focused libraries of this invention. However,

JH3 (AFDIWGQGTMVTVSS (SEQ ID NO: 2)),
JH6 (YYYYYGMDVWGQGTTVTVSS (SEQ ID NO: 3)),

JH1, JH2, or JH5 could as well be used. If present, the double underscored portions of the JHs are considered to be part of CDR3. In Table 3, the FR4 parts of the JHs are underscored.

Naturally, HC CDR3s vary in length. About half of human HCs consist of the components: V::nz::D::ny::JHn where V is a V gene, nz is a series of bases that are essentially random, D is a D segment, often with heavy editing at both ends, ny is a series of bases that are essentially random, and JHn is one of the six JH segments, often with heavy editing at the 5′ end. The D segments appear to provide spacer segments that allow folding of the IgG. The greatest diversity is at the junctions of V with D and of D with JH.

Human D segments have some very strong biases. The tally of the 522 amino-acids in human D segments is Y 70 (13.4%), L 63 (12.1%), V 52 (10%), G 49 (9.4%), I 41 (7.9%), T 40 (7.7%), S 33 (6.3%), W 27 (5.2%), D 21 (4%), A 19 (3.6%), R 16 (3.1%), TAG 15 (2.9%), N 14 2.7%), Q 11 (2.1%), C 9 (1.7%), E 9 (1.7%), F 8 (1.5%), M 8 (1.5%), TGA 8 (1.5%), TAA 7 (1.3%), P 1 (0.2%), H 1 (0.2%), and K 0 (0%). There is one D (2-8 RF 1) that has an unpaired Cys but also a TGA stop codon, so it is little used. Thus, D segments are primarily hydrophobic.

In the preferred libraries of this invention, both types of HC CDR3s are used. In HC CDR3s that have no identifiable D segment, the structure is V::nz::JHn (n=1,6) where JH is usually edited at the 5 end. In HC CDR3s that have an identifiable D segment, the structure is V::nz::D::ny::JHn.

Provided herein are HC CDR3s that are between about 3 to a about 35 amino acids in length. The HC CDR3s may also, in certain embodiments, be rich in Y and S and/or comprise diversified D regions, where a D region is present. For example, the HC CDR3s may contain between about 43% and about 80% Y and/or S residues, e.g., about 43%, about 48%, about 69%, about 63%, about 71%, about 62%, about 58%, about 68%, about 80%, about 77%, or greater than about 40%, or about 40% to less than about 100%, of the residues are Y and/or S. For example, not all of the residues in the CDR3 are Y and/or S. The HC CDR3s may, in certain embodiments, comprise an extended JH region. Exemplary HC CDR3 component designs of the preferred libraries of this invention are shown and described in Examples 1, 2, and 3.

In some embodiments, diversity (e.g., in a CDR, e.g., HC CDR3, or framework region (e.g., framework region near or adjacent to a CDR, e.g., CDR3, e.g., HC CDR3) is generated to create on average about 1, about 2, about 3, about 4, about 5, about 6, about 7, about 8, about 9, about 10, or about 1 to about 10 mutations (e.g., base change), e.g., per CDR (e.g., HC CDR3) or framework region (e.g., framework region near or adjacent to a CDR, e.g., CDR3, e.g., HC CDR3). In some implementations, the mutagenesis is targeted to regions known or likely to be at the binding interface. Further, mutagenesis can be directed to framework regions near or adjacent to the CDRs. In the case of antibodies, mutagenesis can also be limited to one or a few of the CDRs, e.g., to make precise step-wise improvements. Likewise, if the identified ligands are enzymes, mutagenesis can provide antibodies that are able to bind to the active site and vicinity. The CDR or framework region (e.g., an HC CDR3 described herein) may be, in certain embodiments, subjected to error-prone PCR to generate the diversity. This approach uses a “sloppy” version of PCR, in which the polymerase has a fairly high error rate (up to 2%), to amplify the wild-type sequence, and is generally described in Pritchard, et al. (2005) J. Theor. Biol. 234:497-509 and Leung et al. (1989) Technique 1:11-15. Other exemplary mutagenesis techniques include DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zoller et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J. 13:3245).

In some embodiments of the invention, D segments in which a majority of the residues are either Ser or Tyr are picked. In some embodiments, when the DNA encoding the D region is synthesized, each Ser or Tyr residue is encoded by TMT, TMC, or TMY so that the encoded amino acid is either Ser or Tyr.

In some embodiments, the HC CDR3 sequences described herein may be subjected to selection for open reading frames by fusing the sequence encoding the HC CDR3 of interest in frame to an antibiotic resistance gene, such as Kan^{R} gene and selecting for kanamycin resistance. Cells in which the potential CDR3 has a stop codon or a frame shift will not have the antibiotic resistance and that sequence will be eliminated.

### Methods of Construction of Libraries Comprising Human Antibody Heavy Chain CDR3s and Libraries Comprising Human Antibody Heavy Chain CDR3s

An antibody library is a collection of proteins that include proteins that have at least one immunoglobulin variable domain sequence. For example, camelized variable domains (e.g., VH domains) can be used as a scaffold for a library of proteins that include only one immunoglobulin variable domain sequence. In another example, the proteins include two variable domains sequences, e.g., a VH and VL domain, that are able to pair. An antibody library can be prepared from a nucleic acid library (an antibody-coding library) that includes antibody-coding sequences, e.g., comprising the sequences encoding the HC CDR3s provided herein.

In cases where a display library is used, each member of the antibody-coding library can be associated with the antibody that it encodes. In the case of phage display, the antibody protein is physically associated (directly or indirectly) with a phage coat protein. A typical antibody display library member displays a polypeptide that includes a VH domain and a VL domain. The display library member can display the antibody as a Fab fragment (e.g., using two polypeptide chains) or a single chain Fv (e.g., using a single polypeptide chain). Other formats can also be used.

As in the case of the Fab and other formats, the displayed antibody can include one or more constant regions as part of a light and/or heavy chain. In one embodiment, each chain includes one constant region, e.g., as in the case of a Fab. In other embodiments, additional constant regions are included. It is also possible to add one or more constant regions to a molecule after it is identified as having useful antigen binding site. See, e.g., US 2003-0224408.

Antibody libraries can be constructed by a number of processes (see, e.g., de Haard et al. (1999) J. Biol. Chem 274:18218-30; Hoogenboom et al. (1998) Immunotechnology 4:1-20, Hoogenboom et al. (2000) Immunol Today 21:371-8, and Hoet et al. (2005) Nat Biotechnol. 23 (3): 344-8.

In certain embodiments for constructing libraries, the heavy chains comprising the CDR3s described herein and the kappa and lambda light chains are best constructed in separate vectors. First, a synthetic gene is designed to embody each of the synthetic variable domains. The light chains may be bounded by restriction sites for ApaLI (positioned at the very end of the signal sequence) and AscI (positioned after the stop codon). The heavy chain may be bounded by SfiI (positioned within the PelB signal sequence) and NotI (positioned in the linker between CH1 and the anchor protein). Signal sequences other than PelB may also be used, e.g., a M13 pIII signal sequence.

The initial genes may be made with “stuffer” sequences in place of the desired CDRs. A “stuffer” is a sequence that is to be cut away and replaced by diverse DNA, but which does not allow expression of a functional antibody gene. For example, the stuffer may contain several stop codons and restriction sites that will not occur in the correct finished library vector. Stuffers are used to avoid have any one CDR sequence highly represented.

In another embodiment of the present invention, the heavy chain and the kappa or lambda light chains are constructed in a single vector or genetic packages (e.g., for display or display and expression) having appropriate restriction sites that allow cloning of these chains. The processes to construct such vectors are well known and widely used in the art. Preferably, a heavy chain and kappa light chain library and a heavy chain and lambda light chain library would be prepared separately.

Most preferably, the display is on the surface of a derivative of M13 phage. The most preferred vector contains all the genes of M13, an antibiotic resistance gene, and the display cassette. The preferred vector is provided with restriction sites that allow introduction and excision of members of the diverse family of genes, as cassettes. The preferred vector is stable against rearrangement under the growth conditions used to amplify phage.

In another embodiment of this invention, the diversity captured by the methods of the present invention may be displayed and/or expressed in a phagemid vector (e.g., pMID21 (DNA sequence shown in Table 35)) that displays and/or expresses the peptide, polypeptide or protein. Such vectors may also be used to store the diversity for subsequent display and/or expression using other vectors or phage.

In still other embodiments, a method termed the Rapid Optimization of Light Chains or “ROLIC”, described in U.S. Ser. No. 61/028,265 filed Feb. 13, 2008, U.S. Ser. No. 61/043,938 filed Apr. 10, 2008, and U.S. Ser. No. 12/371,000 filed Feb. 13, 2009, a large population of LCs is placed in a phage vector that causes them to be displayed on phage. A small population (e.g., 3, 10, or 25) of HCs are cloned into E. coli so that the HCs are secreted into the periplasm, e.g., those HCs having the CDR3s described herein. The E. coli are then infected with the phage vectors encoding the large population of LCs to produce the HC/LC protein pairings on the phage. The phage particles carry only a LC gene.

In another aspect, in a method termed the Economical Selection of Heavy Chains or “ESCH”, also described in U.S. Ser. No. 61/028,265 filed Feb. 13, 2008, U.S. Ser. No. 61/043,938 filed Apr. 10, 2008, and U.S. Ser. No. 12/371,000 filed Feb. 13, 2009, a small population of LCs may be placed in a vector that causes them to be secreted. A new library of HCs in phage is constructed, such as those provided herein comprising the CDR3s. The LCs and HCs can then be combined by the much more efficient method of infection. Once a small set of effective HC are selected, these can be used as is, fed into ROLIC to obtain an optimal HC/LC pairing, or cloned into a Fab library of LCs for classical selection.

In another embodiment of this invention, the diversity captured by the methods of the present invention may be displayed and/or expressed using a vector suitable for expression in a eukaryotic cell, e.g., a yeast vector, e.g., for expression in a yeast cell.

Other types of protein display include cell-based display (see, e.g., WO 03/029,456); ribosome display (see, e.g., Mattheakis et al. (1994) Proc. Natl. Acad. Sci. USA 91:9022 and Hanes et al. (2000) Nat Biotechnol. 18:1287-92); protein-nucleic acid fusions (see, e.g., U.S. Pat. No. 6,207,446); and immobilization to a non-biological tag (see, e.g., U.S. Pat. No. 5,874,214).

Antibodies isolated from the libraries of the present disclosure may be analyzed to determine the type of the LC and the closest germline gene. In a preferred embodiment, non-germline framework residues are changed back to the germline amino acid so long as binding affinity and specificity are not adversely affected to an unacceptable extent. The substitutions may be done as a group or singly. Human germline sequences are disclosed in Tomlinson, I. A. et al., 1992, J. Mol. Biol. 227:776-798; Cook, G. P. et al., 1995, Immunol. Today 16 (5): 237-242; Chothia, D. et al., 1992, J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). Antibodies are “germlined” by reverting one or more non-germline amino acids in framework regions to corresponding germline amino acids of the antibody, so long as binding properties are substantially retained. Similar methods can also be used in the constant region, e.g., in constant immunoglobulin domains.

For example, an antibody can include one, two, three, or more amino acid substitutions, e.g., in a framework, CDR, or constant region, to make it more similar to a reference germline sequence. One exemplary germlining method can include identifying one or more germline sequences that are similar (e.g., most similar in a particular database) to the sequence of the isolated antibody. Mutations (at the amino acid level) are then made in the isolated antibody, either incrementally or in combination with other mutations. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a framework and/or constant region. For example, a germline framework and/or constant region residue can be from a germline sequence that is similar (e.g., most similar) to the non-variable region being modified. After mutagenesis, activity (e.g., binding or other functional activity) of the antibody can be evaluated to determine if the germline residue or residues are tolerated (i.e., do not abrogate activity). Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

### CDR1, CDR2, and Light-Chain Diversity

It is to be understood that the libraries of HC CDR3 are constructed in the background of diversity in HC CDR1, HC CDR2, and light chains. The light-chain diversity may be encoded in the same DNA molecule as the HC diversity or the LC and HC diversities may be encoded in separate DNA molecules. In Table 22 the fusion of a signal sequence:: VH::CH1::His6::Myc::IIIstump. CDR1 comprises residues 31-35; there is diversity at residues 31, 33, and 35. In one embodiment, residues 31, 33, and 35 can be any amino-acid type except cysteine. CDR2 comprises residues 50 through 65. There is diversity at positions 50, 52, 52a, 56, and 58. In one embodiment, residues 50, and 52 can be any of the types Ser, Gly, Val, Trp, Arg, Tyr; residue 52a can be Pro or Ser and residues 56 and 58 can be any amino-acid type except Cys. The diversity of HC CDR3 is cloned into a diversity of HC CDR1 and 2 that is at least 1. E4, 1. E5, 1. E6, 1.E7, 5. E 7, or 1. E 8.

In one embodiment, residues 31, 33, 35, 50, 52, 56, and 58 can be any amino-acid type except Cys and residue 52a can be Gly, Ser, Pro, or Tyr. The diversity of HC CDR3 is cloned into a diversity of HC CDR1 and 2 that is at least 1. E4, 1. E5, 1. E6, 1. E7, 5. E 7, or 1. E 8.

In one embodiment, the diversity of the HC is cloned into a vector (phage or phagemid) that contains a diversity of light chains. This diversity is at least 25, 50, 100, 500, 1. E 3, 1. E 4, 1. E5, 1. E 6, or 1. E7. The diversity of HC CDR3 is at least 221, 272, 500, 1000, 1. E 4, 1. E 5, 1. E6, 1. E7, or 1. E 8.

In one embodiment, the diversity of the HC is cloned into a phage vector that displays the HC on a phage protein such as III, VIII, VII, VI, or IX or a fragment of one of these sufficient to cause display and light chains are combined with the HC by infecting a cell collection wherein each cell secrets a light chain. The diversity of the light chains in the cells is at least 5, 10, 15, 20, 25, 30, 35, 40, 50, 75, or 100. The diversity of HC CDR3 is at least 221, 272, 500, 1000, 1. E 4, 1. E5, 1. E 6, 1. E7, or 1. E 8.

Table 30 shows the sequence of the phage vector DY3FHC87 (SEQ ID NO:894) which carries a bla gene, a display cassette for heavy chains under control of a P_{lac} promoter. DY3FHC87 contains all the genes of M13 as well. Infecting F+E. coli cells that harbor a diversity of light chains in a vector such as pLCSK23 (Sequence in Table 40) (SEQ ID NO:896). The vector pLCSK23 carries a Kan^{R} gene. Under the control of P_{lac} promoter, there is a gene beginning at base 2215 having a signal sequence (bases 2215-2277), a VL (in this sequence the VL encodes the sequence shown in (SEQ ID NO:897) from base 2278 to base 2598, Ckappa from base 2599 to 2922, a linker that allows an NotI site from 2923 to 2931, and a V5 tag (bases 2932-2973). There are an SfiI site at 2259-2271 and a KpnI site at 2602-2605 to allow easy replacement of Vkappas. (SEQ ID NO:897) is an example of the proteins that are secreted. It is to be understood that CKappa and the V5 tag are constant. All of the proteins shown in Table 19 (VK102gl-JK3, VK102 var1, VK102 var2, VK1O2 var3, VK102 var4, VK102 var5, VK3L6gl-JK4, VK3L6 var1, VK3L6 var2, VK3L6 var3, VK3L6 var4, VK3L6 var5, VK3L6 var6, VK3L6 var7, VK3L6 var8, VK3A27gl-JK3, VK3A27 var1, VK3A27 var2, VK3A27 var3, VK3A27 var4, VK3A27 var5, VK3A27 var6, VK3A27 var7, VK3L2gl-JK3, and VK1gIL8-JK5) will have these sequences attached at the carboxy end.

### Methods of Using the Libraries

Off-Rate Selection. Since a slow dissociation rate can be predictive of high affinity, particularly with respect to interactions between polypeptides and their targets, the methods described herein can be used to isolate ligands with a desired kinetic dissociation rate (i.e., reduced) for a binding interaction to a target.

To select for slow dissociating antibodies from a display library, the library is contacted to an immobilized target. The immobilized target is then washed with a first solution that removes non-specifically or weakly bound antibodies. Then the bound antibodies are eluted with a second solution that includes a saturating amount of free target, i.e., replicates of the target that are not attached to the particle. The free target binds to antibodies that dissociate from the target. Rebinding of the eluted antibodies is effectively prevented by the saturating amount of free target relative to the much lower concentration of immobilized target.

The second solution can have solution conditions that are substantially physiological or that are stringent (e.g., low pH, high pH, or high salt). Typically, the solution conditions of the second solution are identical to the solution conditions of the first solution. Fractions of the second solution are collected in temporal order to distinguish early from late fractions. Later fractions include antibodies that dissociate at a slower rate from the target than biomolecules in the early fractions. Further, it is also possible to recover antibodies that remain bound to the target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards antibodies that bind to a non-target molecule. Examples of non-target molecules include, e.g., a carbohydrate molecule that differs structurally from the target molecule, e.g., a carbohydrate molecule that has a different biological property from the target molecule. In the case of a sulfated carbohydrate, a non-target may be the same carbohydrate without the sulfate or with the sulfate in a different position. In the case of a phosphopeptide, the non-target may be the same peptide without the phosphate or a different phosphopeptide.

In one implementation, a so-called “negative selection” step is used to discriminate between the target and related non-target molecule and a related, but distinct non-target molecules. The display library or a pool thereof is contacted to the non-target molecule. Members that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target.

In certain embodiments, the antibodies comprising the CDR3s of the invention may be able to bind carbohydrates. Methods for evaluating antibodies for carbohydrate binding include ELISA, immunohistochemistry, immunoblotting, and fluorescence-activated cell sorting. These methods can be used to identify antibodies which have a K_{D} of better than a threshold, e.g., better than 100 nM, 50 nM, 10 nM, 5 nM, 1 nM, 500 PM, 100 pM, or 10 pM.

ELISA. Proteins encoded by a display library can also be screened for a binding property using an ELISA assay. For example, each protein is contacted to a microtitre plate whose bottom surface has been coated with the target, e.g., a limiting amount of the target. The plate is washed with buffer to remove non-specifically bound polypeptides. Then the amount of the protein bound to the plate is determined by probing the plate with an antibody that can recognize the polypeptide, e.g., a tag or constant portion of the polypeptide. The antibody is linked to an enzyme such as alkaline phosphatase, which produces a calorimetric product when appropriate substrates are provided. The protein can be purified from cells or assayed in a display library format, e.g., as a fusion to a filamentous bacteriophage coat. Alternatively, cells (e.g., live or fixed) that express the target molecule, e.g., a target that contains a carbohydrate moiety, can be plated in a microtitre plate and used to test the affinity of the peptides/antibodies present in the display library or obtained by selection from the display library.

In another version of the ELISA assay, each polypeptide of a diversity strand library is used to coat a different well of a microtitre plate. The ELISA then proceeds using a constant target molecule to query each well.

Cell Binding Assays. Antibodies can be evaluated for their ability to interact with one or more cell types, e.g., a hematopoietic cell. Fluorescent activated cell sorting (FACS) is one exemplary method for testing an interaction between a protein and a cell. The antibody is labeled directly or indirectly with a fluorophore, before or after, binding to the cells, and then cells are counted in a FACS sorter.

Other cell types can be prepared for FACS by methods known in the art.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{D}), and kinetic parameters, including kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow k_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow k_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Proteins identified from the display library can be immobilized on a solid support, for example, on a bead or an array. For a protein array, each of the polypeptides is immobilized at a unique address on a support. Typically, the address is a two-dimensional address. Methods of producing polypeptide arrays are described, e.g., in De Wildt et al. (2000) Nat. Biotechnol. 18:989-994; Lucking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

### Kits

Also provided are kits for use in carrying out a method according to any aspect of the invention. The kits may include the necessary vectors. One such vector will typically have an origin of replication for single stranded bacteriophage and either contain the sbp member nucleic acid or have a restriction site for its insertion in the 5′ end region of the mature coding sequence of a phage capsid protein, and with a secretory leader coding sequence upstream of said site which directs a fusion of the capsid protein exogenous polypeptide to the periplasmic space.

Also provided are packages encoding the HC CDR3s as defined above and polypeptides comprising the HC CDR3s and fragments and derivatives thereof, obtainable by use of any of the above defined methods. The derivatives may comprise polypeptides fused to another molecule such as an enzyme or a Fc tail.

The kit may include a phage vector (e.g., DY3F87HC) which has a site for insertion of HC CDR3s for expression of the encoded polypeptide in free form. The kit may also include a plasmid vector for expression of soluble light chains, e.g., pLCSK23. The kit may also include a suitable cell line (e.g., TG1). The diversity of light chains encoded by pLCSK23 may be 10, 15, 20, 25, 30, or 50. The LCs in the diversity may be constructed or picked to have certain desirable properties, such as, being germline in the framework regions and having diversity in CDR3 and/or CDR1. The germlines may be of highly utilized ones, e.g., VK1_2-02, VK3_1-A27, VK3_5-L6, VK3_3-L2 for kappa and VL2_2a2, VL1_1c, VL1_1g, VL3_3r for lambda.

For example, one could clone genes for

VK102gl-JK3, VK102 var1, VK102 var2, VK1O2 var3, VK1O2 var4, VK102 var5, VK3L6gl-JK4, VK3L6 var1, VK3L6 var2, VK3L6 var3, VK3L6 var4, VK3L6 var5, VK3L6 var6, VK3L6 var7, VK3L6 var8, VK3A27gl-JK3, VK3A27 var1, VK3A27 var2, VK3A27 var3, VK3A27 var4, VK3A27 var5, VK3A27 var6, VK3A27 var7, VK3L2gl-JK3, VK1gIL8-JK5, and VK1GLO12-JK3 (amino-acid sequences shown in Table 19) into pLCSK23.

TABLE 19
26 VL to be used in pLCSK23.

VK1O2gl-JK3
(SEQ ID NO: 4)

| | |
|---|---|
| DIQMTQSPSS LSASVGDRVT ITCRASQSIS SYLNWYQQKP GKAPKLLIYA ASSLQSGVPS | 60 |
| RFSGSGSGTD FTLTISSLQP EDFATYYCQQ SYSTPFTFGP GTKVDIK | 107 |
| VK1O2var1 | |

(SEQ ID NO: 5)

| | |
|---|---|
| S28D | |
| DIQMTQSPSS LSASVGDRVT ITCRASQDIS SYLNWYQQKP GKAPKLLIYA ASSLQSGVPS | 60 |
| RFSGSGSGTD FTLTISSLQP EDFATYYCQQ SYSTPFTFGP GTKVDIK | 107 |
| VK1O2var2 | |

(SEQ ID NO: 6)

| | |
|---|---|
| S91R | |
| DIQMTQSPSS LSASVGDRVT ITCRASQSIS SYLNWYQQKP GKAPKLLIYA ASSLQSGVPS | 60 |
| RFSGSGSGTD FTLTISSLQP EDFATYYCQQ RYSTPFTFGP GTKVDIK | 107 |
| VK1O2var3 | |

(SEQ ID NO: 7)

| | |
|---|---|
| S91E | |
| DIQMTQSPSS LSASVGDRVT ITCRASQSIS SYLNWYQQKP GKAPKLLIYA ASSLQSGVPS | 60 |
| RFSGSGSGTD FTLTISSLQP EDFATYYCQQ EYSTPFTFGP GTKVDIK | 107 |
| VK1O2var4 | |

(SEQ ID NO: 8)

| | |
|---|---|
| S31R | |
| DIQMTQSPSS LSASVGDRVT ITCRASQSIS RYLNWYQQKP GKAPKLLIYA ASSLQSGVPS | 60 |
| RFSGSGSGTD FTLTISSLQP EDFATYYCQQ SYSTPFTFGP GTKVDIK | 107 |
| VK1O2var5 | |

(SEQ ID NO: 9)

| | |
|---|---|
| S31E, S93R | |
| DIQMTQSPSS LSASVGDRVT ITCRASQSIS EYLNWYQQKP GKAPKLLIYA ASSLQSGVPS | 60 |
| RFSGSGSGTD FTLTISSLQP EDFATYYCQQ SYRTPFTFGP GTKVDIK | 107 |
| VK3L6gl-JK4 | |

(SEQ ID NO: 10)

| | |
|---|---|
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RSNWPLTFGG GTKVEIK | 107 |
| VK3L6var1 | |

(SEQ ID NO: 11)

| | |
|---|---|
| S31R | |
| EIVLTQSPAT LSLSPGERAT LSCRASQSVS RYLAWYQQKP GQAPRLLIYD ASNRATGIPA | 60 |
| RFSGSGSGTD FTLTISSLEP EDFAVYYCQQ RSNWPLTFGG GTKVEIK | 107 |
| VK3L6var2 | |

(SEQ ID NO: 12)

| | |
|---|---|
| S92R | |
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RRNWPLTFGG GTKVEIK | 107 |
| VK3L6var3 | |

(SEQ ID NO: 13)

| | |
|---|---|
| S92G | |
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RGNWPLTFGG GTKVEIK | 107 |
| VK3L6var4 | |

(SEQ ID NO: 14)

| | |
|---|---|
| S92Y | |
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RYNWPLTFGG GTKVEIK | 107 |
| VK3L6var5 | |

(SEQ ID NO: 15)

| | |
|---|---|
| S92E | |
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RENWPLTFGG GTKVEIK | 107 |
| VK3L6var6 | |

(SEQ ID NO: 16)

| | |
|---|---|
| Y32F | |
| EIVLTQSPAT LSLSPGERAT LSCRASQSVS SFLAWYQQKP GQAPRLLIYD ASNRATGIPA | 60 |
| RFSGSGSGTD FTLTISSLEP EDFAVYYCQQ RSNWPLTFGG GTKVEIK | 107 |
| VK3L6var7 | |

(SEQ ID NO: 17)

| | |
|---|---|
| Y32D | |
| EIVLTQSPAT LSLSPGERAT LSCRASQSVS SDLAWYQQKP GQAPRLLIYD ASNRATGIPA | 60 |
| RFSGSGSGTD FTLTISSLEP EDFAVYYCQQ RSNWPLTFGG GTKVEIK | 107 |
| VK3L6var8 | |

(SEQ ID NO: 18)

| | |
|---|---|
| N93G | |
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ RSGWPLTFGG GTKVEIK | 107 |
| VK3A27gl-JK3 | |

(SEQ ID NO: 19)

| | |
|---|---|
| EIVLTQSPGT LSLSPGERAT LSCRASQSVS SSYLAWYQQK PGQAPRLLIY GASSRATGIP | 60 |
| DRFSGSGSGT DFTLTISRLE PEDFAVYYCQ QYGSSPFTFG PGTKVDIK | 108 |
| VK3A27var1 | |

(SEQ ID NO: 20)

| | |
|---|---|
| S31R | |
| EIVLTQSPGT LSLSPGERAT LSCRASQSVS RSYLAWYQQK PGQAPRLLIY GASSRATGIP | 60 |
| DRFSGSGSGT DFTLTISRLE PEDFAVYYCQ QYGSSPFTFG PGTKVDIK | 108 |
| VK3A27var2 | |

(SEQ ID NO: 21)

| | |
|---|---|
| S32R | |
| EIVLTQSPGT LSLSPGERAT LSCRASQSVS SRYLAWYQQK PGQAPRLLIY GASSRATGIP | 60 |
| DRFSGSGSGT DFTLTISRLE PEDFAVYYCQ QYGSSPFTFG PGTKVDIK | 108 |
| VK3A27var3 | |

(SEQ ID NO: 22)

| | |
|---|---|
| S32D | |
| EIVLTQSPGT LSLSPGERAT LSCRASQSVS SDYLAWYQQK PGQAPRLLIY GASSRATGIP | 60 |
| DRFSGSGSGT DFTLTISRLE PEDFAVYYCQ QYGSSPFTFG PGTKVDIK | 108 |
| VK3A27var4 | |

(SEQ ID NO: 23)

| | |
|---|---|
| G93E | |
| EIVLTQSPGT LSLSPGERAT LSCRASQSVS SSYLAWYQQK PGQAPRLLIY GASSRATGIP | 60 |
| DRFSGSGSGT DFTLTISRLE PEDFAVYYCQ QYESSPFTFG PGTKVDIK | 108 |
| VK3A27var5 | |

(SEQ ID NO: 24)

| | |
|---|---|
| G93R | |
| EIVLTQSPGT LSLSPGERAT LSCRASQSVS SSYLAWYQQK PGQAPRLLIY GASSRATGIP | 60 |
| DRFSGSGSGT DFTLTISRLE PEDFAVYYCQ QYRSSPFTFG PGTKVDIK | 108 |
| VK3A27var6 | |

(SEQ ID NO: 25)

| | |
|---|---|
| S30D, G93E | |
| EIVLTQSPGT LSLSPGERAT LSCRASQSVD SSYLAWYQQK PGQAPRLLIY GASSRATGIP | 60 |
| DRFSGSGSGT DFTLTISRLE PEDFAVYYCQ QYESSPFTFG PGTKVDIK | 108 |
| VK3A27var7 | |

(SEQ ID NO: 26)

| | |
|---|---|
| S94R | |
| EIVLTQSPGT LSLSPGERAT LSCRASQSVS SSYLAWYQQK PGQAPRLLIY GASSRATGIP | 60 |
| DRFSGSGSGT DFTLTISRLE PEDFAVYYCQ QYGRSPFTFG PGTKVDIK | 108 |
| VK3L2gl-JK3 | |

(SEQ ID NO: 27)

| | |
|---|---|
| EIVMTQSPAT LSVSPGERAT LSCRASQSVS SNLAWYQQKP GQAPRLLIYG ASTRATGIPA | 60 |
| RFSGSGSGTE FTLTISSLQS EDFAVYYCQQ YNNWPFTFGP GTKVDIK | 107 |
| VK1glL8-JK5 | |

(SEQ ID NO: 28)

| | |
|---|---|
| DIQLTQSPSF LSASVGDRVT ITCRASQGIS SYLAWYQQKP GKAPKLLIYA ASTLQSGVPS | 60 |
| RFSGSGSGTE FTLTISSLQP EDFATYYCQQ LNSYPITFGQ GTRLEIK | 107 |
| VK1GLO12-JK3 | |

(SEQ ID NO: 897)

| | |
|---|---|
| DIQMTQSPSS LSASVGDRV TITCRASQSI SSYLNWYQQK PGKAPKLLIY AASSLQSGVP | 60 |
| SRFSGSGSGT DFTLTISSL QPEDFATYYC QQSYSTPFTF GPGTKVDIKR GTVAAPSVFI | 120 |
| FPPSDEQLKS GTASVVCLL NNFYPREAKV QWKVDNALQS GNSQESVTEQ DSKDSTYSLS | 180 |
| STLTLSKADY EKHKVYACE VTHQGLSSPV TKSFNRGECA AAGKPIPNPL LGLDST | 236 |

The kits may include ancillary components required for carrying out the method, the nature of such components depending of course on the particular method employed. Useful ancillary components may comprise helper phage, PCR primers, buffers, and/or enzymes of various kinds. Buffers and enzymes are typically used to enable preparation of nucleotide sequences encoding Fv, scFv or Fab fragments derived from rearranged or unrearranged immunoglobulin genes according to the strategies described herein.

### Methods of Introducing Diversity

There are many ways of generating DNA that is variable. One way is to use mixed-nucleotide synthesis (MNS). One version of MNS uses equimolar mixtures of nucleotides as shown in Table 5. For example, using NNK codons gives all twenty amino acids and one TAG stop codon. The distribution is 3 (R/S/L): 2 (A/G/V/T/P): 1 (C/D/E/F/H/I/K/M/N/Q/W/Y) (e.g., 3 of each of Arg, Ser, and Leu, and so forth). An alternative, herein termed “wobbling”, uses mixed nucleotides but not in equimolar amounts.

For example, if a parental codon were TTC (encoding Phe), we could use a mixture of (0.082 T, 0.06 C, 0.06 A, and 0.06 G) in place of T and a mixture of (0.082 C, 0.06 T, 0.06 A, and 0.06 G) in place of C. This would give TTC or TTT (encoding Phe) 59% of the time and Leu 13%, S/V/I/C/Y ˜5%, and other amino-acid types less often.

Van den Brulle et al. (Biotechniques 45:340-3 (2008)) describe a method of synthesis of variable DNA in which type IIs restriction enzymes are used to transfer trinucleotides from an anchored hair-pin oligonucleotide (PHONs) to a so called “splinker”. By using mixtures of anchored PHONs and splinkers, one can build libraries in which desired amino-acid types are allowed in designer-determined ratios. Thus, one can direct that one amino-acid type is present, for example 82% of the time and 18 other amino-acid types (all non-parental amino-acid types except Cys) are present at 2% each. Herein, we will refer to such a synthesis as “dobbling” (digital wobbling). In some aspects, dobbling is preferred to wobbling, but wobbling provides useful embodiments, partly because the structure of the genetic code table causes wobbling to make mostly conservative substitutions. Dobbling does offer the possibility to exclude unwanted amino-acid types. In CDRs, unpaired cysteines are known, even in Abs approved as therapeutics, but in some embodiments, one would like to avoid them. In some embodiments, when diversifying a D region that contains a pair of cysteines, the cysteins are not allowed to vary because the disulfide-closed loop is an important structural element and because one does not want unpaired cysteines.

In addition, one can synthesize a DNA molecule that encodes a parental amino-acid sequence and subject that DNA to error-prone PCR using primers that cover the framework regions so that mutations in the framework regions are avoided.

TABLE 5
Standard codes for mixed nucleotides

| | |
|---|---|
| N is equimolar A, C, G, T | |
| B is equimolar C, G, T | (not A) |
| D is equimolar A, G, T | (not C) |
| H is equimolar A, C, T | (not G) |
| V is equimolar A, C, G | (not T) |
| K is equimolar G, T | (Keto) |
| M is equimolar A, C | (aMino) |
| R is equimolar A, G | (puRine) |
| S is equimolar C, G | (Strong) |
| W is equimolar A, T | (weak) |
| Y is equimolar C, T | (pYrimidine) |

TABLE 6
Example of mixed nucleotides for wobbling
e = 0.82 A + 0.06 C + 0.06 G + 0.06 T
q = 0.06 A + 0.82 C + 0.06 G + 0.06 T
j = 0.06 A + 0.06 C + 0.82 G + 0.06 T
z = 0.06 A + 0.06 C + 0.06 G + 0.82 T

### Exemplification

The present invention is further illustrated by the following examples which should not be construed as limiting in any way. The contents of all references, pending patent applications and published patents, cited throughout this application are hereby expressly incorporated by reference.

### Prophetic Example 1: Libraries With Very Short HC CDR3s

Very short HC CDR3s have been described in the art. Kadirvelraj et al. (2006) Proc. Natl. Acad. Sci. USA 103:8149-54 have described a four amino-acid HC CDR3 sequence in an antibody that binds Streptococcus Type B III Ag (GBS-Ag) but not to Streptococcus pneumoniae capsular Ag. GBS-Ag is sialylated at regular intervals. S. pneumoniae capsular Ag (SPC-Ag) is very similar but lacks the sialic acid groups. Such a short HC CDR3 creates a wide groove into which a carbohydrate could bind, and such Abs are very, very rare in existing antibody libraries. Thus, current libraries do not afford a large variety of potential binders to carbohydrates.

Ab 1B1 is the murine mAb that binds GBS-Ag; Ab 1QFU is the mAb having a known 3D structure and the closest sequence; and INSN is an antibody of known 3D structure having a HC CDR3 of length 4. Examination of a 3-23 HC structure gives a distance from Ca of R94 (which ends FR3) to the Ca of the W 104 (which begins FR4) of ˜10 Å. The CDR3 of 1B1 (NWDY (SEQ ID NO:29)) shows that the AAs need not have only small side groups or be mostly of glycine. Three amino acids (AAs) can bridge 10 Å, although PPP might not work. Indeed, we have obtained a few Fabs with CDR3s as short as 3 AAs, but they are very rare.

Although short and very short HC CDR3s have been described, no one has suggested making an Ab library having many members (e.g., greater than about 50%, about 60%, about 70%, about 80%, about 90%, or about 95% of members) with short HC CDR3s (e.g., HC CDR3s of 3 to 5 amino acids). One approach to building an effective library is to first design amino-acid sequences that could arise from V-J or V-D-J coupling. For CDR3 length 3, 4, or 5, we start with the amino-acid sequences shown in Table 7. For example, Sequence V-3JH1 shows the C-terminal end of 3-23 FR3 (TAVYYCAK (SEQ ID NO:30)) followed by JH1 which has been trimmed from the N-terminal end until three amino-acids before the Trp-Gly that starts FR4. V-3JH2 shows the end of FR3 followed by the trimmed JH2. The sequence following V-3JH6 are constructed by joining FR4 to a trimer taken from a human D segment followed by the FR4 region of a human JH segment. 3D3-3.3.2 would be a trimer from segment D3-3, third reading frame starting at the second amino acid. 5D5-12.2.3 is a pentamer from D5-12 in reading frame 2 starting at amino acid 3. Some of the germ-line D segments contain stop codons, yet they appear in natural antibodies when the stop codons are edited away. Here we assume that the most likely change fro TAA and TAG codons is to Tyr (Y) and that TGA stops are most likely mutated to Trp (W). Table 20 shows the amino-acid sequences of the human D segments; the types of stop codons is indicated by the use of * for TAG, @ for TAA, and $ for TGA. In Table 11 are 266 distinct trimers that can be constructed from human D segments. The TAA and TAG stops have been changed to Tyr shown as “y” (i.e., lowercase). These could also be changed to Ser, Cys, Phe, Gln, Lys, or Glu by single base changes. TAG could be changed by single base changes to Trp as well as Tyr, Gln, Lys, Glu, Ser, and Leu. Table 12 shows the 266 distinct tetramers that can be obtained by trimming human D segments. Table 13 shows the 215 pentamers that can be obtained from trimming human D segments. Table 14 shows the 155 hexamers that can be obtained by trimming human D segments. The libraries to be built have substantial diversity in HC CDR1 and HC CDR2. The sequence diversity of HC CDR3 may be less important than having a short, but acceptable sequence. The diversity of JH segments or fragments (e.g., 3 or more amino acids) of D segments provides sequences that could be built by the human immune system and so are less likely to be immunogenic.

In one embodiment, the trimers, tetramers, and pentamers that contain a Cys are eliminated.

In one embodiment, the trimers, tetramers, and pentamers that contain a Cys or the came from a D fragment containing a stop are eliminated.

The short libraries constructed using the trimers of Table 11, tetramers of Table 12, pentamers of Table 13 have substantial diversity: 266, 266, and 215 respectively. This is to be compared to the number of peptides of these lengths: 8000, 160000, and 3200000 respectively.

V-3D1-1.1.1-JH1 contains the final portion of FR3 followed by three amino acids from D1-1 (RF1), viz. GTT (SEQ ID NO:257). V-3D1-1.2-JH1 uses amino acids 2-4 of D1-1 (RF1) as the parental CDR3. V-3D3-3.3.3-JH2 shows the end of FR3 followed by amino acids 3-5 of D3-3 (RF 3). The invention comprises any amino-acid sequence comprising FR3::(three, four, or five stop-free AAs of a human D segment)::FR4 from a human JH. Fragments of D regions containing unpaired Cys residues are less preferred than those that are free of unpaired Cys residues. In V-5JH3, there is a Tyr shown as ‘y’ because JH3 has only 4 codons before the codons for Trp-Gly that define the beginning of FR4. V-5JH4 has a Ser shown as ‘s’ for the same reason. If wobbling is used, the preferred level of purity is between 0.75 and 0.90. The invention comprises the sequences V-3JH1 through V-3JH6, V-4JH1 through V-4JH6, and V-5JH1 through V-5JH6, and libraries containing the same The invention also comprises the sequences in which the CDR region is replaced by a 3, 4, or 5 amino-acid segment from a human D region, and libraries containing the same. The invention further comprises DNA in which the parental sequence has been mutated in the CDR3 region, and libraries containing the same. A preferred embodiment is one in which the average number of base changes per CDR3 is one, two, or three. The methods of mutagenesis include error-prone PCR, wobbling, and dobbling.

TABLE 7
Amino-acid sequences of parental CDR3s

Length 3

| | ...FR3----- CDR3- FR4-------- | |
|---|---|---|
| V-3JH1 | TAVYYCAK FQH WGQGTLVTVSS | (SEQ ID NO: 31) |
| V-3JH2 | TAVYYCAK FDL WGRGTLVTVSS | (SEQ ID NO: 32) |
| V-3JH3 | TAVYYCAK FDI WGQGTMVTVSS | (SEQ ID NO: 33) |
| V-3JH4 | TAVYYCAK FDY WGQGTLVTVSS | (SEQ ID NO: 34) |
| V-3JH5 | TAVYYCAK FDP WGQGTLVTVSS | (SEQ ID NO: 35) |
| V-3JH6 | TAVYYCAK MDV WGQGTTVTVSS | (SEQ ID NO: 36) |
| V-3D1-1.1.1-JH1 | TAVYYCAK GTT WGQGTLVTVSS | (SEQ ID NO: 37) |
| V-3D1-1.1.2-JH1 | TAVYYCAK TTG WGQGTLVTVSS | (SEQ ID NO: 38) |
| V-3D3-3.3.3-JH2 | TAVYYCAK IFG WGRGTLVTVSS | (SEQ ID NO: 39) |
| Length 4 | | |
| V-4JH1 | TAVYYCAK YFQH WGQGTLVTVSS | (SEQ ID NO: 40) |
| V-4JH2 | TAVYYCAK YFDL WGRGTLVTVSS | (SEQ ID NO: 41) |
| V-4JH3 | TAVYYCAK AFDI WGQGTMVTVSS | (SEQ ID NO: 42) |
| V-4JH4 | TAVYYCAK YFDY WGQGTLVTVSS | (SEQ ID NO: 43) |
| V-4JH5 | TAVYYCAK WFDP WGQGTLVTVSS | (SEQ ID NO: 44) |
| V-4JH6 | TAVYYCAK GMDV WGQGTTVTVSS | (SEQ ID NO: 45) |
| V-4D3-10.1a-JH2 | TAVYYCAK LLWF WGRGTLVTVSS | (SEQ ID NO: 46) |
| Length 5 | | |
| V-5JH1 | TAVYYCAK EYFQH WGQGTLVTVSS | (SEQ ID NO: 47) |
| V-5JH2 | TAVYYCAK WYFDL WGRGTLVTVSS | (SEQ ID NO: 48) |
| V-5JH3 | TAVYYCAK yAFDI WGQGTMVTVSS | (SEQ ID NO: 49) |
| V-5JH4 | TAVYYCAK sYFDY WGQGTLVTVSS | (SEQ ID NO: 50) |
| V-5JH5 | TAVYYCAK NWFDP WGQGTLVTVSS | (SEQ ID NO: 51) |
| V-5JH6 | TAVYYCAK YGMDV WGQGTTVTVSS | (SEQ ID NO: 52) |
| V-5D2-8.2a-JH2 | TAVYYCAK DIVLM WGRGTLVTVSS | (SEQ ID NO: 53) |

TABLE 8
DNA encoding V-5D2-8.2a-JH2 for wobbling

```
! CDR3.......
! A E D T A V Y Y C A K D I V L M
    |gct|gag|gaT|aCT|GCA|GtT|taT|taC|tgc|gct aag jez ezq jzz qzz ezj
!
! W G Q G T T V T V S S (SEQ ID NO: 54)
        tgg ggc cag ggt act acG GTC ACC gtc tcc agt-3′ (SEQ ID NO: 55)
! BstEII...
```

Alternatively, one could synthesize three fragments of DNA that correspond to the region from XbaI to BstEII and having residue 94 being K or R followed by 3, 4, or 5 NNK codons, followed by WG . . . of FR4. The allowed variation is 20³+20⁴+20⁵=3,368,000. After amplification, these DNA molecules would be mixed in the ratio 1:10:100 (so that shorter sequences are relatively oversampled) and cloned into the phagemid encoding the kappa library with HC CDR1/2 diversity. A library of 1×10⁹ would give significant diversity and will allow isolation of antibodies that bind to targets that have small to medium protrusions. For example, various carbohydrates, loops of proteins that are not well ordered (such as GPCRs) may benefit from a groove in the antibody created by having a very short HC CDR3. We can also build a lambda library. The ratio of AA sequences is 1:20:400, and it may be important to sample the shorter sequences more densely. Getting a big, wide gulley in the Ab may require exactly one 3 AA CDR3, but with a 4 AA CDR3, one probably has more leeway and with 5 AAs, even more leeway. In this Example, we use the JH6 version of FR4 from the WG motif onward.

We can select from our current kappa library a collection of, for example, 25 kappa light chains that are a) germline in the framework regions, b) show suitable diversity in CDRs, and c) are of types that produce well and pair well with 3-23. These LCs will be made in E. coli from a vector that carries Kan^{R} and no phage packaging signal. We would then build our HC library in a phage vector that has no LC. HC and LC will be crossed by infecting the LC producing cells with the HC phage. HC phage that are selected can be combined with the LC of the cell that produces ELISA⁺ phage or the HCs can be cloned into pMID21 that have the whole LC diversity. Alternatively, the selected HC can be moved into pHCSK85 and used with ROLIC to combine with all the LCs of our collection. Lambda LCs could also be used. Thus, a library of 1×10⁹ HC in phage can be expanded into a Fab library of 1.2×10¹¹ (1.×10⁹×117). If we combined 1×10⁷ CDR1-2s with 10⁶ HC CDR3s, we could make a library of 5×10⁷ in which each CDR3 is coupled with 50 CDR1-2s. A library of 5×10⁷ HCs in phage could give results similar to an old-style library of 6×10⁹.

TABLE 1
Designs of very short exemplary HC CDR3s

```
C3XXX
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! CDR3.......
! L R A E D T A V Y Y C A K|R any any any W G
    |TTA|AGg|gct|gag|gaT|aCT|GCA|GtT|taT|taC|tgc|gct aRg nnk nnk nnk tgg ggc-
!
! Q G T T V T V S S (SEQ ID NO: 56)
      cag ggt act acG GTC ACC gtc tcc agt-3′ (SEQ ID NO: 57)
! BstEII...
!
(C3XXX)5′-T|GCA|GtT|taT|taC|tgc|gct aRg nnk nnk nnk tgg ggc cag ggt act ac-3′
(SEQ ID NO: 58)
(ON_5) 5′-AcTggAgAcggTgAccgTAgTAcccTggccccA-3′ ! 33 bases SEQ ID NO: 58)
(ON_5 is reverse complement of 5′-tgg ggc cag ggt act acG GTC ACC gtc tcc
agt-3′) (SEQ ID NO: 59)
! Use ON-1 and ON-3 shown below
!-----------------------------------------------
!
C3X4
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! CDR3...........
! L R A E D T A V Y Y C A K|R any any any any W
    |TTA|AGg|gct|gag|gaT|aCT|GCA|GtT|taT|taC|tgc|gct aRg nnk nnk nnk nnk tgg-
!
! G Q G T T V T V S S (SEQ ID NO: 60)
      ggc cag ggt act acG GTC ACC gtc tcc agt-3′ (SEQ ID NO: 61)
! BstEII...
!
(C3X4)5′-GCA|GtT|taT|taC|tgc|gct aRg nnk nnk nnk nnk tgg-
                      ggc cag ggt act ac-3′ (SEQ ID NO: 62)
! Use ON-1, ON-3, and ON-5
!----------------------------------------------------------
C3X5
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! CDR3...............
! L R A E D T A V Y Y C A K|R any any any any any
    |TTA|AGg|gct|gag|gaT|aCT|GCA|GtT|taT|taC|tgc|gct aRg nnk nnk nnk nnk nnk-
!
! W G Q G T T V T V S S (SEQ ID NO: 63)
      tgg ggc cag ggt act acG GTC ACC gtc tcc agt-3′ (SEQ ID NO: 64)
! BstEII...
(C3X5)5′-GCA|GtT|taT|taC|tgc|gct aRg nnk nnk nnk nnk nnk tgg-
                      ggc cag ggt act ac-3′ (SEQ ID NO: 65)
!-------------------------------------------------
aRg encodes K or R
```

Alternatively, the current HC diversity can be cloned into DY3F87HC and the CDR3 diversity described above is cloned into that diversity as XbaI-BstEII fragments. A library of, for example, 25 LC are cloned into pLCSK23 and used to create a cell line in TG1 E. coli. These cells are infected with the DY3F87HC phage which harbor the novel HC CDR3 (and CDR1-2) diversity. The phage obtained from this infection are selected for binding to a desired target. After two to four rounds of selection, the selected HCs are transferred to pHCSK22 and used to create a cell line which can be used with ROLIC to combine the selected HC with all the LCs in the ROLIC LC library. In this way, a library of 1. E 9 can be give Abs that normally would require construction of a library of 1. E 16 (assuming a LC diversity of 1. E 7).

### Prophetic Example 2: Libraries with Very Long HC CDR3s

Sidhu et al. (J Mol Biol. 2004 338:299-310. and U.S. application No. 20050119455A1) report high-affinity Abs selected from a library in which only Y and S were allowed in the CDRs which were limited in length to 20 amino acids. It may be possible to generate high affinity Abs from a library that has HC CDR3s with one or more of the following forms of diversity: a) several (but not all) sites allowing Y or S, b) including 4-6 NNK codons, c) introducing D segments (with or without diversification in the D), and/or d) using error-prone PCR. We have already sampled the Ab space in which HC CDR3 is in the range ˜8 to ˜22 with a median length of 13. Thus, libraries in which HC CDR3 is either ˜23 AAs or ˜35 AAs are possible and may have advantages with certain types of targets. For example, GPCRs are integral membrane proteins with seven helical segments transversing the lipid bilayer of the call that are thought to have multiple states. An antibody having a very long HC CDR3 could form a protuberance that fits into the channel formed by the seven strands. Finding Abs that bind GPCRs has been difficult and intentionally building libraries in which all the members have very long HC CDR3s may ameliorate this problem. The lengths may be made somewhat variable, say 23, 24, or 25 in one library and 33, 34, or 35 in a second.

Below are a number of representative designs. The CDR3 have been broken up and diversity generated that lets the various parts have differing relationships depending on the value of X. A full-length JH1 has been used, and in some designs diversity allowed diversity in the CDR3 part of JH1. Other JHs could be used. In the designs, the D segments are either rich in Y or have an S-rich disulfide loop. The amino-acid sequences of human D segments are shown in Table 3. The places where the D region has either S or Y or allowed other combinations have in particular been varied. Table 4 shows the amino-acid sequences of human J regions.

Each of the libraries could be built in at least four ways: 1) DNA encoding a particular amino acid sequence is first synthesized and subjected to error-prone PCR, 2) the library can be synthesized by wobbling or with mixtures of nucleotides, 3) the library can be built using dobbling, and 4) routes (2) or (3) could be followed by error-prone PCR. As an example of route (1), in Design 12, DNA encoding SEQ ID NO:908 could be synthesized, as shown in SEQ ID NO:911. This DNA could be subjected to error-prone PCR using the primers shown in SEQ ID NO:909 and SEQ ID NO:910. Because these primers cover the framework regions, the errors will occur only in the CDR3.

A library of HCs with CDR3 with length 23 of, for example, 2×10⁹ members and a second library with HC CDR3s of length ˜35 also having 2×10⁹ members could be built. Alternatively, the DNA could be mixed to build one library of 4×10⁹.

TABLE 4
Human JH amino-acid sequences

| | |
|---|---|
| | H3 |
| | ------ |
| | CDR3 |
| | --------- |
| | 100 110 |
| | \| \| |
| JH1 | ---AEYFQHWGQGTLVTVSS (SEQ ID NO: 66) |
| JH2 | ---YWYFDLWGRGTLVTVSS (SEQ ID NO: 67) |
| JH3 | -----AFDIWGQGTMVTVSS (SEQ ID NO: 2) |
| JH4 | -----YFDYWGQGTLVTVSS (SEQ ID NO: 1) |
| JH5 | ----NWFDPWGQGTLVTVSS (SEQ ID NO: 68) |
| JH6 | YYYYYGMDVWGQGTTVTVSS (SEQ ID NO: 3) |

In each of the following designs, the amino-acid sequence begins with YYCA (K/R) which is the end of FR3. FR4 starts with WG and is shown bold.

### Design 1

| XX::D2-2.2::XX::JH1 | |
|---|---|
| 1 1 2 2 | |
| FR3 1 5 0 5 0 3FR4 | |
| YYCAK DYGYCSSTSCYTKLYSYAEYFQHWGQGTLVTVSS (SEQ ID NO: 898) | |
| YYCAK XXGYCSXXSCYTXXYSYAEYFQHWGQGTLVTVSS (SEQ ID NO: 69) | |
| R GYCSSTSCYT AEYFQHWGQGTLVTVSS (JH1) | |
| (SEQ ID NO: 70) (SEQ ID NO: 66) | |
| 1 1 1 1 | |
| 9 9 0 0 0 1 | |
| 4 5 0 3abcdefghijklmn4 0 | |
| Amino-acid diversity | = 1.28 E 8 |
| DNA diversity | = 2.15 E 9 |
| Stop-free | = 83% |
| Gratuitous Cys-free | = 83% |
| Free of stop and Cys | = 68% |

Design 1 (C23D222) has 94 being R or K, then 2 Xs, D2-2 in second reading frame with two Xs in the loop, followed by two Xs, and JH1. D2-2 2^{nd} reading frame has a disulfide-closed loop into which diversity at two points has been introduced. This CDR3 is 23 long. Using primers that include DNA up to . . . YYCA and from WGQG . . . , error-prone PCR on the CDR3 could be performed before amplifying out to XbaI and BstEII for cloning into the library of kappa LC and HC CDR1/2. Thus, the AAs that are shown as fixed will be allowed to vary some. The AAs that are part of the PCR overlap region will be reinforced by the final non-error prone PCR. Error-prone PCR is not a necessary part of the design.

```
C23D222JH1
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3----------------------------------------------------------------
! X X D2-2 RF2............................. X X JH1..
! any any G Y C S any any S C Y T any any Y S Y A
      nnk nnk ggt tat tgt tcc nnk nnk tct tgc tat act nnk nnk tat tcc tac gct-
!
! CDR3---------------
! E Y F Q H
      gaa tat ttc cag cac-
!
! W G Q G T L V T V S S (SEQ ID NO: 71)
      tgg ggc cag ggt act ctG GTC ACC gtc tcc agt-3′ (SEQ ID NO: 72)
! BstEII...
(ON_C23D222) 5′-GCA|GtT|taT|taC|tgc|gct aRg nnk nnk ggt tat tgt tcc nnk-
            nnk tct tgc tat act nnk nnk tat tcc tac gct gaa tat ttc cag cac-
            tgg ggc cag ggt act ct-3′ ! 107 bases (SEQ ID NO: 73)
(ON_1) 5′-GCA|GtT|taT|taC|tgc|gct-3′ (SEQ ID NO: 74)
(ON_2) 5′-AgAgTAcccTggccccAgAcgTccATAccgTAATAgT-3′ ! 37 bases (SEQ ID NO: 75)
(ON_2 is reverse complement of 5′-ac tat tac ggt atg gac gtc tgg
ggc cag ggt act ct-3′) (SEQ ID NO: 76)
(ON_3) 5′-ttc|act|atc|TCT|AGA|gac|aac|tct|aag|aat|act|ctc|tac|ttg|cag|atg|-
                    aac|agC|TTA|AGg|gct|gag|gaT|aCT|GCA|GtT|taT|taC|tgc|gct-3′ (SEQ ID NO: 77)
(ON_4) 5′-AcTggAgAcggTgAccAgAgTAcccTggccccA-3′ ! 33 bases (SEQ ID NO: 78)
(5′-tgg ggc cag ggt act ctG GTC ACC gtc tcc agt-3′ [RC] (SEQ ID NO: 79))
```

### Design 2

| 1 1 2 2 | |
|---|---|
| 1 5 0 5 0 3 | |
| YYCAK GSYYYGSGSYYNMDSYYAEYFQHWGQGTLVTVSS (SEQ ID NO: 899) | |
| YYCAK XXYYYGXGSXYNXXSYYAEYFQHWGQGTLVTVSS (SEQ ID NO: 80) | |
| R YYYGSGSYYN AEYFQHWGQGTLVTVSS (JH1) | |
| (SEQ ID NO: 81) (SEQ ID NO: 66) | |
| Amino-acid diversity | = 1.28 E 8 |
| DNA diversity | = 2.15 E 9 |
| Stop-free | = 83% |
| Gratuitous Cys-free | = 83% |
| Free of stop and Cys | = 68% |

Design 2 (C23D310) has 94 as R or K, two Xs, D3-10 (RF2) with 5^{th} and 8^{th} residues changed to X, 2 Xs, SYY, and JH1. The CDR3 is 23 AA long and could be further diversified by use of error-prone PCR.

```
C23D310JH1
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3-------------------------------------------------------------------
!
! any any Y Y Y G any G S any Y N any any S Y Y
      nnk nnk tac tac tat ggt nnk ggc tct nnk tac aat nnk nnk tct tat tac
!
! A E Y F Q H
      gct gag tac ttt caa cat
!
! JH1......................................
! W G Q G T L V T V S S (SEQ ID NO: 82)
      tgg ggc cag ggt act ctG GTC ACC gtc tcc agt-3′ (SEQ ID NO: 83)
! BstEII...
(C23D310) 5′-GCA|GtT|taT|taC|tgc|gct aRg nnk nnk tac tac tat ggt nnk ggc-
  tct nnk tac aat nnk nnk tct tat tac gct gag tac ttt caa cat tgg ggc cag-
  ggt act ct-3′ (SEQ ID NO: 84)
ON_1, ON_2, ON_3, and ON_4 as above.
```

### Design 3

| 1 1 2 2 | |
|---|---|
| 1 5 0 5 0 3 | |
| YYCAK EYYYYGSGSYYNSTTTSAEYFQHWGQGTLVTVSS (SEQ ID NO: 900) | |
| YYCAK XZYZZGZGZXYNZXZYZAXZFQHWGQGTLVTVSS (SEQ ID NO: 84) | |
| R YYYGSGSYYN AEYFQHWGQGTLVTVSS (JH1) | |
| (SEQ ID NO: 81) (SEQ ID NO: 66) | |
| Amino-acid diversity | = 1.64 E 8 |
| DNA diversity | = 1.07 E 9 |
| Stop-free | = 88% |
| Gratuitous Cys-free | = 88% |
| Free of stop and Cys | = 77% |

Design 3 (C23D310B) has 94 as R or K, XZ, D3-10 (RE2) with 2^{nd}, 3^{rd}, 5^{th}, and 7^{th} as Z(V|S) and 8^{th} residue changed to X, ZXZYZ, and JH1 (with the E changed to X). Z is either Y or S.

The CDR3 is 23 AA long and could be further diversified by use of error-prone PCR.

```
                              A V Y Y C A R|K any Y|S Y Y|S Y|S G Y|S
(C23D310b) 5′-GCA|GtT|taT|taC|tgc|gct aRg nnk tmc tac tmc tmt ggt tmc ggc-
Y|S any Y N Y|S any Y|S Y Y|S A any Y|S F Q H W G Q
tmt nnk tac aat tmt nnk tmc tat tmc gct nnk tmc ttt caa cat tgg ggc cag-
  G T L (SEQ ID NO: 85)
ggt act ct-3′ (SEQ ID NO: 86)
ON_1, ON_2, ON_3, and ON_4 as above.
```

### Design 4

| 1 1 2 2 2 3 3 | |
|---|---|
| 1 5 0 5 0 3 5 0 5 | |
| YYCAK YYSFSYYPYYYDSSGYYYAYYSDYSYSYYAEYFQHWGQGTLVTVSS (SEQ ID NO: 901) | |
| YYCAK YYSXSYYXYZYDSZGYZYXYYSXYZYZZZAZZFQHWGQGTLVTVSS (SEQ ID NO: 87) | |
| R YYYDSSGYYY AEYFQHWGQGTLVTVSS (JH1) | |
| (SEQ ID NO: 88) (SEQ ID NO: 66) | |
| 1 1 1 1 | |
| 9 9 0 0 0 1 | |
| 4 5 0 3abcdefghijklmnopqrstuvwxya4 0 | |
| ′ | |
| Amino-acid diversity | = 1.64 E 8 |
| DNA diversity | = 1.07 E 9 |
| Stop-free | = 88% |
| Gratuitous Cys-free | = 88% |
| Free of stop and Cys | = 77% |

Design 4 has CDR3 of length 35. Residue 94 can be K or R, then YYS::X::SYY::X::D3-22 (2^{nd} RF with one S as X and 3 Zs)::X::YYS::X::YZZZ::JH1 (with 2 Zs). Error-prone PCR could be used to add more diversity.

```
C35D322JH1
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TAA|AGg|gct|gag|gaT|aCT|GCA|GtT|taT|taC|tgc|gct aRg -
!
! CDR3-------------------------------------------------------------------
!
! Y Y S any S Y Y any Y Y|S Y D S Y|S G Y Y|S Y
      tac tat tcc nnk tct tac tat nnk tat tmt tac gat agt tmt ggt tac tmc tat
!
      any Y Y S any Y Y|S Y Y|S Y|S Y|S A Y|S Y|S F Q H
      nnk tac tat agc nnk tat tmc tac tmc tmt tmc gct tmt tmc ttc caa cac
!
! W G Q G T L V T V S S (SEQ ID NO: 89)
      tgg ggc cag ggt act ctG GTC ACC gtc tcc agt-3′ (SEQ ID NO: 90)
! BstEII...
(c35d322B) 5′-GCA|GtT|taT|taC|tgc|gct aRg tac tat tcc nnk tct tac tat nnk-
    tat tmt tac gat agt tmt ggt tac tmc tat nnk tac tat agc nnk tat tmc tac-
    tmc tmt tmc gct tmt tmc ttc caa cac tgg ggc cag ggt act ct-3′ (SEQ ID NO: 91)
ON_1, ON_2, ON_3, and ON_4 as above.
```

### Design 5

| 1 1 2 2 | |
|---|---|
| 1 5 0 5 0 3 | |
| YYCAK SSGYCSSTSCYTNPYYYAEYFQHWGQGTLVTVSS (SEQ ID NO: 902) | |
| YYCAK ZZGZCZZXZCZTXXYZYXZYFQHWGQGTLVTVSS (SEQ ID NO: 92) | |
| R GYCSSTSCYT AEYFQHWGQGTLVTVSS (JH1) | |
| (SEQ ID NO: 70) (SEQ ID NO: 66) | |
| Amino-acid diversity | = 1.64 E 8 |
| DNA diversity | = 1.07 E 9 |
| Stop-free | = 88% |
| Gratuitous Cys-free | = 88% |
| Free of stop and Cys | = 77% |

Design 5 (C23D222b) is like design 1 but uses many Z(Y or S) variable codons. This CDR3 is 23 long.

```
C23D222JH1b
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3-------------------------------------------------------------------
! Y|S Y|S G Y|S C Y|S Y|S any Y|S C Y|S T any any Y Y|S Y any
      tmc tmt ggt tmt tgc tmc tmt nnk tmt tgt tmc acc nnk nnk tat tmt tac nnk
!
! Y|S Y F Q H
      tmt tat ttc cag cac
!
! W G Q G T L V T V S S (SEQ ID NO: 93)
      tgg ggc cag ggt act ctG GTC ACC gtc tcc agt-3′ (SEQ ID NO: 94)
! BstEII...
(C23D222JH1b) 5′-GCA|GtT|taT|taC|tgc|gct aRg tmc tmt ggt tmt tgc tmc tmt-
nnk tmt tgt tmc acc nnk nnk tat tmt tac nnk tmt tat ttc cag cac tgg ggc-
cag ggt act ct-3′ (SEQ ID NO: 95)
```

### Design 6

| 1 1 2 2 2 3 3 | |
|---|---|
| 1 5 0 5 0 3 5 0 5 | |
| YYCAK SYQYYGYCSSTSCYTYYSYWSYSSYYSYYAEYFQHWGQGTLVTVSS (SEQ ID NO: 903) | |
| YYCAK ZYXZYGZCZZXSCZTYZSZXZYSZYZSZYAEZFQHWGQGTLVTVSS (SEQ ID NO: 96) | |
| R GYCSSTSCYT D2-2.2 AEYFQHWGQGTLVTVSS (JH1) | |
| (SEQ ID NO: 70) (SEQ ID NO: 66) | |
| Amino-acid diversity | = 2.00 E 8 |
| DNA diversity | = 5.37 E 8 |
| Stop-free | = 91% |
| Gratuitous Cys-free | = 91% |
| Free of stop and Cys | = 83% |

```
C35D222JH1
!
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3-------------------------------------------------------------------
! Y|S Y any Y|S Y G Y|S C Y|S Y|S any S C Y|S T Y Y|S S
      tmt tac nnk tmc tac ggc tMt tgc tmt tmc nnk tCt tgt tmc acc tat tmt tcc
!
! Y|S any Y|S Y S any Y Y|S S Y|S Y A E Y F Q H
      tmt nnk tmc tat tct nnk tac tmc agt tmt tat gct gag tat ttc cag cac
!
! W G Q G T L V T V S S (SEQ ID NO: 97)
      tgg ggc cag ggt act ctG GTC ACC gtc tcc agt-3′ (SEQ ID NO: 98)
! BstEII...
(C35D222JH1) 5′-GCA|GtT|taT|taC|tgc|gct aRg tmt tac nnk tmc tac ggc tat- tgc tmt tmc
nnk tmt tgt tmc acc tat tmt tcc tmt nnk tmc tat tct nnk tac-
tmc agt tmt tat gct gag tat ttc cag cac tgg ggc cag ggt act ct-3′ (SEQ ID NO: 99)
```

### Design 7

| 1 1 2 2 2 3 3 | |
|---|---|
| 1 5 0 5 0 3 5 0 5 | |
| YYCAK YYSYYGYCSSTSCYTYSSSPSYSYYSSYYAEYFQHWGQGTLVTVSS (SEQ ID NO: 904) | |
| YYCAK ZYZZYGZCZZXZCZTYZSZXZYSZYZSZYAψZJQBWGQGTLVTVSS (SEQ ID NO: 100) | |
| R GYCSSTSCYT D2-2.2 AEYFQHWGQGTLVTVSS (JH1) | |
| (SEQ ID NO: 70) (SEQ ID NO: 66) | |
| (J = FSY, B = YHND, ψ = EKQ) | |
| Amino-acid diversity | = 9.44 E 8 |
| DNA diversity | = 2.42 E 9 |
| Stop-free | = 93% |
| Gratuitous Cys-free | = 93% |
| Free of stop and Cys | = 88% |

```
C35D222JH1B
!
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3----------------------------------------------------------------
! Y|S Y Y|S Y|S Y G Y|S C Y|S Y|S any Y|S C Y|S T Y Y|S S
      tmt tac tmc tmc tac ggc tMt tgc tmt tmc nnk tmt tgt tmc acc tat tmt tcc
!
! Q Y N|D
! Y|S any Y|S Y S Y|S Y Y|S S Y|S Y A E|K Y|S F|S Q H|Y
      tmt nnk tmc tat tct tmt tac tmc agt tmt tat gct Vag tmt tHc cag Nac
!
! W G Q G T L V T V S S (SEQ ID NO: 101)
      tgg ggc cag ggt act ctG GTC ACC gtc tcc agt-3′ (SEQ ID NO: 102)
! BstEII...
```

### Design 8

| 1 1 2 2 2 3 3 | |
|---|---|
| 1 5 0 5 0 3 5 0 5 | |
| YYCAK SPSYYDYVWGSYRYTSSYTYYSYSYSSYAEYFQHWGQGTLVTVSS (SEQ ID NO: 905) | |
| YYCAK ZXZYZBZVWGZZRZTZSZXZYZZZYZSZAψZFQHWGQGTLVTVSS (SEQ ID NO: 103) | |
| R YYDYVWGSYRYT D3-16.2 AEYFQHWGQGTLVTVSS (JH1) | |
| (SEQ ID NO: 104) (SEQ ID NO: 66) | |
| (J = FSY, B = YHND, ψ = EKQ) | |
| Amino-acid diversity | = 9.44 E 8 |
| DNA diversity | = 1.61 E 9 |
| Stop-free | = 93% |
| Gratuitous Cys-free | = 93% |
| Free of stop and Cys | = 88% |

```
C34D316JH1A
!
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3---------------------------------------------------------------
! N|D
! Y|S any Y|S Y Y|S Y|H Y|S V W G Y|S Y|S R Y|S T Y|S
      tmt nnk tmc tac tmt Nat tmt gtt tgg ggt tmt tmc cgt tmt act tmt
!
! S Y|S any Y|S Y Y|S Y|S Y|S Y Y|S S Y|S
      agt tmc nnk tmt tac tmc tmt tmc tat tmc agt tmt
!
! Q
! A E|K Y|S F Q H
      GCT vag tmc ttc cag cat
!
! W G Q G T L V T V S S (SEQ ID NO: 105)
      tgg ggc cag ggt act ctG GTC ACC gtc tcc agt-3′ (SEQ ID NO: 106)
! BstEII...
(C34D316JH1A) 5′-GCA|GtT|taT|taC|tgc|gct aRg tmt nnk tmc tac tmt Nat tmt-
gtt tgg ggt tmt tmc cgt tmt act tmt agt tmc nnk tmt tac tmc tmt tmc tat-
tmc agt tmt GCT vag tmc ttc cag cat tgg ggc cag ggt act ct -3′ (SEQ ID NO: 107)
```

### Design 9

Design 9 is like 8 except the D segment is moved to the right

| 1 1 2 2 2 3 3 | |
|---|---|
| 1 5 0 5 0 3 5 0 5 | |
| YYCAK YAYSSESYYSSYYDYVWGSYRYTYSSYYAEYFQHWGQGTLVTVSS (SEQ ID NO: 906) | |
| YYCAK ZXZZZXZYZZZYZBZVWGZZRZTYZSZYAψZFQHWGQGTLVTVSS (SEQ ID NO: 108) | |
| R D3-16.2 YYDYVWGSYRYT AEYFQHWGQGTLVTVSS (JH1) | |
| (SEQ ID NO: 104) (SEQ ID NO: 66) | |
| (J = FSY, B = YHND, ψ = EKQ) | |
| Amino-acid diversity | = 1.31 E 8 |
| DNA diversity | = 5.37 E 8 |
| Stop-free | = 91% |
| Gratuitous Cys-free | = 91% |
| Free of stop and Cys | = 83% |

```
C34D316JH1B
!
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI...
!
! L R A E D T A V Y Y C A K|R
    |TTA|AGg|gct|gag|gaT|aCT|GCA|GtT|taT|taC|tgc|gct aRg -
!
! CDR3--------------------------------------------------------------------
! Y|S any Y|S Y|S Y|S any Y|S Y Y|S Y|S Y|S
      tmt nnk tmc tmt tmc nnk tmt tac tmc tmt tmc
!
! N|D
! Y Y|S Y|H Y|S V W G Y|S Y|S R Y|S T
      tac tmt Nat tmt gtt tgg ggt tmt tmc cgt tmt act
!
! Y Y|S S Y|S Y
      tat tmc agt tmt tac
!
! Q
! A E|K Y|S F Q H
      GCT vag tmc ttc cag cat
!
! W G Q G T L V T V S S (SEQ ID NO: 109)
      tgg ggc cag ggt act ctG GTC ACC gtc tcc agt-3′ (SEQ ID NO: 110)
! BstEII...
(C35D316JH1B)
5′-GCA|GtT|taT|taC|tgc|gct aRg tmt nnk tmc tmt tmc nnk tmt tac tmc tmt tmc
tac tmt Nat tmt gtt tgg ggt tmt tmc cgt tmt act tat tmc agt tmt tac GCT vag
tmc ttc cag cat tgg ggc cag ggt act ct-3′ (SEQ ID NO: 111)
```

### Design 10

```
                              1 1 2 2
            1 5 0 5 0 4
YYCAK GSSYYYGSGSYYNSEYYSAEYFQHWGQGTLVTVSS (SEQ ID NO: 907)
YYCAK XZZYZZGZGZXYNZXZYZAXZFQHWGQGTLVTVSS (SEQ ID NO: 112)
        R YYYGSGSYYN AEYFQHWGQGTLVTVSS (JH1)
                (SEQ ID NO: 81) (SEQ ID NO: 66)
```

Design 10 (C24D310B) is like Design 3, but the CDR3 is of length 24. Design 10 has 94 as R or K, XZ, D3-10 (RF2) with 2^{nd}, 3^{rd}, 5^{th}, and 7^{th} as Z(Y|S) and 8^{th} residue changed to X, ZXZYZ, and JH1 (with the E changed to X). Z is either Y or S. The CDR3 is 24 AA long and could be further diversified by use of error-prone PCR.

(C24D310b)
(SEQ ID NO: 113)
5′-GCA|GtT|taT|taC|tgc|gct aRg nnk tmc tmc tac tmc tmt ggt tmc-
ggc tmt nnk tac aat tmt nnk tmc tat tmc gct nnk tmc ttt caa cat tgg ggc-
cag ggt act ct-3′
ON_1, ON_2, ON_3, and ON_4 as above.

### Design 11

```
                              1 1 2 2
            1 5 0 5 0 5
YYCAR SSRSGYCTNGVCYTSKSYWYFDLWGRGTLVTVSS (SEQ ID NO: 907)
YYCAR ZZXZGZC32GVCZ3ZXZZ4Z12LWGRGTLVTVSS (SEQ ID NO: 114)
        K GYCTNGVCYT YWYFDLWGRGTLVTVSS D2-8.2 JH2
                  (SEQ ID NO: 115) (SEQ ID NO: 67)
(1 = FYS(THT), 2 = YHND(NAT), 3 = ITKR(ANA), 4 = LSW(TBG))
(C24D282)
(SEQ ID NO: 116)
5′-GCA|GtT|taT|taC|tgc|gct aRg tmc tmt nnk tmt ggt tmc tgt ana-
nat ggt gtc tgc tmt ana tmc nnk tmt tmt tbg tmt tht nat ctg tgg ggc-
cag ggt act ct-3′
(C24D282.1)
(SEQ ID NO: 117)
5′-GCA|GtT|taT|taC|tgc|gct aRg tmc tmt nnk tmc ggt tmc tgc ana-
nat ggc gtc tgc tmt ana tmc nnk tmt tmt tbg tmt tht nat ctg tgg ggc-
cag ggt act ct-3′
(C24D282.1)
(SEQ ID NO: 118)
5′-GCA|GtT|taT|taC|tgc|gct aRg tmc tmt nnk tmc ggt tmc tgc ana-
nat ggc gtc tgc t-3′ (needs R, M, N, K)
(C24D282.2)
(SEQ ID NO: 119)
5′-Ag AgT Acc cTg gcc ccA cAg ATN ADA AKA cVA AKA AKA MNN gKA TNT
AKA gcA gAc gcc ATN TNT gcA gKA Acc g-3′ ! 75 bases
(5′-c ggt tmc tgc ana-
nat ggc gtc tgc tmt ana tmc nnk tmt tmt tbg tmt tht nat ctg tgg ggc-
cag ggt act ct-3′ [RC] (SEQ ID NO: 120) (needs N, M, K, B, H))
```

### Design 12

```
                              1 1 2 2 3 3
            1 5 0 5 0 5 0 5
YYCAR SSYYSYGYCTNGVCYTYSYSYYSYSYSYWYFDLWGRGTLVTVSS (SEQ ID NO: 908)
YYCAR ZZZZZZGZC32GVCZ3ZZZZYZZYZYZZ4Z12LWGRGTLVTVSS (SEQ ID NO: 121)
        K GYCTNGVCYT YWYFDLWGRGTLVTVSS D2-8.2 JH2
                      (SEQ ID NO: 115) (SEQ ID NO: 67)
(1 = FYS, 2 = YHND, 3 = ITKR, 4 = LSW, Z = YS)
(C33D282TP)
(SEQ ID NO: 909)
5′-GCA|GtT|taT|taC|tgc|gct-3′
(C33D282BP)
(SEQ ID NO: 910)
5′-ag agt acc ctg gcc cca-3′
(C33D282)
(SEQ ID NO: 122)
5′-GCA|GtT|taT|taC|tgc|gct aRg tmt tmc tmc tmt tmc tmc ggt-
tmt tgt ana nat ggc gtg tgc tmt ana tmc tmc tmc tmt tat tmt tmc tat tmt-
tac tmt tmc tbg tmc tht nat ctg tgg ggc cag ggt act ct-3′
(C33D282F)
(SEQ ID NO: 911)
5′-GCA|GtT|taT|taC|tgc|gct agg tct tcc tac tat tcc tac ggt-
tat tgt aca aat ggc gtg tgc tat aca tac tcc tac tct tat tat tcc tat tct-
tac tct tac tgg tac ttt gat ctg tgg ggc cag ggt act ct-3′
```

### Design 13

Design 13 places a germ-line D segment in the middle of a sea of Zs so that one can make two pieces of DNA that overlap throughout the constant region. HC CDR3 is 34 long and diversity is 2²³˜8×10⁶.

```
                              1 1 2 2 3 3
            1 5 0 5 0 5 0 5
YYCAR SSSYYSYYSSGYCTNGVCYTYSSYYSSYYWYFDLWGRGTLVTVSS (SEQ ID NO: 912)
YYCAR ZZZZZZZZZZGYCTNGVCYTZZZZZZZZZWZF2LWGRGTLVTVSS (SEQ ID NO: 123)
        K GYCTNGVCYT YWYFDLWGRGTLVTVSS D2-8.2 JH2
                              (SEQ ID NO: 115) (SEQ ID NO: 67)
(2 = YHND)
(C340282.2A)
(SEQ ID NO: 124)
5′-GCA|GtT|taT|taC|tgc|gct aRg tmt tmc tmc tmt tmt tmc tmc tmt-
tmc tmc ggt tat tgt act aac ggc gtt tgc tat act-3′
(C340282.2B)
(SEQ ID NO: 125)
5′-Ag AgT Acc cTg gcc ccA cAg gTN gAA AKA ccA AKA AKA AKA gKA-
gKA gKA gKA AKA AKA AgT ATA gcA AAc gcc gTT AgT AcA ATA-3′ ! 86 bases
(5′- tat tgt act aac ggc gtt tgc tat act tmt tmt tmc tmc tmc tmc-
tmt tmt tmt tgg tmt ttc Nac ctg tgg ggc cag ggt act ct-3′ (SEQ ID NO: 126) [RC])
```

### Design 14

Design 14 is like 9 except the D segment is mostly germline.

```
                              1 1 2 2 2 3 3
            1 5 0 5 0 3 5 0 5
YYCAK YSYYSSSYYYSDYVWGSYRYTSYYSYYYAEYFQHWGQGTLVTVSS (SEQ ID NO: 913)
YYCAK ZZZZZZZZZZZDYVWGSYRZTZZZZZZZAEZFQHWGQGTLVTVSS (SEQ ID NO: 127)
        R D3-16.2 YYDYVWGSYRYT AEYFQHWGQGTLVTVSS (JH1)
                            (SEQ ID NO: 104) (SEQ ID NO: 66)
(C34D316.2A)
(SEQ ID NO: 128)
5′-GCA|GtT|taT|taC|tgc|gct aRg tmt tmc tmc tmt tmt tmc tmc tmt-
tmc tmc tmc gat tat gtc tgg ggt act tat cgt-3′
(C34D316.2B)
(SEQ ID NO: 129)
5′-Ag AgT Acc cTg gcc ccA ATg cTg gAA AKA cTc Agc gKA gKA gKA-
gKA gKA gKA AKA AgT gKA Acg ATA AgT Acc ccA gAc ATA ATc-3′ ! 86 bases
(5′-gat tat gtc tgg ggt act tat cgt tmc act tmt tmc tmc tmc tmc-
tmc tmc gct gag tmt ttc cag cat tgg ggc cag ggt act ct-3′ (SEQ ID NO: 130) [RC])
```

### Design 15

Design 15 allows some diversity in the overlap, 5 two-way flip-flops. There are only 32 overlap sequences and even if there are mismatches, they will not change the allowed diversity.

```
                              1 1 2 2 2 3 3
            1 5 0 5 0 3 5 0 5
YYCAK SYYYSSYSYYYDYVWGSYRYTSYSSSSYYAEYFQHWGQGTLVTVSS (SEQ ID NO: 914)
YYCAK ZZZZZZZZZZZDZVWGZZRZTZZZZZZZZAEZFQHWGQGTLVTVSS (SEQ ID NO: 131)
                              YYDYVWGSYRYT AEYFQHWGQGTLVTVSS
                                  (SEQ ID NO: 104) (SEQ ID NO: 66)
(C35D316.2A)
(SEQ ID NO: 132)
5′-GCA|GtT|taT|taC|tgc|gct aRg tmt tmc tmc tmt tmt tmc tmc tmt-
tmc tmc tmc gac tmt gtc tgg ggt tmc tmc cgt tmc acc t-3′
(C35D316.2B)
(SEQ ID NO: 133)
5′-Ag AgT Acc cTg gcc ccA ATg cTg gAA AKA cTc Agc gKA gKA-
gKA gKA gKA gKA gKA AKA ggT gKA Acg gKA gKA Acc ccA gAc AKA gTc gKA g-3′
(5′-c tmc gac tmt gtc tgg ggt tmc tmc cgt tmc acc tmt tmc tmc-
tmc tmc tmc tmc tmc gct gag tmt ttc cag cat tgg ggc cag ggt act ct-3′
(SEQ ID NO: 134) [RC])
```

### Design 16

Design 16 provides a CDR3 of 35. There are 4 two-way flip-flops in the overlap, thus 16 sequences.

```
                              1 1 2 2 2 3 3
            1 5 0 5 0 3 5 0 5
YYCAK SSSYYSYSYSGYCSGGSCYSSYYYSSYYSAEYFQGWGQGTLVTVSS (SEQ ID NO: 915)
YYCAK ZZZZZZZZZZGZCZGGZCZSZZZZZZZZZAEZFQHWGQGTLVTVSS (SEQ ID NO: 135)
        R GYCSGGSCYS 2-25.2 AEYFQHWGQGTLVTVSSJH1
                                (SEQ ID NO: 136) (SEQ ID NO: 66)
(C35D225.2A) 5′-GCA|GtT|taT|taC|tgc|gct aRg tmt tmt tmt tmt tmt tmt tmt tmt-
              tmc tmc ggc tmc tgt tmc ggt ggc tmc tgc tmc tcc t-3′ (SEQ ID NO: 137)
(C35D225.2B) 5′-Ag AgT Acc cTg gcc ccA ATg TTg gAA AKA TTc Agc gKA gKA-
      gKA gKA gKA gKA gKA gKA gKA gKA ggA gcA gKA gcc Acc gKA AcA gKA gcc gKA g-3′
    (SEQ ID NO: 138) ! 96 bases
```

If we add C34D) 225.2.A and C341) 225.2B to the mixture, then we get CDR3s of lengths 33, 34, and 35.

(C34D225.2A)
(SEQ ID NO: 139)
5′-GCA|GtT|taT|taC|tgc|gct aRg tmt tmt
tmt tmt tmt tmt tmt-tmc tmc ggc tmc tgt tmc ggt
ggc tmc tgc tmc tcc t-3′
(C34D225.2B)
(SEQ ID NO: 140)
5′-Ag AgT Acc cTg gcc ccA ATg TTg gAA AKA TTc
Agc gKA gKA-gKA gKA gKA gKA gKA gKA gKA ggA
gcA gKA gcc Acc gKA AcA gKA gcc gKA g-3′ !
93 bases

### Design 17

```
                              1 1 2 2 2 3 3
            1 5 0 5 0 3 5 0 5
YYCAK YSSYSYYDYVWGSYRYTSSSYSYYSYYYAEYFQGWGQGTLVTVSS (SEQ ID NO: 916)
YYCAK ZZZZZZZDZVWGZZRZTZZZZZZZZZZZAEZFQHWGQGTLVTVSS (SEQ ID NO: 141)
        R YYDYVWGSYRYT D3-16.2 AEYFQHWGQGTLVTVSS (JH1)
                    (SEQ ID NO: 104) (SEQ ID NO: 66)
(C35D3162A)
(SEQ ID NO: 142)
5′-GCA|GtT|taT|taC|tgc|gct aRg tmt tmt tmt tmt tmt tmt tmc gac-
tmc gtc tgg ggt tmt tmc cgt tmt acc t-3′
(C35D3162B)
(SEQ ID NO: 143)
5′-Ag AgT Acc cTg gcc ccA gTg cTg gAA gKA cTc Agc gKA gKA gKA-
gKA gKA gKA gKA gKA gKA gKA gKA gKA gKA ggT AKA Acg gKA AKA Acc ccA gAc-
gKA gTc g-3′
```

### Design 18

```
                              1 1 2 2 2 3 3
            1 5 0 5 0 3 5 0 5
YYCAK SSYYYSSSYYDYVWGSYRYTSSYYSYSYAEYFQGWGQGTLVTVSS (SEQ ID NO: 917)
YYCAK ZZZZZZZZZZDZVWGZZRZTZZZZZZZZAEZFQHWGQGTLVTVSS (SEQ ID NO: 144)
        R YYDYVWGSYRYT D3-16.2AEYFQHWGQGTLVTVSS (JH1)
                            (SEQ ID NO: 104) (SEQ ID NO: 66)
(C35D3162C)
(SEQ ID NO: 145)
5′-GCA|GtT|taT|taC|tgc|gct aRg tmt tmt tmt tmt tmt tmt tmc-
tmc tmc tmc gac tmc gtc tgg ggt tmc tmc cgt tmc acc t-3′ 82 bases
(C35D3162B)
(SEQ ID NO: 146)
5′-Ag AgT Acc cTg gcc ccA gTg cTg gAA gKA cTc Agc gKA gKA-
gKA gKA gKA gKA gKA gKA gKA gKA ggT gKA Acg gKA gKA Acc ccA gAc gKA-
gTc g-3′
```

### Design 19

```
                              1 1 2 2 2 3 3
            1 5 0 5 0 3 5 0 5
YYCAK YSSSSYSYYYYDSSGYYYSYYSSSYYSYYAEYFQGWGQGTLVTVSS (SEQ ID NO: 918)
YYCAK ZZZZZZZZZZZDSSGZZZZZZZZZZZZZZAEZFQHWGQGTLVTVSS (SEQ ID NO: 147)
        R YYYDSSGYYY AEYFQHWGQGTLVTVSS (JH1)
                                  (SEQ ID NO: 88) (SEQ ID NO: 66)
                      1 1 1 1
        9 9 0 0 0 1
        4 5 0 3abcdefghijklmnopqrstuvwxya4 0
                                                                                ′
Amino-acid diversity = 6.7 E 7
DNA diversity = 6.7 E 7
Stop-free = 100
Gratuitous Cys-free = 100
Free of stop and Cys = 100%
```

Design 19 has CDR3 of length 35. Residue 94 can be K or R. The ZZZZZZZZZ::D3-22 (2^{nd} RF with six Ys as Z)::ZZZZZZZZZZZ::JH1 (with 1 Z). Error-prone PCR could be used to add more diversity.

```
C35D322AJH1
! scab DNA S R D N S K N T L Y L Q M N S
5′-ttc|act|atc|TCT|AGA|gac|aac|tct|aag|aat|act|ctc|tac|ttg|cag|atg|aac|agC-
! XbaI . . .
!
! L R A E D T A V Y Y C A K|R
    |TTA|AGg|gct|gag|gaT|aCT|GCA|GtT|taT|taC|tgc|gct aRg-
!
! CDR3-------------------------------------------------------------------
!
! Y|S Y|S Y|S Y|S Y|S Y|S Y|S Y|S Y|S Y|S Y|S D S S G Y|S Y|S Y|S
      tmc tmt tmc tmc tmt tmc tmt tmc tmc tmc tmc gac agc tcc ggc tmc tmc tmt
!
      Y|S Y|S Y|S Y|S Y|S Y|S Y|S Y|S Y|S Y|S Y|S A E Y|S F Q H
      tmc tmt tmc tmc tmt tmc tmt tmc tmc tmc tmc gct gaa tmc ttc caa cac
!
! W G Q G T L V T V S S (SEQ ID NO: 148)
      tgg ggc cag ggt act ctG GTC ACC gtc tcc agt-3′ (SEQ ID NO: 149)
! BstEII . . .
(C35D322AJH1_T)
(SEQ ID NO: 150)
5′-GCA|GtT|taT|taC|tgc|gct aRg tmc tmt tmc tmc tmt-
tmc tmt tmc tmc tmc tmc gac agc tcc ggc tmc tmc t-3′
(C35D322AJH1_13)
(SEQ ID NO: 151)
5′-cAg AgT Acc cTg gcc ccA gTg TTg gAA gKA TTc Agc gKA-
gKA gKA gKA AKA gKA AKA gKA gKA AKA gKA AKA gKA gKA gcc ggA gcT gTc-
gKA gKA g-3′
ON_1, ON_2, ON_3, and ON_4 as above.
```

### Design 20

```
                                  1 1 2 2 2 3 3
            1 5 0 5 0 3 5 0 5
YYCAK YSSYSS YYYYDSSGYYYSSYSSYS YYYAEYFQGWGQGTLVTVSS (SEQ ID NO: 919)
YYCAK ZZZZZZ(Z)ZZZZDSSGZZZZZZZZZZ(Z)ZZZAEZFQHWGQGTLVTVSS (SEQ ID NO: 152)
        R YYYDSSGYYY AEYFQHWGQGTLVTVSS (JH1)
                            (SEQ ID NO: 88) (SEQ ID NO: 66)
                      1 1 1 1
        9 9 0 0 0 1
        4 5 0 3abcdefghijklmnop q rstuvwxya4 0
                                                                                          ′
Amino-acid diversity = 6.7 E 7
DNA diversity = 6.7 E 7
Stop-free = 100
Gratuitous Cys-free = 100
Free of stop and Cys = 100%
```

Design 20 has CDR3s of length 33, 34, or 35. Residue 94 can be K or R, The ZZZZZZ(Z)ZZ::D3-22 (2^{nd} RF with six Ys as Z)::ZZZZZZZ(Z)ZZZ::JH1 (with 1 Z). PCR combining (C35D322AJH1_T), (C34D322AJH1_T), (C35D322AJH1_B), and (C34D322AJH1_B) allows length as well as sequence diversity.

(C35D322AJH1_T)
(SEQ ID NO: 153)
5′-GCA|GtT|taT|taC|tgc|gct aRg tmc tmt tmc tmc-
tmt tmc tmt tmc tmc tmc tmc gac agc tcc ggc tmc tmc t-3′
(C34D322AJH1_T)
(SEQ ID NO: 154)
5′-GCA|GtT|taT|taC|tgc|gct aRg tmc tmc tmc tmt-
tmc tmt tmc tmc tmc tmc gac agc tcc ggc tmc tmc t-3′
(C35D322AJH1_B)
(SEQ ID NO: 920)
5′-cAg AgT Acc cTg gcc ccA gTg TTg gAA gKA TTc Agc gKA-
gKA gKA gKA AKA gKA AKA gKA gKA AKA gKA AKA gKA gKA gcc ggA gcT gTc-
gKA gKA g-3′
(C34D322AJH1_B)
(SEQ ID NO: 155)
5′-cAg AgT Acc cTg gcc ccA gTg TTg gAA gKA TTc Agc gKA-
gKA gKA gKA AKA gKA AKA gKA gKA AKA AKA gKA gKA gcc ggA gcT gTc-
gKA gKA g-3′

### Selection Against Stop Codons:

Because some of these libraries have NNK codons, they will have some TAG stop codons. We could remove the clones with TAG by cloning the amplified DNA into an XbaI-BstEII site between the signal sequence for a bla gene and the actual bla protein and express in Sup⁰ cells. Bla^{R} colonies do not contain TAG stops. Alternatively, we could clone the XbaI-BstEII fragments ahead of a kanamycin-resistance gene and select for Kan^{R}. We would then move the XbaI-BstEII cassette into the phage library.

TABLE 20
Human D regions
!* for TAG; @ for TAA; $ for TGA
D-Amino acid sequence alignment (RF: reading frame)

| | | RF 1 | RF 2 | RF 3 | Used in designs |
|---|---|---|---|---|---|
| D1 | 1-1 | (SEQ ID NO: 156) | (SEQ ID NO: 157) | (SEQ ID NO: 158) | |
| | | GTTGT | VQLER | YNWND | |
| | 1-7 | (SEQ ID NO: 159) | (SEQ ID NO: 160) | (SEQ ID NO: 161) | |
| | | GITGT | V*LEL | YNWNY | |
| | 1-20 | (SEQ ID NO: 159) | (SEQ ID NO: 162) | (SEQ ID NO: 163) | |
| | | GITGT | V*LER | YNWND | |
| | 1-26 | (SEQ ID NO: 164) | (SEQ ID NO: 165) | (SEQ ID NO: 166) | |
| | | GIVGAT | V*WELL | YSGSYY | |
| D2 | 2-2 | (SEQ ID NO: 167) | (SEQ ID NO: 70) | (SEQ ID NO: 168) | 1, 5, 6, 7, |
| | | RIL**YQLLY | GYCSSTSCYT | DIVVVPAAI | |
| | 2-8 | (SEQ ID NO: 169) | (SEQ ID NO: 115) | (SEQ ID NO: 170) | 20, 21, 22, |
| | | RILY@WCMLY | GYCTNGVCYT | DIVLMVYAI | |
| | 2-15 | (SEQ ID NO: 171) | (SEQ ID NO: 136) | (SEQ ID NO: 172) | 25, |
| | | RIL*WW*LLL | GYCSGGSCYS | DIVVVVAAT | |
| | 2-21 | (SEQ ID NO: 173) | (SEQ ID NO: 174) | (SEQ ID NO: 175) | |
| | | SILWW$LLF | AYCGGDCYS | HIVVVTAI | |
| D3 | 3-3 | (SEQ ID NO: 176) | (SEQ ID NO: 177) | (SEQ ID NO: 178) | |
| | | VLRFLEWLLY | YYDFWSGYYT | ITIFGVVII | |
| | 3-9 | (SEQ ID NO: 179) | (SEQ ID NO: 180) | (SEQ ID NO: 181) | |
| | | VLRYFDWLL@ | YYDILTGYYN | ITIF*LVII | |
| | 3-10 | (SEQ ID NO: 182) | (SEQ ID NO: 81) | (SEQ ID NO: 183) | |
| | | VLLWFGELL@ | YYYGSGSYYN | ITMVRGVII | |
| | 3-16 | (SEQ ID NO: 184) | (SEQ ID NO: 104) | (SEQ ID NO: 185) | 8, 9, 14, 15, 17, 18 |
| | | VL$LRLGELSLY | YYDYVWGSYRYT | IMITFGGVIVI | |
| | 3-22 | (SEQ ID NO: 186) | (SEQ ID NO: 187) | (SEQ ID NO: 188) | 4, 19, 20 |
| | | VLL***WLLL | YYYDSSGYYY | ITMIVVVIT | |
| D4 | 4-4 | (SEQ ID NO: 189) | (SEQ ID NO: 88) | (SEQ ID NO: 190) | |
| | | $LQ@L | DYSNY | TTVT | |
| | 4-11 | (SEQ ID NO: 191) | (SEQ ID NO: 192) | (SEQ ID NO: 193) | |
| | | $LQ@L | DYSNY | TTVT | |
| | 4-17 | (SEQ ID NO: 194) | (SEQ ID NO: 195) | (SEQ ID NO: 196) | |
| | | $LR@L | DYGDY | TTVT | |
| | 4-23 | (SEQ ID NO: 197) | (SEQ ID NO: 198) | (SEQ ID NO: 199) | |
| | | $LRW@L | DYGGNS | TTVVT | |
| D5 | 5-5 | (SEQ ID NO: 200) | (SEQ ID NO: 201) | (SEQ ID NO: 202) | |
| | | VDTAMV | WIQLWL | GYSYGY | |
| | 5-12 | (SEQ ID NO: 203) | (SEQ ID NO: 204) | (SEQ ID NO: 205) | |
| | | VDIVATI | WI*WLRL | GYSGYDY | |
| | 5-18 | (SEQ ID NO: 206) | (SEQ ID NO: 207) | (SEQ ID NO: 208) | |
| | | VDTAMV | WIQLWL | GYSYGY | |
| | 5-24 | (SEQ ID NO: 209) | (SEQ ID NO: 210) | (SEQ ID NO: 211) | |
| | | VEMATI | *RWLQL | RDGYNY | |
| D6 | 6-6 | (SEQ ID NO: 212) | (SEQ ID NO: 213) | (SEQ ID NO: 214) | |
| | | EYSSSS | SIAAR | V*QLV | |
| | 6-13 | (SEQ ID NO: 215) | (SEQ ID NO: 216) | (SEQ ID NO: 217) | |
| | | GYSSSWY | GIAAAG | V*QQLV | |
| | 6-19 | (SEQ ID NO: 218) | (SEQ ID NO: 219) | (SEQ ID NO: 220) | |
| | | GYSSGWY | GIAVAG | V*QWLV | |
| D7 | 7-27 | (SEQ ID NO: 221) | (SEQ ID NO: 222) | (SEQ ID NO: 223) | |
| | | LTG | @LG | NWG | |

TABLE 3
Human JH segments

JH-Amino acid sequence alignment

| | H3 |
|---|---|
| | ------ |
| | CDR3 |
| | -------- |
| | 100 110 |
| | \| FR4-------- Used in examples |
| JH1 | ---AEYFQHWGQGTLVTVSS 1-8, (SEQ ID NO: 66) |
| JH2 | ---YWYFDLWGRGTLVTVSS (SEQ ID NO: 67) |
| JH3 | -----AFDIWGQGTMVTVSS (SEQ ID NO: 2) |
| JH4 | -----YFDYWGQGTLVTVSS (SEQ ID NO: 1) |
| JH5 | ----NWFDPWGQGTLVTVSS (SEQ ID NO: 68) |
| JH6 | YYYYYGMDVWGQGTTVTVSS (SEQ ID NO: 3) |
| | 123456 |

TABLE 10
DNA encoding V-5D2-8.2a-JH2 for wobbling

```
! CDR3.......
! A E D T A V Y Y C A K D I V L M
    |gct|gag|gaT|aCT|GCA|GtT|taT|taC|tgc|gct aag jez ezq jzz qzz ezj
!
! W G Q G T T V T V S S (SEQ ID NO: 224)
        tgg ggc cag ggt act acG GTC ACC gtc tcc agt-3′ (SEQ ID NO: 225)
! BstEII...
```

TABLE 11
Trimers that can be extracted from human D segments
In Tables 11-14, the use of a lower case letter in
an amino acid sequence indicates that a stop codon
was changed to the residue listed as the lower case
letter. For example, in the amino acid sequence
“yLE”, a Tyr residue was introduced in place of a
stop codon.

| | | | |
|---|---|---|---|
| GTT | D1-1.1.1 | 1 | |
| VQL | D1-1.2.1 | 2 | |
| YNW | D1-1.3.1 | 3 | |
| TTG | D1-1.1.2 | 4 | |
| QLE | D1-1.2.2 | 5 | |
| NWN | D1-1.3.2 | 6 | |
| TGT | D1-1.1.3 | 7 | |
| LER | D1-1.2.3 | 8 | |
| WND | D1-1.3.3 | 9 | |
| GIT | D1-7.1.1 | 10 | |
| VyL | D1-7.2.1 | 11 | * |
| ITG | D1-7.1.2 | 12 | |
| yLE | D1-7.2.2 | 13 | * |
| LEL | D1-7.2.3 | 14 | |
| WNY | D1-7.3.3 | 15 | |
| GIV | D1-26.1.1 | 16 | |
| VyW | D1-26.2.1 | 17 | * |
| YSG | D1-26.3.1 | 18 | |
| IVG | D1-26.1.2 | 19 | |
| yWE | D1-26.2.2 | 20 | * |
| SGS | D1-26.3.2 | 21 | |
| VGA | D1-26.1.3 | 22 | |
| WEL | D1-26.2.3 | 23 | |
| GSY | D1-26.3.3 | 24 | |
| GAT | D1-26.1.4 | 25 | |
| ELL | D1-26.2.4 | 26 | |
| SYY | D1-26.3.4 | 27 | |
| RIL | D2-2.1.1 | 28 | |
| GYC | D2-2.2.1 | 29 | # |
| DIV | D2-2.3.1 | 30 | |
| ILy | D2-2.1.2 | 31 | * |
| YCS | D2-2.2.2 | 32 | # |
| IVV | D2-2.3.2 | 33 | |
| Lyy | D2-2.1.3 | 34 | * |
| CSS | D2-2.2.3 | 35 | # |
| VVV | D2-2.3.3 | 36 | |
| yyY | D2-2.1.4 | 37 | * |
| SST | D2-2.2.4 | 38 | |
| VVP | D2-2.3.4 | 39 | |
| yYQ | D2-2.1.5 | 40 | * |
| STS | D2-2.2.5 | 41 | |
| VPA | D2-2.3.5 | 42 | |
| YQL | D2-2.1.6 | 43 | |
| TSC | D2-2.2.6 | 44 | # |
| PAA | D2-2.3.6 | 45 | |
| QLL | D2-2.1.7 | 46 | |
| SCY | D2-2.2.7 | 47 | # |
| AAI | D2-2.3.7 | 48 | |
| LLY | D2-2.1.8 | 49 | |
| CYT | D2-2.2.8 | 50 | # |
| ILY | D2-8.1.2 | 51 | |
| YCT | D2-8.2.2 | 52 | # |
| IVL | D2-8.3.2 | 53 | |
| LYy | D2-8.1.3 | 54 | * |
| CTN | D2-8.2.3 | 55 | # |
| VLM | D2-8.3.3 | 56 | |
| YyW | D2-8.1.4 | 57 | * |
| TNG | D2-8.2.4 | 58 | |
| LMV | D2-8.3.4 | 59 | |
| yWC | D2-8.1.5 | 60 | *# |
| NGV | D2-8.2.5 | 61 | |
| MVY | D2-8.3.5 | 62 | |
| WCM | D2-8.1.6 | 63 | # |
| GVC | D2-8.2.6 | 64 | # |
| VYA | D2-8.3.6 | 65 | |
| CML | D2-8.1.7 | 66 | # |
| VCY | D2-8.2.7 | 67 | # |
| YAI | D2-8.3.7 | 68 | |
| MLY | D2-8.1.8 | 69 | |
| LyW | D2-15.1.3 | 70 | * |
| CSG | D2-15.2.3 | 71 | # |
| yWW | D2-15.1.4 | 72 | * |
| SGG | D2-15.2.4 | 73 | |
| WWy | D2-15.1.5 | 74 | * |
| GGS | D2-15.2.5 | 75 | |
| VVA | D2-15.3.5 | 76 | |
| WyL | D2-15.1.6 | 77 | * |
| GSC | D2-15.2.6 | 78 | # |
| VAA | D2-15.3.6 | 79 | |
| yLL | D2-15.1.7 | 80 | * |
| AAT | D2-15.3.7 | 81 | |
| LLL | D2-15.1.8 | 82 | |
| CYS | D2-15.2.8 | 83 | # |
| SIL | D2-21.1.1 | 84 | |
| AYC | D2-21.2.1 | 85 | # |
| HIV | D2-21.3.1 | 86 | |
| ILW | D2-21.1.2 | 87 | |
| YCG | D2-21.2.2 | 88 | # |
| LWW | D2-21.1.3 | 89 | |
| CGG | D2-21.2.3 | 90 | # |
| WWw | D2-21.1.4 | 91 | * |
| GGD | D2-21.2.4 | 92 | |
| VVT | D2-21.3.4 | 93 | |
| WwL | D2-21.1.5 | 94 | * |
| GDC | D2-21.2.5 | 95 | # |
| VTA | D2-21.3.5 | 96 | |
| wLL | D2-21.1.6 | 97 | * |
| DCY | D2-21.2.6 | 98 | # |
| TAI | D2-21.3.6 | 99 | |
| LLF | D2-21.1.7 | 100 | |
| VLR | D3-3.1.1 | 101 | |
| YYD | D3-3.2.1 | 102 | |
| ITI | D3-3.3.1 | 103 | |
| LRF | D3-3.1.2 | 104 | |
| YDF | D3-3.2.2 | 105 | |
| TIF | D3-3.3.2 | 106 | |
| RFL | D3-3.1.3 | 107 | |
| DFW | D3-3.2.3 | 108 | |
| IFG | D3-3.3.3 | 109 | |
| FLE | D3-3.1.4 | 110 | |
| FWS | D3-3.2.4 | 111 | |
| FGV | D3-3.3.4 | 112 | |
| LEW | D3-3.1.5 | 113 | |
| WSG | D3-3.2.5 | 114 | |
| GVV | D3-3.3.5 | 115 | |
| EWL | D3-3.1.6 | 116 | |
| SGY | D3-3.2.6 | 117 | |
| VVI | D3-3.3.6 | 118 | |
| WLL | D3-3.1.7 | 119 | |
| GYY | D3-3.2.7 | 120 | |
| VII | D3-3.3.7 | 121 | |
| YYT | D3-3.2.8 | 122 | |
| LRY | D3-9.1.2 | 123 | |
| YDI | D3-9.2.2 | 124 | |
| RYF | D3-9.1.3 | 125 | |
| DIL | D3-9.2.3 | 126 | |
| IFy | D3-9.3.3 | 127 | * |
| YFD | D3-9.1.4 | 128 | |
| ILT | D3-9.2.4 | 129 | |
| FyL | D3-9.3.4 | 130 | * |
| FDW | D3-9.1.5 | 131 | |
| LTG | D3-9.2.5 | 132 | |
| yLV | D3-9.3.5 | 133 | * |
| DWL | D3-9.1.6 | 134 | |
| TGY | D3-9.2.6 | 135 | |
| LVI | D3-9.3.6 | 136 | |
| LLy | D3-9.1.8 | 137 | * |
| YYN | D3-9.2.8 | 138 | |
| VLL | D3-10.1.1 | 139 | |
| YYY | D3-10.2.1 | 140 | |
| ITM | D3-10.3.1 | 141 | |
| LLW | D3-10.1.2 | 142 | |
| YYG | D3-10.2.2 | 143 | |
| TMV | D3-10.3.2 | 144 | |
| LWF | D3-10.1.3 | 145 | |
| YGS | D3-10.2.3 | 146 | |
| MVR | D3-10.3.3 | 147 | |
| WFG | D3-10.1.4 | 148 | |
| GSG | D3-10.2.4 | 149 | |
| VRG | D3-10.3.4 | 150 | |
| FGE | D3-10.1.5 | 151 | |
| RGV | D3-10.3.5 | 152 | |
| GEL | D3-10.1.6 | 153 | |
| GVI | D3-10.3.6 | 154 | |
| VLw | D3-16.1.1 | 155 | * |
| IMI | D3-16.3.1 | 156 | |
| LwL | D3-16.1.2 | 157 | * |
| YDY | D3-16.2.2 | 158 | |
| MIT | D3-16.3.2 | 159 | |
| wLR | D3-16.1.3 | 160 | * |
| DYV | D3-16.2.3 | 161 | |
| ITF | D3-16.3.3 | 162 | |
| LRL | D3-16.1.4 | 163 | |
| YVW | D3-16.2.4 | 164 | |
| TFG | D3-16.3.4 | 165 | |
| RLG | D3-16.1.5 | 166 | |
| VWG | D3-16.2.5 | 167 | |
| FGG | D3-16.3.5 | 168 | |
| LGE | D3-16.1.6 | 169 | |
| WGS | D3-16.2.6 | 170 | |
| GGV | D3-16.3.6 | 171 | |
| ELS | D3-16.1.8 | 172 | |
| SYR | D3-16.2.8 | 173 | |
| VIV | D3-16.3.8 | 174 | |
| LSL | D3-16.1.9 | 175 | |
| YRY | D3-16.2.9 | 176 | |
| IVI | D3-16.3.9 | 177 | |
| SLY | D3-16.1.10 | 178 | |
| RYT | D3-16.2.10 | 179 | |
| LLw | D3-22.1.2 | 180 | * |
| TMI | D3-22.3.2 | 181 | |
| Lwy | D3-22.1.3 | 182 | * |
| YDS | D3-22.2.3 | 183 | |
| MIV | D3-22.3.3 | 184 | |
| wyy | D3-22.1.4 | 185 | * |
| DSS | D3-22.2.4 | 186 | |
| yyW | D3-22.1.5 | 187 | * |
| SSG | D3-22.2.5 | 188 | |
| yWL | D3-22.1.6 | 189 | * |
| VIT | D3-22.3.7 | 190 | |
| wLQ | D4-4.1.1 | 191 | * |
| DYS | D4-4.2.1 | 192 | |
| TTV | D4-4.3.1 | 193 | |
| LQy | D4-4.1.2 | 194 | * |
| YSN | D4-4.2.2 | 195 | |
| TVT | D4-4.3.2 | 196 | |
| QyL | D4-4.1.3 | 197 | * |
| SNY | D4-4.2.3 | 198 | |
| DYG | D4-17.2.1 | 199 | |
| LRw | D4-17.1.2 | 200 | * |
| YGD | D4-17.2.2 | 201 | |
| RwL | D4-17.1.3 | 202 | * |
| GDY | D4-17.2.3 | 203 | |
| LRW | D4-23.1.2 | 204 | |
| YGG | D4-23.2.2 | 205 | |
| TVV | D4-23.3.2 | 206 | |
| RWy | D4-23.1.3 | 207 | * |
| GGN | D4-23.2.3 | 208 | |
| GNS | D4-23.2.4 | 209 | |
| VDT | D5-5.1.1 | 210 | |
| WIQ | D5-5.2.1 | 211 | |
| GYS | D5-5.3.1 | 212 | |
| DTA | D5-5.1.2 | 213 | |
| IQL | D5-5.2.2 | 214 | |
| YSY | D5-5.3.2 | 215 | |
| TAM | D5-5.1.3 | 216 | |
| QLW | D5-5.2.3 | 217 | |
| SYG | D5-5.3.3 | 218 | |
| AMV | D5-5.1.4 | 219 | |
| LWL | D5-5.2.4 | 220 | |
| YGY | D5-5.3.4 | 221 | |
| VDI | D5-12.1.1 | 222 | |
| WIy | D5-12.2.1 | 223 | * |
| IyW | D5-12.2.2 | 224 | * |
| IVA | D5-12.1.3 | 225 | |
| VAT | D5-12.1.4 | 226 | |
| WLR | D5-12.2.4 | 227 | |
| GYD | D5-12.3.4 | 228 | |
| ATI | D5-12.1.5 | 229 | |
| VEM | D5-24.1.1 | 230 | |
| yRW | D5-24.2.1 | 231 | * |
| RDG | D5-24.3.1 | 232 | |
| EMA | D5-24.1.2 | 233 | |
| RWL | D5-24.2.2 | 234 | |
| DGY | D5-24.3.2 | 235 | |
| MAT | D5-24.1.3 | 236 | |
| WLQ | D5-24.2.3 | 237 | |
| GYN | D5-24.3.3 | 238 | |
| LQL | D5-24.2.4 | 239 | |
| YNY | D5-24.3.4 | 240 | |
| EYS | D6-6.1.1 | 241 | |
| SIA | D6-6.2.1 | 242 | |
| VyQ | D6-6.3.1 | 243 | * |
| YSS | D6-6.1.2 | 244 | |
| IAA | D6-6.2.2 | 245 | |
| yQL | D6-6.3.2 | 246 | * |
| SSS | D6-6.1.3 | 247 | |
| AAR | D6-6.2.3 | 248 | |
| QLV | D6-6.3.3 | 249 | |
| GIA | D6-13.2.1 | 250 | |
| yQQ | D6-13.3.2 | 251 | * |
| AAA | D6-13.2.3 | 252 | |
| QQL | D6-13.3.3 | 253 | |
| SSW | D6-13.1.4 | 254 | |
| AAG | D6-13.2.4 | 255 | |
| SWY | D6-13.1.5 | 256 | |
| IAV | D6-19.2.2 | 257 | |
| yQW | D6-19.3.2 | 258 | * |
| AVA | D6-19.2.3 | 259 | |
| QWL | D6-19.3.3 | 260 | |
| SGW | D6-19.1.4 | 261 | |
| VAG | D6-19.2.4 | 262 | |
| WLV | D6-19.3.4 | 263 | |
| GWY | D6-19.1.5 | 264 | |
| yLG | D7-27.2.1 | 265 | * |
| NWG | D7-27.3.1 | 266 | |

TABLE 12
Distinct tetramers that can be extracted
from human D segments

| | | | |
|---|---|---|---|
| GTTG | D1-1.1.1 | (SEQ ID NO: 257) | 1 |
| VQLE | D1-1.2.1 | (SEQ ID NO: 258) | 2 |
| YNWN | D1-1.3.1 | (SEQ ID NO: 259) | 3 |
| TTGT | D1-1.1.2 | (SEQ ID NO: 263) | 4 |
| QLER | D1-1.2.2 | (SEQ ID NO: 264) | 5 |
| NWND | D1-1.3.2 | (SEQ ID NO: 265) | 6 |
| GITG | D1-7.1.1 | (SEQ ID NO: 266) | 7 |
| VyLE | D1-7.2.1 | (SEQ ID NO: 267) | 8 |
| ITGT | D1-7.1.2 | (SEQ ID NO: 271) | 9 |
| yLEL | D1-7.2.2 | (SEQ ID NO: 272) | 10 |
| NWNY | D1-7.3.2 | (SEQ ID NO: 273) | 11 |
| yLER | D1-20.2.2 | (SEQ ID NO: 275) | 12 |
| GIVG | D1-26.1.1 | (SEQ ID NO: 276) | 13 |
| VyWE | D1-26.2.1 | (SEQ ID NO: 277) | 14 |
| YSGS | D1-26.3.1 | (SEQ ID NO: 278) | 15 |
| IVGA | D1-26.1.2 | (SEQ ID NO: 285) | 16 |
| yWEL | D1-26.2.2 | (SEQ ID NO: 286) | 17 |
| SGSY | D1-26.3.2 | (SEQ ID NO: 287) | 18 |
| VGAT | D1-26.1.3 | (SEQ ID NO: 291) | 19 |
| WELL | D1-26.2.3 | (SEQ ID NO: 292) | 20 |
| GSYY | D1-26.3.3 | (SEQ ID NO: 293) | 21 |
| RILy | D2-2.1.1 | (SEQ ID NO: 294) | 22 |
| GYCS | D2-2.2.1 | (SEQ ID NO: 295) | 23 |
| DIVV | D2-2.3.1 | (SEQ ID NO: 296) | 24 |
| ILyy | D2-2.1.2 | (SEQ ID NO: 303) | 25 |
| YCSS | D2-2.2.2 | (SEQ ID NO: 304) | 26 |
| IVVV | D2-2.3.2 | (SEQ ID NO: 305) | 27 |
| LyyY | D2-2.1.3 | (SEQ ID NO: 312) | 28 |
| CSST | D2-2.2.3 | (SEQ ID NO: 313) | 29 |
| VVVP | D2-2.3.3 | (SEQ ID NO: 314) | 30 |
| yyYQ | D2-2.1.4 | (SEQ ID NO: 321) | 31 |
| SSTS | D2-2.2.4 | (SEQ ID NO: 322) | 32 |
| VVPA | D2-2.3.4 | (SEQ ID NO: 323) | 33 |
| yYQL | D2-2.1.5 | (SEQ ID NO: 330) | 34 |
| STSC | D2-2.2.5 | (SEQ ID NO: 331) | 35 |
| VPAA | D2-2.3.5 | (SEQ ID NO: 332) | 36 |
| YQLL | D2-2.1.6 | (SEQ ID NO: 338) | 37 |
| TSCY | D2-2.2.6 | (SEQ ID NO: 339) | 38 |
| PAAI | D2-2.3.6 | (SEQ ID NO: 340) | 39 |
| QLLY | D2-2.1.7 | (SEQ ID NO: 343) | 40 |
| SCYT | D2-2.2.7 | (SEQ ID NO: 344) | 41 |
| RILY | D2-8.1.1 | (SEQ ID NO: 345) | 42 |
| GYCT | D2-8.2.1 | (SEQ ID NO: 346) | 43 |
| DIVL | D2-8.3.1 | (SEQ ID NO: 347) | 44 |
| ILYy | D2-8.1.2 | (SEQ ID NO: 354) | 45 |
| YCTN | D2-8.2.2 | (SEQ ID NO: 355) | 46 |
| IVLM | D2-8.3.2 | (SEQ ID NO: 356) | 47 |
| LYyW | D2-8.1.3 | (SEQ ID NO: 363) | 48 |
| CTNG | D2-8.2.3 | (SEQ ID NO: 364) | 49 |
| VLMV | D2-8.3.3 | (SEQ ID NO: 365) | 50 |
| YyWC | D2-8.1.4 | (SEQ ID NO: 372) | 51 |
| TNGV | D2-8.2.4 | (SEQ ID NO: 373) | 52 |
| LMVY | D2-8.3.4 | (SEQ ID NO: 374) | 53 |
| yWCM | D2-8.1.5 | (SEQ ID NO: 381) | 54 |
| NGVC | D2-8.2.5 | (SEQ ID NO: 382) | 55 |
| MVYA | D2-8.3.5 | (SEQ ID NO: 383) | 56 |
| WCML | D2-8.1.6 | (SEQ ID NO: 389) | 57 |
| GVCY | D2-8.2.6 | (SEQ ID NO: 390) | 58 |
| VYAI | D2-8.3.6 | (SEQ ID NO: 391) | 59 |
| CMLY | D2-8.1.7 | (SEQ ID NO: 394) | 60 |
| VCYT | D2-8.2.7 | (SEQ ID NO: 395) | 61 |
| ILyW | D2-15.1.2 | (SEQ ID NO: 401) | 62 |
| YCSG | D2-15.2.2 | (SEQ ID NO: 402) | 63 |
| LyWW | D2-15.1.3 | (SEQ ID NO: 409) | 64 |
| CSGG | D2-15.2.3 | (SEQ ID NO: 410) | 65 |
| VVVV | D2-15.3.3 | (SEQ ID NO: 411) | 66 |
| yWWy | D2-15.1.4 | (SEQ ID NO: 418) | 67 |
| SGGS | D2-15.2.4 | (SEQ ID NO: 419) | 68 |
| VVVA | D2-15.3.4 | (SEQ ID NO: 420) | 69 |
| WWyL | D2-15.1.5 | (SEQ ID NO: 427) | 70 |
| GGSC | D2-15.2.5 | (SEQ ID NO: 428) | 71 |
| VVAA | D2-15.3.5 | (SEQ ID NO: 429) | 72 |
| WyLL | D2-15.1.6 | (SEQ ID NO: 435) | 73 |
| GSCY | D2-15.2.6 | (SEQ ID NO: 436) | 74 |
| VAAT | D2-15.3.6 | (SEQ ID NO: 437) | 75 |
| yLLL | D2-15.1.7 | (SEQ ID NO: 440) | 76 |
| SCYS | D2-15.2.7 | (SEQ ID NO: 441) | 77 |
| SILW | D2-21.1.1 | (SEQ ID NO: 442) | 78 |
| AYCG | D2-21.2.1 | (SEQ ID NO: 443) | 79 |
| HIVV | D2-21.3.1 | (SEQ ID NO: 444) | 80 |
| ILWW | D2-21.1.2 | (SEQ ID NO: 451) | 81 |
| YCGG | D2-21.2.2 | (SEQ ID NO: 452) | 82 |
| LWWw | D2-21.1.3 | (SEQ ID NO: 459) | 83 |
| CGGD | D2-21.2.3 | (SEQ ID NO: 460) | 84 |
| VVVT | D2-21.3.3 | (SEQ ID NO: 461) | 85 |
| WWwL | D2-21.1.4 | (SEQ ID NO: 468) | 86 |
| GGDC | D2-21.2.4 | (SEQ ID NO: 469) | 87 |
| VVTA | D2-21.3.4 | (SEQ ID NO: 470) | 88 |
| WwLL | D2-21.1.5 | (SEQ ID NO: 476) | 89 |
| GDCY | D2-21.2.5 | (SEQ ID NO: 477) | 90 |
| VTAI | D2-21.3.5 | (SEQ ID NO: 478) | 91 |
| wLLF | D2-21.1.6 | (SEQ ID NO: 481) | 92 |
| DCYS | D2-21.2.6 | (SEQ ID NO: 482) | 93 |
| VLRF | D3-3.1.1 | (SEQ ID NO: 483) | 94 |
| YYDF | D3-3.2.1 | (SEQ ID NO: 484) | 95 |
| ITIF | D3-3.3.1 | (SEQ ID NO: 485) | 96 |
| LRFL | D3-3.1.2 | (SEQ ID NO: 492) | 97 |
| YDFW | D3-3.2.2 | (SEQ ID NO: 493) | 98 |
| TIFG | D3-3.3.2 | (SEQ ID NO: 494) | 99 |
| RFLE | D3-3.1.3 | (SEQ ID NO: 501) | 100 |
| DFWS | D3-3.2.3 | (SEQ ID NO: 502) | 101 |
| IFGV | D3-3.3.3 | (SEQ ID NO: 503) | 102 |
| FLEW | D3-3.1.4 | (SEQ ID NO: 510) | 103 |
| FWSG | D3-3.2.4 | (SEQ ID NO: 511) | 104 |
| FGVV | D3-3.3.4 | (SEQ ID NO: 512) | 105 |
| LEWL | D3-3.1.5 | (SEQ ID NO: 519) | 106 |
| WSGY | D3-3.2.5 | (SEQ ID NO: 520) | 107 |
| GVVI | D3-3.3.5 | (SEQ ID NO: 521) | 108 |
| EWLL | D3-3.1.6 | (SEQ ID NO: 527) | 109 |
| SGYY | D3-3.2.6 | (SEQ ID NO: 528) | 110 |
| VVII | D3-3.3.6 | (SEQ ID NO: 529) | 111 |
| WLLY | D3-3.1.7 | (SEQ ID NO: 532) | 112 |
| GYYT | D3-3.2.7 | (SEQ ID NO: 533) | 113 |
| VLRY | D3-9.1.1 | (SEQ ID NO: 534) | 114 |
| YYDI | D3-9.2.1 | (SEQ ID NO: 535) | 115 |
| LRYF | D3-9.1.2 | (SEQ ID NO: 542) | 116 |
| YDIL | D3-9.2.2 | (SEQ ID NO: 543) | 117 |
| TIFy | D3-9.3.2 | (SEQ ID NO: 544) | 118 |
| RYFD | D3-9.1.3 | (SEQ ID NO: 551) | 119 |
| DILT | D3-9.2.3 | (SEQ ID NO: 552) | 120 |
| IFyL | D3-9.3.3 | (SEQ ID NO: 553) | 121 |
| YFDW | D3-9.1.4 | (SEQ ID NO: 560) | 122 |
| ILTG | D3-9.2.4 | (SEQ ID NO: 561) | 123 |
| FyLV | D3-9.3.4 | (SEQ ID NO: 562) | 124 |
| FDWL | D3-9.1.5 | (SEQ ID NO: 569) | 125 |
| LTGY | D3-9.2.5 | (SEQ ID NO: 570) | 126 |
| yLVI | D3-9.3.5 | (SEQ ID NO: 571) | 127 |
| DWLL | D3-9.1.6 | (SEQ ID NO: 577) | 128 |
| TGYY | D3-9.2.6 | (SEQ ID NO: 578) | 129 |
| LVII | D3-9.3.6 | (SEQ ID NO: 579) | 130 |
| WLLy | D3-9.1.7 | (SEQ ID NO: 582) | 131 |
| GYYN | D3-9.2.7 | (SEQ ID NO: 583) | 132 |
| VLLW | D3-10.1.1 | (SEQ ID NO: 584) | 133 |
| YYYG | D3-10.2.1 | (SEQ ID NO: 585) | 134 |
| ITMV | D3-10.3.1 | (SEQ ID NO: 586) | 135 |
| LLWF | D3-10.1.2 | (SEQ ID NO: 593) | 136 |
| YYGS | D3-10.2.2 | (SEQ ID NO: 594) | 137 |
| TMVR | D3-10.3.2 | (SEQ ID NO: 595) | 138 |
| LWFG | D3-10.1.3 | (SEQ ID NO: 602) | 139 |
| YGSG | D3-10.2.3 | (SEQ ID NO: 603) | 140 |
| MVRG | D3-10.3.3 | (SEQ ID NO: 604) | 141 |
| WFGE | D3-10.1.4 | (SEQ ID NO: 611) | 142 |
| GSGS | D3-10.2.4 | (SEQ ID NO: 612) | 143 |
| VRGV | D3-10.3.4 | (SEQ ID NO: 613) | 144 |
| FGEL | D3-10.1.5 | (SEQ ID NO: 620) | 145 |
| RGVI | D3-10.3.5 | (SEQ ID NO: 621) | 146 |
| GELL | D3-10.1.6 | (SEQ ID NO: 626) | 147 |
| GVII | D3-10.3.6 | (SEQ ID NO: 627) | 148 |
| ELLy | D3-10.1.7 | (SEQ ID NO: 630) | 149 |
| SYYN | D3-10.2.7 | (SEQ ID NO: 631) | 150 |
| VLwL | D3-16.1.1 | (SEQ ID NO: 632) | 151 |
| YYDY | D3-16.2.1 | (SEQ ID NO: 633) | 152 |
| IMIT | D3-16.3.1 | (SEQ ID NO: 634) | 153 |
| LwLR | D3-16.1.2 | (SEQ ID NO: 641) | 154 |
| YDYV | D3-16.2.2 | (SEQ ID NO: 642) | 155 |
| MITF | D3-16.3.2 | (SEQ ID NO: 643) | 156 |
| wLRL | D3-16.1.3 | (SEQ ID NO: 650) | 157 |
| DYVW | D3-16.2.3 | (SEQ ID NO: 651) | 158 |
| ITFG | D3-16.3.3 | (SEQ ID NO: 652) | 159 |
| LRLG | D3-16.1.4 | (SEQ ID NO: 659) | 160 |
| YVWG | D3-16.2.4 | (SEQ ID NO: 660) | 161 |
| TFGG | D3-16.3.4 | (SEQ ID NO: 661) | 162 |
| RLGE | D3-16.1.5 | (SEQ ID NO: 668) | 163 |
| VWGS | D3-16.2.5 | (SEQ ID NO: 669) | 164 |
| FGGV | D3-16.3.5 | (SEQ ID NO: 670) | 165 |
| LGEL | D3-16.1.6 | (SEQ ID NO: 677) | 166 |
| WGSY | D3-16.2.6 | (SEQ ID NO: 678) | 167 |
| GGVI | D3-16.3.6 | (SEQ ID NO: 679) | 168 |
| GELS | D3-16.1.7 | (SEQ ID NO: 686) | 169 |
| GSYR | D3-16.2.7 | (SEQ ID NO: 687) | 170 |
| GVIV | D3-16.3.7 | (SEQ ID NO: 688) | 171 |
| ELSL | D3-16.1.8 | (SEQ ID NO: 694) | 172 |
| SYRY | D3-16.2.8 | (SEQ ID NO: 695) | 173 |
| VIVI | D3-16.3.8 | (SEQ ID NO: 696) | 174 |
| LSLY | D3-16.1.9 | (SEQ ID NO: 699) | 175 |
| YRYT | D3-16.2.9 | (SEQ ID NO: 700) | 176 |
| VLLw | D3-22.1.1 | (SEQ ID NO: 701) | 177 |
| YYYD | D3-22.2.1 | (SEQ ID NO: 702) | 178 |
| ITMI | D3-22.3.1 | (SEQ ID NO: 703) | 179 |
| LLwy | D3-22.1.2 | (SEQ ID NO: 710) | 180 |
| YYDS | D3-22.2.2 | (SEQ ID NO: 711) | 181 |
| TMIV | D3-22.3.2 | (SEQ ID NO: 712) | 182 |
| Lwyy | D3-22.1.3 | (SEQ ID NO: 719) | 183 |
| YDSS | D3-22.2.3 | (SEQ ID NO: 720) | 184 |
| MIVV | D3-22.3.3 | (SEQ ID NO: 721) | 185 |
| wyyW | D3-22.1.4 | (SEQ ID NO: 728) | 186 |
| DSSG | D3-22.2.4 | (SEQ ID NO: 729) | 187 |
| yyWL | D3-22.1.5 | (SEQ ID NO: 736) | 188 |
| SSGY | D3-22.2.5 | (SEQ ID NO: 737) | 189 |
| VVVI | D3-22.3.5 | (SEQ ID NO: 738) | 190 |
| yWLL | D3-22.1.6 | (SEQ ID NO: 744) | 191 |
| VVIT | D3-22.3.6 | (SEQ ID NO: 745) | 192 |
| WLLL | D3-22.1.7 | (SEQ ID NO: 748) | 193 |
| GYYY | D3-22.2.7 | (SEQ ID NO: 749) | 194 |
| wLQy | D4-4.1.1 | (SEQ ID NO: 750) | 195 |
| DYSN | D4-4.2.1 | (SEQ ID NO: 751) | 196 |
| TTVT | D4-4.3.1 | (SEQ ID NO: 752) | 197 |
| LQyL | D4-4.1.2 | (SEQ ID NO: 755) | 198 |
| YSNY | D4-4.2.2 | (SEQ ID NO: 756) | 199 |
| wLRw | D4-17.1.1 | (SEQ ID NO: 757) | 200 |
| DYGD | D4-17.2.1 | (SEQ ID NO: 758) | 201 |
| LRwL | D4-17.1.2 | (SEQ ID NO: 761) | 202 |
| YGDY | D4-17.2.2 | (SEQ ID NO: 762) | 203 |
| wLRW | D4-23.1.1 | (SEQ ID NO: 763) | 204 |
| DYGG | D4-23.2.1 | (SEQ ID NO: 764) | 205 |
| TTVV | D4-23.3.1 | (SEQ ID NO: 765) | 206 |
| LRWy | D4-23.1.2 | (SEQ ID NO: 771) | 207 |
| YGGN | D4-23.2.2 | (SEQ ID NO: 772) | 208 |
| TVVT | D4-23.3.2 | (SEQ ID NO: 773) | 209 |
| RWyL | D4-23.1.3 | (SEQ ID NO: 776) | 210 |
| GGNS | D4-23.2.3 | (SEQ ID NO: 777) | 211 |
| VDTA | D5-5.1.1 | (SEQ ID NO: 778) | 212 |
| WIQL | D5-5.2.1 | (SEQ ID NO: 779) | 213 |
| GYSY | D5-5.3.1 | (SEQ ID NO: 780) | 214 |
| DTAM | D5-5.1.2 | (SEQ ID NO: 787) | 215 |
| IQLW | D5-5.2.2 | (SEQ ID NO: 788) | 216 |
| YSYG | D5-5.3.2 | (SEQ ID NO: 789) | 217 |
| TAMV | D5-5.1.3 | (SEQ ID NO: 793) | 218 |
| QLWL | D5-5.2.3 | (SEQ ID NO: 794) | 219 |
| SYGY | D5-5.3.3 | (SEQ ID NO: 795) | 220 |
| VDIV | D5-12.1.1 | (SEQ ID NO: 796) | 221 |
| WIyW | D5-12.2.1 | (SEQ ID NO: 797) | 222 |
| GYSG | D5-12.3.1 | (SEQ ID NO: 798) | 223 |
| DIVA | D5-12.1.2 | (SEQ ID NO: 805) | 224 |
| IyWL | D5-12.2.2 | (SEQ ID NO: 806) | 225 |
| YSGY | D5-12.3.2 | (SEQ ID NO: 807) | 226 |
| IVAT | D5-12.1.3 | (SEQ ID NO: 814) | 227 |
| yWLR | D5-12.2.3 | (SEQ ID NO: 815) | 228 |
| SGYD | D5-12.3.3 | (SEQ ID NO: 816) | 229 |
| VATI | D5-12.1.4 | (SEQ ID NO: 820) | 230 |
| WLRL | D5-12.2.4 | (SEQ ID NO: 821) | 231 |
| GYDY | D5-12.3.4 | (SEQ ID NO: 822) | 232 |
| VEMA | D5-24.1.1 | (SEQ ID NO: 823) | 233 |
| yRWL | D5-24.2.1 | (SEQ ID NO: 824) | 234 |
| RDGY | D5-24.3.1 | (SEQ ID NO: 825) | 235 |
| EMAT | D5-24.1.2 | (SEQ ID NO: 832) | 236 |
| RWLQ | D5-24.2.2 | (SEQ ID NO: 833) | 237 |
| DGYN | D5-24.3.2 | (SEQ ID NO: 834) | 238 |
| MATI | D5-24.1.3 | (SEQ ID NO: 838) | 239 |
| WLQL | D5-24.2.3 | (SEQ ID NO: 839) | 240 |
| GYNY | D5-24.3.3 | (SEQ ID NO: 840) | 241 |
| EYSS | D6-6.1.1 | (SEQ ID NO: 841) | 242 |
| SIAA | D6-6.2.1 | (SEQ ID NO: 842) | 243 |
| VyQL | D6-6.3.1 | (SEQ ID NO: 843) | 244 |
| YSSS | D6-6.1.2 | (SEQ ID NO: 848) | 245 |
| IAAR | D6-6.2.2 | (SEQ ID NO: 849) | 246 |
| yQLV | D6-6.3.2 | (SEQ ID NO: 850) | 247 |
| SSSS | D6-6.1.3 | (SEQ ID NO: 852) | 248 |
| GYSS | D6-13.1.1 | (SEQ ID NO: 853) | 249 |
| GIAA | D6-13.2.1 | (SEQ ID NO: 854) | 250 |
| VyQQ | D6-13.3.1 | (SEQ ID NO: 855) | 251 |
| IAAA | D6-13.2.2 | (SEQ ID NO: 862) | 252 |
| yQQL | D6-13.3.2 | (SEQ ID NO: 863) | 253 |
| SSSW | D6-13.1.3 | (SEQ ID NO: 868) | 254 |
| AAAG | D6-13.2.3 | (SEQ ID NO: 869) | 255 |
| QQLV | D6-13.3.3 | (SEQ ID NO: 870) | 256 |
| SSWY | D6-13.1.4 | (SEQ ID NO: 872) | 257 |
| GIAV | D6-19.2.1 | (SEQ ID NO: 873) | 258 |
| VyQW | D6-19.3.1 | (SEQ ID NO: 874) | 259 |
| YSSG | D6-19.1.2 | (SEQ ID NO: 881) | 260 |
| IAVA | D6-19.2.2 | (SEQ ID NO: 882) | 261 |
| yQWL | D6-19.3.2 | (SEQ ID NO: 883) | 262 |
| SSGW | D6-19.1.3 | (SEQ ID NO: 888) | 263 |
| AVAG | D6-19.2.3 | (SEQ ID NO: 889) | 264 |
| QWLV | D6-19.3.3 | (SEQ ID NO: 890) | 265 |
| SGWY | D6-19.1.4 | (SEQ ID NO: 892) | 266 |

TABLE 13
Pentamers that can be extracted from human
D segments

| | | | |
|---|---|---|---|
| GTTGT | D1-1.1.1 | (SEQ ID NO: 260) | 1 |
| VQLER | D1-1.2.1 | (SEQ ID NO: 261) | 2 |
| YNWND | D1-1.3.1 | (SEQ ID NO: 262) | 3 |
| GITGT | D1-7.1.1 | (SEQ ID NO: 268) | 4 |
| VyLEL | D1-7.2.1 | (SEQ ID NO: 269) | 5 |
| YNWNY | D1-7.3.1 | (SEQ ID NO: 270) | 6 |
| VyLER | D1-20.2.1 | (SEQ ID NO: 274) | 7 |
| GIVGA | D1-26.1.1 | (SEQ ID NO: 279) | 8 |
| VyWEL | D1-26.2.1 | (SEQ ID NO: 280) | 9 |
| YSGSY | D1-26.3.1 | (SEQ ID NO: 281) | 10 |
| IVGAT | D1-26.1.2 | (SEQ ID NO: 288) | 11 |
| yWELL | D1-26.2.2 | (SEQ ID NO: 289) | 12 |
| SGSYY | D1-26.3.2 | (SEQ ID NO: 290) | 13 |
| RILyy | D2-2.1.1 | (SEQ ID NO: 297) | 14 |
| GYCSS | D2-2.2.1 | (SEQ ID NO: 298) | 15 |
| DIVVV | D2-2.3.1 | (SEQ ID NO: 299) | 16 |
| ILyyY | D2-2.1.2 | (SEQ ID NO: 306) | 17 |
| YCSST | D2-2.2.2 | (SEQ ID NO: 307) | 18 |
| IVVVP | D2-2.3.2 | (SEQ ID NO: 308) | 19 |
| LyyYQ | D2-2.1.3 | (SEQ ID NO: 315) | 20 |
| CSSTS | D2-2.2.3 | (SEQ ID NO: 316) | 21 |
| VVVPA | D2-2.3.3 | (SEQ ID NO: 317) | 22 |
| yyYQL | D2-2.1.4 | (SEQ ID NO: 324) | 23 |
| SSTSC | D2-2.2.4 | (SEQ ID NO: 325) | 24 |
| VVPAA | D2-2.3.4 | (SEQ ID NO: 326) | 25 |
| yYQLL | D2-2.1.5 | (SEQ ID NO: 333) | 26 |
| STSCY | D2-2.2.5 | (SEQ ID NO: 334) | 27 |
| VPAAI | D2-2.3.5 | (SEQ ID NO: 335) | 28 |
| YQLLY | D2-2.1.6 | (SEQ ID NO: 341) | 29 |
| TSCYT | D2-2.2.6 | (SEQ ID NO: 342) | 30 |
| RILYy | D2-8.1.1 | (SEQ ID NO: 348) | 31 |
| GYCTN | D2-8.2.1 | (SEQ ID NO: 349) | 32 |
| DIVLM | D2-8.3.1 | (SEQ ID NO: 350) | 33 |
| ILYyW | D2-8.1.2 | (SEQ ID NO: 357) | 34 |
| YCTNG | D2-8.2.2 | (SEQ ID NO: 358) | 35 |
| IVLMV | D2-8.3.2 | (SEQ ID NO: 359) | 36 |
| LYyWC | D2-8.1.3 | (SEQ ID NO: 366) | 37 |
| CTNGV | D2-8.2.3 | (SEQ ID NO: 367) | 38 |
| VLMVY | D2-8.3.3 | (SEQ ID NO: 368) | 39 |
| YyWCM | D2-8.1.4 | (SEQ ID NO: 375) | 40 |
| TNGVC | D2-8.2.4 | (SEQ ID NO: 376) | 41 |
| LMVYA | D2-8.3.4 | (SEQ ID NO: 377) | 42 |
| yWCML | D2-8.1.5 | (SEQ ID NO: 384) | 43 |
| NGVCY | D2-8.2.5 | (SEQ ID NO: 385) | 44 |
| MVYAI | D2-8.3.5 | (SEQ ID NO: 386) | 45 |
| WCMLY | D2-8.1.6 | (SEQ ID NO: 392) | 46 |
| GVCYT | D2-8.2.6 | (SEQ ID NO: 393) | 47 |
| RILyW | D2-15.1.1 | (SEQ ID NO: 396) | 48 |
| GYCSG | D2-15.2.1 | (SEQ ID NO: 397) | 49 |
| ILyWW | D2-15.1.2 | (SEQ ID NO: 403) | 50 |
| YCSGG | D2-15.2.2 | (SEQ ID NO: 404) | 51 |
| IVVVV | D2-15.3.2 | (SEQ ID NO: 405) | 52 |
| LyWWy | D2-15.1.3 | (SEQ ID NO: 412) | 53 |
| CSGGS | D2-15.2.3 | (SEQ ID NO: 413) | 54 |
| VVVVA | D2-15.3.3 | (SEQ ID NO: 414) | 55 |
| yWWyL | D2-15.1.4 | (SEQ ID NO: 421) | 56 |
| SGGSC | D2-15.2.4 | (SEQ ID NO: 422) | 57 |
| VVVAA | D2-15.3.4 | (SEQ ID NO: 423) | 58 |
| WWyLL | D2-15.1.5 | (SEQ ID NO: 430) | 59 |
| GGSCY | D2-15.2.5 | (SEQ ID NO: 431) | 60 |
| VVAAT | D2-15.3.5 | (SEQ ID NO: 432) | 61 |
| WyLLL | D2-15.1.6 | (SEQ ID NO: 438) | 62 |
| GSCYS | D2-15.2.6 | (SEQ ID NO: 439) | 63 |
| SILWW | D2-21.1.1 | (SEQ ID NO: 445) | 64 |
| AYCGG | D2-21.2.1 | (SEQ ID NO: 446) | 65 |
| HIVVV | D2-21.3.1 | (SEQ ID NO: 447) | 66 |
| ILWWw | D2-21.1.2 | (SEQ ID NO: 453) | 67 |
| YCGGD | D2-21.2.2 | (SEQ ID NO: 454) | 68 |
| IVVVT | D2-21.3.2 | (SEQ ID NO: 455) | 69 |
| LWWwL | D2-21.1.3 | (SEQ ID NO: 462) | 70 |
| CGGDC | D2-21.2.3 | (SEQ ID NO: 463) | 71 |
| VVVTA | D2-21.3.3 | (SEQ ID NO: 464) | 72 |
| WWwLL | D2-21.1.4 | (SEQ ID NO: 471) | 73 |
| GGDCY | D2-21.2.4 | (SEQ ID NO: 472) | 74 |
| VVTAI | D2-21.3.4 | (SEQ ID NO: 473) | 75 |
| WwLLF | D2-21.1.5 | (SEQ ID NO: 479) | 76 |
| GDCYS | D2-21.2.5 | (SEQ ID NO: 480) | 77 |
| VLRFL | D3-3.1.1 | (SEQ ID NO: 486) | 78 |
| YYDFW | D3-3.2.1 | (SEQ ID NO: 487) | 79 |
| ITIFG | D3-3.3.1 | (SEQ ID NO: 488) | 80 |
| LRFLE | D3-3.1.2 | (SEQ ID NO: 495) | 81 |
| YDFWS | D3-3.2.2 | (SEQ ID NO: 496) | 82 |
| TIFGV | D3-3.3.2 | (SEQ ID NO: 497) | 83 |
| RFLEW | D3-3.1.3 | (SEQ ID NO: 504) | 84 |
| DFWSG | D3-3.2.3 | (SEQ ID NO: 505) | 85 |
| IFGVV | D3-3.3.3 | (SEQ ID NO: 506) | 86 |
| FLEWL | D3-3.1.4 | (SEQ ID NO: 513) | 87 |
| FWSGY | D3-3.2.4 | (SEQ ID NO: 514) | 88 |
| FGVVI | D3-3.3.4 | (SEQ ID NO: 515) | 89 |
| LEWLL | D3-3.1.5 | (SEQ ID NO: 522) | 90 |
| WSGYY | D3-3.2.5 | (SEQ ID NO: 523) | 91 |
| GVVII | D3-3.3.5 | (SEQ ID NO: 524) | 92 |
| EWLLY | D3-3.1.6 | (SEQ ID NO: 530) | 93 |
| SGYYT | D3-3.2.6 | (SEQ ID NO: 531) | 94 |
| VLRYF | D3-9.1.1 | (SEQ ID NO: 536) | 95 |
| YYDIL | D3-9.2.1 | (SEQ ID NO: 537) | 96 |
| ITIFy | D3-9.3.1 | (SEQ ID NO: 538) | 97 |
| LRYFD | D3-9.1.2 | (SEQ ID NO: 545) | 98 |
| YDILT | D3-9.2.2 | (SEQ ID NO: 546) | 99 |
| TIFyL | D3-9.3.2 | (SEQ ID NO: 547) | 100 |
| RYFDW | D3-9.1.3 | (SEQ ID NO: 554) | 101 |
| DILTG | D3-9.2.3 | (SEQ ID NO: 555) | 102 |
| IFyLV | D3-9.3.3 | (SEQ ID NO: 556) | 103 |
| YFDWL | D3-9.1.4 | (SEQ ID NO: 563) | 104 |
| ILTGY | D3-9.2.4 | (SEQ ID NO: 564) | 105 |
| FyLVI | D3-9.3.4 | (SEQ ID NO: 565) | 106 |
| FDWLL | D3-9.1.5 | (SEQ ID NO: 572) | 107 |
| LTGYY | D3-9.2.5 | (SEQ ID NO: 573) | 108 |
| yLVII | D3-9.3.5 | (SEQ ID NO: 574) | 109 |
| DWLLy | D3-9.1.6 | (SEQ ID NO: 580) | 110 |
| TGYYN | D3-9.2.6 | (SEQ ID NO: 581) | 111 |
| VLLWF | D3-10.1.1 | (SEQ ID NO: 587) | 112 |
| YYYGS | D3-10.2.1 | (SEQ ID NO: 588) | 113 |
| ITMVR | D3-10.3.1 | (SEQ ID NO: 589) | 114 |
| LLWFG | D3-10.1.2 | (SEQ ID NO: 596) | 115 |
| YYGSG | D3-10.2.2 | (SEQ ID NO: 597) | 116 |
| TMVRG | D3-10.3.2 | (SEQ ID NO: 598) | 117 |
| LWFGE | D3-10.1.3 | (SEQ ID NO: 605) | 118 |
| YGSGS | D3-10.2.3 | (SEQ ID NO: 606) | 119 |
| MVRGV | D3-10.3.3 | (SEQ ID NO: 607) | 120 |
| WFGEL | D3-10.1.4 | (SEQ ID NO: 614) | 121 |
| GSGSY | D3-10.2.4 | (SEQ ID NO: 615) | 122 |
| VRGVI | D3-10.3.4 | (SEQ ID NO: 616) | 123 |
| FGELL | D3-10.1.5 | (SEQ ID NO: 622) | 124 |
| RGVII | D3-10.3.5 | (SEQ ID NO: 623) | 125 |
| GELLy | D3-10.1.6 | (SEQ ID NO: 628) | 126 |
| GSYYN | D3-10.2.6 | (SEQ ID NO: 629) | 127 |
| VLwLR | D3-16.1.1 | (SEQ ID NO: 635) | 128 |
| YYDYV | D3-16.2.1 | (SEQ ID NO: 636) | 129 |
| IMITF | D3-16.3.1 | (SEQ ID NO: 637) | 130 |
| LwLRL | D3-16.1.2 | (SEQ ID NO: 644) | 131 |
| YDYVW | D3-16.2.2 | (SEQ ID NO: 645) | 132 |
| MITFG | D3-16.3.2 | (SEQ ID NO: 646) | 133 |
| wLRLG | D3-16.1.3 | (SEQ ID NO: 653) | 134 |
| DYVWG | D3-16.2.3 | (SEQ ID NO: 654) | 135 |
| ITFGG | D3-16.3.3 | (SEQ ID NO: 655) | 136 |
| LRLGE | D3-16.1.4 | (SEQ ID NO: 662) | 137 |
| YVWGS | D3-16.2.4 | (SEQ ID NO: 663) | 138 |
| TFGGV | D3-16.3.4 | (SEQ ID NO: 664) | 139 |
| RLGEL | D3-16.1.5 | (SEQ ID NO: 671) | 140 |
| VWGSY | D3-16.2.5 | (SEQ ID NO: 672) | 141 |
| FGGVI | D3-16.3.5 | (SEQ ID NO: 673) | 142 |
| LGELS | D3-16.1.6 | (SEQ ID NO: 680) | 143 |
| WGSYR | D3-16.2.6 | (SEQ ID NO: 681) | 144 |
| GGVIV | D3-16.3.6 | (SEQ ID NO: 682) | 145 |
| GELSL | D3-16.1.7 | (SEQ ID NO: 689) | 146 |
| GSYRY | D3-16.2.7 | (SEQ ID NO: 690) | 147 |
| GVIVI | D3-16.3.7 | (SEQ ID NO: 691) | 148 |
| ELSLY | D3-16.1.8 | (SEQ ID NO: 697) | 149 |
| SYRYT | D3-16.2.8 | (SEQ ID NO: 698) | 150 |
| VLLwy | D3-22.1.1 | (SEQ ID NO: 704) | 151 |
| YYYDS | D3-22.2.1 | (SEQ ID NO: 705) | 152 |
| ITMIV | D3-22.3.1 | (SEQ ID NO: 706) | 153 |
| LLwyy | D3-22.1.2 | (SEQ ID NO: 713) | 154 |
| YYDSS | D3-22.2.2 | (SEQ ID NO: 714) | 155 |
| TMIVV | D3-22.3.2 | (SEQ ID NO: 715) | 156 |
| LwyyW | D3-22.1.3 | (SEQ ID NO: 722) | 157 |
| YDSSG | D3-22.2.3 | (SEQ ID NO: 723) | 158 |
| MIVVV | D3-22.3.3 | (SEQ ID NO: 724) | 159 |
| wyyWL | D3-22.1.4 | (SEQ ID NO: 730) | 160 |
| DSSGY | D3-22.2.4 | (SEQ ID NO: 731) | 161 |
| IVVVI | D3-22.3.4 | (SEQ ID NO: 732) | 162 |
| yyWLL | D3-22.1.5 | (SEQ ID NO: 739) | 163 |
| SSGYY | D3-22.2.5 | (SEQ ID NO: 740) | 164 |
| VVVIT | D3-22.3.5 | (SEQ ID NO: 741) | 165 |
| yWLLL | D3-22.1.6 | (SEQ ID NO: 746) | 166 |
| SGYYY | D3-22.2.6 | (SEQ ID NO: 747) | 167 |
| wLQyL | D4-4.1.1 | (SEQ ID NO: 753) | 168 |
| DYSNY | D4-4.2.1 | (SEQ ID NO: 754) | 169 |
| wLRwL | D4-17.1.1 | (SEQ ID NO: 759) | 170 |
| DYGDY | D4-17.2.1 | (SEQ ID NO: 760) | 171 |
| wLRWy | D4-23.1.1 | (SEQ ID NO: 766) | 172 |
| DYGGN | D4-23.2.1 | (SEQ ID NO: 767) | 173 |
| TTVVT | D4-23.3.1 | (SEQ ID NO: 768) | 174 |
| LRWyL | D4-23.1.2 | (SEQ ID NO: 774) | 175 |
| YGGNS | D4-23.2.2 | (SEQ ID NO: 775) | 176 |
| VDTAM | D5-5.1.1 | (SEQ ID NO: 781) | 177 |
| WIQLW | D5-5.2.1 | (SEQ ID NO: 782) | 178 |
| GYSYG | D5-5.3.1 | (SEQ ID NO: 783) | 179 |
| DTAMV | D5-5.1.2 | (SEQ ID NO: 790) | 180 |
| IQLWL | D5-5.2.2 | (SEQ ID NO: 791) | 181 |
| YSYGY | D5-5.3.2 | (SEQ ID NO: 792) | 182 |
| VDIVA | D5-12.1.1 | (SEQ ID NO: 799) | 183 |
| WIyWL | D5-12.2.1 | (SEQ ID NO: 800) | 184 |
| GYSGY | D5-12.3.1 | (SEQ ID NO: 801) | 185 |
| DIVAT | D5-12.1.2 | (SEQ ID NO: 808) | 186 |
| IyWLR | D5-12.2.2 | (SEQ ID NO: 809) | 187 |
| YSGYD | D5-12.3.2 | (SEQ ID NO: 810) | 188 |
| IVATI | D5-12.1.3 | (SEQ ID NO: 817) | 189 |
| yWLRL | D5-12.2.3 | (SEQ ID NO: 818) | 190 |
| SGYDY | D5-12.3.3 | (SEQ ID NO: 819) | 191 |
| VEMAT | D5-24.1.1 | (SEQ ID NO: 826) | 192 |
| yRWLQ | D5-24.2.1 | (SEQ ID NO: 827) | 193 |
| RDGYN | D5-24.3.1 | (SEQ ID NO: 828) | 194 |
| EMATI | D5-24.1.2 | (SEQ ID NO: 835) | 195 |
| RWLQL | D5-24.2.2 | (SEQ ID NO: 836) | 196 |
| DGYNY | D5-24.3.2 | (SEQ ID NO: 837) | 197 |
| EYSSS | D6-6.1.1 | (SEQ ID NO: 844) | 198 |
| SIAAR | D6-6.2.1 | (SEQ ID NO: 845) | 199 |
| VyQLV | D6-6.3.1 | (SEQ ID NO: 846) | 200 |
| YSSSS | D6-6.1.2 | (SEQ ID NO: 851) | 201 |
| GYSSS | D6-13.1.1 | (SEQ ID NO: 856) | 202 |
| GIAAA | D6-13.2.1 | (SEQ ID NO: 857) | 203 |
| VyQQL | D6-13.3.1 | (SEQ ID NO: 858) | 204 |
| YSSSW | D6-13.1.2 | (SEQ ID NO: 864) | 205 |
| IAAAG | D6-13.2.2 | (SEQ ID NO: 865) | 206 |
| yQQLV | D6-13.3.2 | (SEQ ID NO: 866) | 207 |
| SSSWY | D6-13.1.3 | (SEQ ID NO: 871) | 208 |
| GYSSG | D6-19.1.1 | (SEQ ID NO: 875) | 209 |
| GIAVA | D6-19.2.1 | (SEQ ID NO: 876) | 210 |
| VyQWL | D6-19.3.1 | (SEQ ID NO: 877) | 211 |
| YSSGW | D6-19.1.2 | (SEQ ID NO: 884) | 212 |
| IAVAG | D6-19.2.2 | (SEQ ID NO: 885) | 213 |
| yQWLV | D6-19.3.2 | (SEQ ID NO: 886) | 214 |
| SSGWY | D6-19.1.3 | (SEQ ID NO: 891) | 215 |

TABLE 14
All hexamers that can be extracted from human
D segments

| | | | |
|---|---|---|---|
| GIVGAT | D1-26.1.1 | (SEQ ID NO: 282) | 1 |
| VyWELL | D1-26.2.1 | (SEQ ID NO: 283) | 2 |
| YSGSYY | D1-26.3.1 | (SEQ ID NO: 284) | 3 |
| RILyyY | D2-2.1.1 | (SEQ ID NO: 300) | 4 |
| GYCSST | D2-2.2.1 | (SEQ ID NO: 301) | 5 |
| DIVVVP | D2-2.3.1 | (SEQ ID NO: 302) | 6 |
| ILyyYQ | D2-2.1.2 | (SEQ ID NO: 309) | 7 |
| YCSSTS | D2-2.2.2 | (SEQ ID NO: 310) | 8 |
| IVVVPA | D2-2.3.2 | (SEQ ID NO: 311) | 9 |
| LyyYQL | D2-2.1.3 | (SEQ ID NO: 318) | 10 |
| CSSTSC | D2-2.2.3 | (SEQ ID NO: 319) | 11 |
| VVVPAA | D2-2.3.3 | (SEQ ID NO: 320) | 12 |
| yyYQLL | D2-2.1.4 | (SEQ ID NO: 327) | 13 |
| SSTSCY | D2-2.2.4 | (SEQ ID NO: 328) | 14 |
| VVPAAI | D2-2.3.4 | (SEQ ID NO: 329) | 15 |
| yYQLLY | D2-2.1.5 | (SEQ ID NO: 336) | 16 |
| STSCYT | D2-2.2.5 | (SEQ ID NO: 337) | 17 |
| RILYyW | D2-8.1.1 | (SEQ ID NO: 351) | 18 |
| GYCTNG | D2-8.2.1 | (SEQ ID NO: 352) | 19 |
| DIVLMV | D2-8.3.1 | (SEQ ID NO: 353) | 20 |
| ILYyWC | D2-8.1.2 | (SEQ ID NO: 360) | 21 |
| YCTNGV | D2-8.2.2 | (SEQ ID NO: 361) | 22 |
| IVLMVY | D2-8.3.2 | (SEQ ID NO: 362) | 23 |
| LYyWCM | D2-8.1.3 | (SEQ ID NO: 369) | 24 |
| CTNGVC | D2-8.2.3 | (SEQ ID NO: 370) | 25 |
| VLMVYA | D2-8.3.3 | (SEQ ID NO: 371) | 26 |
| YyWCML | D2-8.1.4 | (SEQ ID NO: 378) | 27 |
| TNGVCY | D2-8.2.4 | (SEQ ID NO: 379) | 28 |
| LMVYAI | D2-8.3.4 | (SEQ ID NO: 380) | 29 |
| yWCMLY | D2-8.1.5 | (SEQ ID NO: 387) | 30 |
| NGVCYT | D2-8.2.5 | (SEQ ID NO: 388) | 31 |
| RILyWW | D2-15.1.1 | (SEQ ID NO: 398) | 32 |
| GYCSGG | D2-15.2.1 | (SEQ ID NO: 399) | 33 |
| DIVVVV | D2-15.3.1 | (SEQ ID NO: 400) | 34 |
| ILyWWy | D2-15.1.2 | (SEQ ID NO: 406) | 35 |
| YCSGGS | D2-15.2.2 | (SEQ ID NO: 407) | 36 |
| IVVVVA | D2-15.3.2 | (SEQ ID NO: 408) | 37 |
| LyWWyL | D2-15.1.3 | (SEQ ID NO: 415) | 38 |
| CSGGSC | D2-15.2.3 | (SEQ ID NO: 416) | 39 |
| VVVVAA | D2-15.3.3 | (SEQ ID NO: 417) | 40 |
| yWWyLL | D2-15.1.4 | (SEQ ID NO: 424) | 41 |
| SGGSCY | D2-15.2.4 | (SEQ ID NO: 425) | 42 |
| VVVAAT | D2-15.3.4 | (SEQ ID NO: 426) | 43 |
| WWyLLL | D2-15.1.5 | (SEQ ID NO: 433) | 44 |
| GGSCYS | D2-15.2.5 | (SEQ ID NO: 434) | 45 |
| SILWWw | D2-21.1.1 | (SEQ ID NO: 448) | 46 |
| AYCGGD | D2-21.2.1 | (SEQ ID NO: 449) | 47 |
| HIVVVT | D2-21.3.1 | (SEQ ID NO: 450) | 48 |
| ILWWwL | D2-21.1.2 | (SEQ ID NO: 456) | 49 |
| YCGGDC | D2-21.2.2 | (SEQ ID NO: 457) | 50 |
| IVVVTA | D2-21.3.2 | (SEQ ID NO: 458) | 51 |
| LWWwLL | D2-21.1.3 | (SEQ ID NO: 465) | 52 |
| CGGDCY | D2-21.2.3 | (SEQ ID NO: 466) | 53 |
| VVVTAI | D2-21.3.3 | (SEQ ID NO: 467) | 54 |
| WWwLLF | D2-21.1.4 | (SEQ ID NO: 474) | 55 |
| GGDCYS | D2-21.2.4 | (SEQ ID NO: 475) | 56 |
| VLRFLE | D3-3.1.1 | (SEQ ID NO: 489) | 57 |
| YYDFWS | D3-3.2.1 | (SEQ ID NO: 490) | 58 |
| ITIFGV | D3-3.3.1 | (SEQ ID NO: 491) | 59 |
| LRFLEW | D3-3.1.2 | (SEQ ID NO: 498) | 60 |
| YDFWSG | D3-3.2.2 | (SEQ ID NO: 499) | 61 |
| TIFGVV | D3-3.3.2 | (SEQ ID NO: 500) | 62 |
| RFLEWL | D3-3.1.3 | (SEQ ID NO: 507) | 63 |
| DFWSGY | D3-3.2.3 | (SEQ ID NO: 508) | 64 |
| IFGVVI | D3-3.3.3 | (SEQ ID NO: 509) | 65 |
| FLEWLL | D3-3.1.4 | (SEQ ID NO: 516) | 66 |
| FWSGYY | D3-3.2.4 | (SEQ ID NO: 517) | 67 |
| FGVVII | D3-3.3.4 | (SEQ ID NO: 518) | 68 |
| LEWLLY | D3-3.1.5 | (SEQ ID NO: 525) | 69 |
| WSGYYT | D3-3.2.5 | (SEQ ID NO: 526) | 70 |
| VLRYFD | D3-9.1.1 | (SEQ ID NO: 539) | 71 |
| YYDILT | D3-9.2.1 | (SEQ ID NO: 540) | 72 |
| ITIFyL | D3-9.3.1 | (SEQ ID NO: 541) | 73 |
| LRYFDW | D3-9.1.2 | (SEQ ID NO: 548) | 74 |
| YDILTG | D3-9.2.2 | (SEQ ID NO: 549) | 75 |
| TIFyLV | D3-9.3.2 | (SEQ ID NO: 550) | 76 |
| RYFDWL | D3-9.1.3 | (SEQ ID NO: 557) | 77 |
| DILTGY | D3-9.2.3 | (SEQ ID NO: 558) | 78 |
| IFyLVI | D3-9.3.3 | (SEQ ID NO: 559) | 79 |
| YFDWLL | D3-9.1.4 | (SEQ ID NO: 566) | 80 |
| ILTGYY | D3-9.2.4 | (SEQ ID NO: 567) | 81 |
| FyLVII | D3-9.3.4 | (SEQ ID NO: 568) | 82 |
| FDWLLy | D3-9.1.5 | (SEQ ID NO: 575) | 83 |
| LTGYYN | D3-9.2.5 | (SEQ ID NO: 576) | 84 |
| VLLWFG | D3-10.1.1 | (SEQ ID NO: 590) | 85 |
| YYYGSG | D3-10.2.1 | (SEQ ID NO: 591) | 86 |
| ITMVRG | D3-10.3.1 | (SEQ ID NO: 592) | 87 |
| LLWFGE | D3-10.1.2 | (SEQ ID NO: 599) | 88 |
| YYGSGS | D3-10.2.2 | (SEQ ID NO: 600) | 89 |
| TMVRGV | D3-10.3.2 | (SEQ ID NO: 601) | 90 |
| LWFGEL | D3-10.1.3 | (SEQ ID NO: 608) | 91 |
| YGSGSY | D3-10.2.3 | (SEQ ID NO: 609) | 92 |
| MVRGVI | D3-10.3.3 | (SEQ ID NO: 610) | 93 |
| WFGELL | D3-10.1.4 | (SEQ ID NO: 617) | 94 |
| GSGSYY | D3-10.2.4 | (SEQ ID NO: 618) | 95 |
| VRGVII | D3-10.3.4 | (SEQ ID NO: 619) | 96 |
| FGELLy | D3-10.1.5 | (SEQ ID NO: 624) | 97 |
| SGSYYN | D3-10.2.5 | (SEQ ID NO: 625) | 98 |
| VLwLRL | D3-16.1.1 | (SEQ ID NO: 638) | 99 |
| YYDYVW | D3-16.2.1 | (SEQ ID NO: 639) | 100 |
| IMITFG | D3-16.3.1 | (SEQ ID NO: 640) | 101 |
| LwLRLG | D3-16.1.2 | (SEQ ID NO: 647) | 102 |
| YDYVWG | D3-16.2.2 | (SEQ ID NO: 648) | 103 |
| MITFGG | D3-16.3.2 | (SEQ ID NO: 649) | 104 |
| wLRLGE | D3-16.1.3 | (SEQ ID NO: 656) | 105 |
| DYVWGS | D3-16.2.3 | (SEQ ID NO: 657) | 106 |
| ITFGGV | D3-16.3.3 | (SEQ ID NO: 658) | 107 |
| LRLGEL | D3-16.1.4 | (SEQ ID NO: 665) | 108 |
| YVWGSY | D3-16.2.4 | (SEQ ID NO: 666) | 109 |
| TFGGVI | D3-16.3.4 | (SEQ ID NO: 667) | 110 |
| RLGELS | D3-16.1.5 | (SEQ ID NO: 674) | 111 |
| VWGSYR | D3-16.2.5 | (SEQ ID NO: 675) | 112 |
| FGGVIV | D3-16.3.5 | (SEQ ID NO: 676) | 113 |
| LGELSL | D3-16.1.6 | (SEQ ID NO: 683) | 114 |
| WGSYRY | D3-16.2.6 | (SEQ ID NO: 684) | 115 |
| GGVIVI | D3-16.3.6 | (SEQ ID NO: 685) | 116 |
| GELSLY | D3-16.1.7 | (SEQ ID NO: 692) | 117 |
| GSYRYT | D3-16.2.7 | (SEQ ID NO: 693) | 118 |
| VLLwyy | D3-22.1.1 | (SEQ ID NO: 707) | 119 |
| YYYDSS | D3-22.2.1 | (SEQ ID NO: 708) | 120 |
| ITMIVV | D3-22.3.1 | (SEQ ID NO: 709) | 121 |
| LLwyyW | D3-22.1.2 | (SEQ ID NO: 716) | 122 |
| YYDSSG | D3-22.2.2 | (SEQ ID NO: 717) | 123 |
| TMIVVV | D3-22.3.2 | (SEQ ID NO: 718) | 124 |
| LwyyWL | D3-22.1.3 | (SEQ ID NO: 725) | 125 |
| YDSSGY | D3-22.2.3 | (SEQ ID NO: 726) | 126 |
| MIVVVI | D3-22.3.3 | (SEQ ID NO: 727) | 127 |
| wyyWLL | D3-22.1.4 | (SEQ ID NO: 733) | 128 |
| DSSGYY | D3-22.2.4 | (SEQ ID NO: 734) | 129 |
| IVVVIT | D3-22.3.4 | (SEQ ID NO: 735) | 130 |
| yyWLLL | D3-22.1.5 | (SEQ ID NO: 742) | 131 |
| SSGYYY | D3-22.2.5 | (SEQ ID NO: 743) | 132 |
| wLRWyL | D4-23.1.1 | (SEQ ID NO: 769) | 133 |
| DYGGNS | D4-23.2.1 | (SEQ ID NO: 770) | 134 |
| VDTAMV | D5-5.1.1 | (SEQ ID NO: 784) | 135 |
| WIQLWL | D5-5.2.1 | (SEQ ID NO: 785) | 136 |
| GYSYGY | D5-5.3.1 | (SEQ ID NO: 786) | 137 |
| VDIVAT | D5-12.1.1 | (SEQ ID NO: 802) | 138 |
| WIyWLR | D5-12.2.1 | (SEQ ID NO: 803) | 139 |
| GYSGYD | D5-12.3.1 | (SEQ ID NO: 804) | 140 |
| DIVATI | D5-12.1.2 | (SEQ ID NO: 811) | 141 |
| IyWLRL | D5-12.2.2 | (SEQ ID NO: 812) | 142 |
| YSGYDY | D5-12.3.2 | (SEQ ID NO: 813) | 143 |
| VEMATI | D5-24.1.1 | (SEQ ID NO: 829) | 144 |
| yRWLQL | D5-24.2.1 | (SEQ ID NO: 830) | 145 |
| RDGYNY | D5-24.3.1 | (SEQ ID NO: 831) | 146 |
| EYSSSS | D6-6.1.1 | (SEQ ID NO: 847) | 147 |
| GYSSSW | D6-13.1.1 | (SEQ ID NO: 859) | 148 |
| GIAAAG | D6-13.2.1 | (SEQ ID NO: 860) | 149 |
| VyQQLV | D6-13.3.1 | (SEQ ID NO: 861) | 150 |
| YSSSWY | D6-13.1.2 | (SEQ ID NO: 867) | 151 |
| GYSSGW | D6-19.1.1 | (SEQ ID NO: 878) | 152 |
| GIAVAG | D6-19.2.1 | (SEQ ID NO: 879) | 153 |
| VyQWLV | D6-19.3.1 | (SEQ ID NO: 880) | 154 |
| YSSGWY | D6-19.1.2 | (SEQ ID NO: 887) | 155 |

### Example 3: CDR3 of Length 6-20

Insertion of D segments into synthetic HC CDR3s can lead to greater stability and lower immunogenicity. Libraries are designed at the amino-acid level by joining a VH to an optional filler of some length which is joined to a D segment an optional second filler and a JH. For libraries of length six or eight, a full-length JH may follow VH and a short filler. Where D segments are used, the D segments D2-2 (RF 2), D2-8 (RF 2), D2-15 (RF 2), D2-21 (RF 2), D3-16 (RF 2), D3-22 (RF 2), D3-3 (RF-2), D3-9 (RF 2), D3-10 (RF 2), D1-26 (RF 3), D4-11 (RF 2), D4-4 (RF 2), D5-5 (RF 3), D5-12 (RF 3), D5-18 (RF 3), D6-6 (RF1), D6-13 (RF 1), and D6-19 (RF 1) are preferred.

Once the parental amino-acid sequence has been designed, it can be diversified in several ways: error-prone PCR, wobbling, and dobbling. Table 14 shows a number of hexamers that can be derived from human D regions. In one embodiment, the hexamers that contain cysteine residues are excluded. In one embodiment, the fragments of D regions that contain stops are excluded. In one embodiment, any TAG codon found in the D region is replaced by a codon picked from the set comprising TCG, TTG, TGG, CAG, AAG, TAT, and GAG. In one embodiment, any TAA codon found in the D region is replaced by a codon picked form the set comprising TCA, TTA, CAA, AAA, TAT, and GAA. In one embodiment, any TGA of the D region is replaced by a codon picked from the set comprising TGG, TCA, TTA, AGA, and GGA.

Table 21 shows exemplary parental amino-acid sequences for CDR3s from 6 to 20 amino acids. These parental sequences can be combined with diversity in HC CDR1 and CDR2 to form a library. The utility is likely to improve if the CDR3 regions are diversified by, for example, wobbling, dobbling, or error-prone PCR of the CDR3s. In Table 21, sequence 6a comprises the end of VH from 3-23 fused to whole JH1. Sequence 6b contains the end of 3-23 joined to a Y joined to D4-17 (RF 2) joined to the FR4 region of JH1. Sequence 6c contains the end of 3-23 followed by D5-5 (RF 3) followed by the FR4 part of JH1. Sequence 6d contains the end of 3-23 joined to SY joined to the whole JH4. Table 21 shows the level of doping that would be appropriate for the wobbling of the CDR3; other levels could be used as well. Other D regions or fragments of D regions could be used. Other JH sequences could be used.

TABLE 21
Parental amino-acid sequences for
HC CDR3s of 6-20 AAs.

| | | level | | SEQ |
|---|---|---|---|---|
| | Parental | of | | ID |
| Length | sequence | doping | Comment | NO: |
| 6a | yycakAEYFQH | 70:10:10:10 | JH1(whole) | 226 |
| | wgqgtlvtvss | | | |
| 6b | yycakYDYGDY | 70:10:10:10 | Y::D4-17 | 227 |
| | wgqgtlvtvss | | (2)::FR4 | |
| | | | of JH1 | |
| 6c | yycakGYSYGY | 70:10:10:10 | D5-5(3):: | 228 |
| | wgqgtlvtvss | | FR4 of JH1 | |
| 6d | yycakSYYFDY | 70:10:10:10 | SY::JH4 | 229 |
| | wgqgtlvtvss | | (whole) | |
| 8a | yycakYYAEYFQ | 73:9:9:9 | YY:JH1 | 230 |
| | Hwgqgtlvtvss | | (whole) | |
| 8b | yycakYGYSSSW | 73:9:9:9 | Y::D6-13 | 231 |
| | Ywgqgtlvtvss | | (1)::FR4 | |
| | | | of JH1 | |
| 8c | yycakYGDYYFD | 73:9:9:9 | D4-17(2) | 232 |
| | Ywgqgtlvtvss | | [2-5]:: | |
| | | | JH4(whole) | |
| 10a | yycakYYYDSSG | 73:9:9:9 | D3-22(2):: | 233 |
| | YYYwgqgtlvtv | | Fr4 of JH1 | |
| | ss | | | |
| 10b | yycakGYcSSTS | 73:9:9:9 | D2-2(2):: | 234 |
| | cYTwgqgtlvtv | | Fr4 of JH1 | |
| | ss | | | |
| 10c | yycakYYSSAEY | 73:9:9:9 | YYSS::JH1 | 235 |
| | FQHwgqgtlvtv | | (whole) | |
| | ss | | | |
| 10d | yycakGYSYGYY | 73:9:9:9 | D5-5(3):: | 236 |
| | FDYwgqgtlvtv | | JH4(whole) | |
| | ss | | | |
| 12a | yycakYYYDSSG | 85:5:5:5 | D3-22(2):: | 237 |
| | YYYQHwgqgtlv | | QH::Fr4 | |
| | tvss | | of JH1 | |
| 12b | yycakGYcSSTS | 85:5:5:5 | D2-2(2):: | 238 |
| | cYTQHwgqgtlv | | QH::Fr4 | |
| | tvss | | of JH1 | |
| 12c | yycakYYSSYSA | 85:5:5:5 | YYSSYS:: | 239 |
| | EYFQHwgqgtlv | | JH1(whole) | |
| | tvss | | | |
| 12d | yycakYYDYVWG | 85:5:5:5 | D3-16(2):: | 240 |
| | SYRYTwgqgtlv | | Fr of JH1 | |
| | tvss | | | |
| 12e | yycakGYSYGYY | 85:5:5:5 | D5-5(3):: | 241 |
| | WYFDLwgrgtlv | | JH2(whole) | |
| | tvss | | | |
| 14a | yycakYYYDSSG | 73:9:9:9 | D3-22(2):: | 242 |
| | YYYYFQHwgqgt | | YFQH::Fr | |
| | lvtvss | | of JH1 | |
| 14b | yycakGYcSSTS | 73:9:9:9 | D2-2(2):: | 243 |
| | cYTYFQHwgqgt | | YFQH::Fr | |
| | lvtvss | | of JH1 | |
| 14c | yycakSYGYcSS | 73:9:9:9 | SY::D2-2 | 244 |
| | TScYTQHwgqgt | | (2)::QH:: | |
| | lvtvss | | Fr of JH1 | |
| 14d | yycakSYYYSSY | 73:9:9:9 | SYYYSSYS:: | 245 |
| | SAEYFQHwgqgt | | JH1(whole) | |
| | lvtvss | | | |
| 14e | yycakAYcGGDc | 73:9:9:9 | D2-21(2):: | 246 |
| | YSNWFDPwgqgt | | JH5(whole) | |
| | lvtvss | | | |
| 16a | yycakYYYDSSG | 73:9:9:9 | D3-22(2):: | 247 |
| | YYYAEYFQHwgq | | JH1(whole) | |
| | gtlvtvss | | | |
| 16b | yycakGYcSSTS | 73:9:9:9 | D2-2(2):: | 248 |
| | cYTAEYFQHwgq | | JH1(whole) | |
| | gtlvtvss | | | |
| 16c | yycakSYYSYSS | 73:9:9:9 | SYYSYSSYYS:: | 249 |
| | YYSAEYFQHwgq | | JH1(whole) | |
| | gtlvtvss | | | |
| 16d | yycakSYSYGYc | 73:9:9:9 | SYSY::D2-2 | 250 |
| | SSTScYTQHwgq | | (2)::QH::Fr | |
| | gtlvtvss | | JH1 | |
| 20a | yycakYSSYYYY | 73:9:9:9 | YSSY::D3- | 251 |
| | DSSGYYYAEYFQ | | 22(2)::JH1 | |
| | Hwgqgtlvtvss | | (whole) | |
| 20b | yycakSYYSGYc | 73:9:9:9 | SYYS::D2- | 252 |
| | SSTScYTAEYFQ | | 2(2)::JH1 | |
| | Hwgqgtlvtvss | | (whole) | |
| 20c | yycakSGYcSST | 73:9:9:9 | S::D2-2(2):: | 253 |
| | ScYTYYSAEYFQ | | YYS::JH1 | |
| | Hwgqgtlvtvss | | (whole) | |
| 20d | yycakYYYYDYV | 73:9:9:9 | Y::D3-16 | 254 |
| | WGSYRYTSNWFD | | (2)::S::JH5 | |
| | Pwgqgtlvtvss | | (whole) | |
| 20e | yycakYYYYDYV | 73:9:9:9 | Y::D3-16 | 255 |
| | WGSYRYTSSYFD | | (2)::SS::JH4 | |
| | Ywgqgtlvtvss | | (whole) | |

TABLE 22
HC display cassette

| | |
|---|---|
| | Signal for VH-CH1-IIIstump |
| | 1 2 3 4 5 6 7 8 9 10 11 12 13 14 15 |
| | M K Y L L P T A A A G L L L L |
| 946 | atg aaa tac cta ttg cct acg gca gcc gct gga ttg tta tta ctc |
| | 16 17 18 19 20 21 22 |
| | A A Q P A M A |
| 991 | gcG GCC cag ccG GCC atg gcc |
| | SfiI............. |
| | NgoMI...(1/2) |
| | NcoI.... |
| | VH |
| | FR1(DP47/V3-23)-------------- |
| | 1 2 3 4 5 6 7 8 |
| | E V Q L L E S G |
| 1012 | gaa\|gtt\|CAA\|TTG\|tta\|gag\|tct\|ggt\| |
| | \| MfeI \| |
| | ---------------FR1------------------------------------------- |
| | 9 10 11 12 13 14 15 16 17 18 19 20 21 22 23 |
| | G G L V Q P G G S L R L S C A |
| 1036 | \|ggc\|ggt\|ctt\|gtt\|cag\|cct\|ggt\|ggt\|tct\|tta\|cgt\|ctt\|tct\|tgc\|gct\| |
| | ----FR1-------------------->\|...CDR1............\|---FR2------ |
| | 24 25 26 27 28 29 30 31 32 33 34 35 36 37 38 |
| | A S G F T F S S Y A M S W V R |
| 1081 | \|gct\|TCC\|GGA\|ttc\|act\|ttc\|tct\|tCG\|TAC\|Gct\|atg\|tct\|tgg\|gtt\|cgC\| |
| | \| BspEI \| \| BsiWI\| \|BstXI. |
| | --------FR2-------------------------------->\|...CDR2......... |
| | 39 40 41 42 43 44 45 46 47 48 49 50 51 52 52a |
| | Q A P G K G L E W V S A I S G |
| 1126 | \|CAa\|gct\|ccT\|GGt\|aaa\|ggt\|ttg\|gag\|tgg\|gtt\|tct\|gct\|atc\|tct\|ggt\| |
| | ...BstXI \| |
| | .....CDR2...........................................\|---FR3--- |
| | 53 54 55 56 57 58 59 60 61 62 63 64 65 66 67 |
| | S G G S T Y Y A D S V K G R F |
| 1171 | \|tct\|ggt\|ggc\|agt\|act\|tac\|tat\|gct\|gac\|tcc\|gtt\|aaa\|ggt\|cgc\|ttc\| |
| | --------FR3-------------------------------------------------- |
| | 68 69 70 71 72 73 74 75 76 77 78 79 80 81 82 |
| | T I S R D N S K N T L Y L Q M |
| 1216 | \|act\|atc\|TCT\|AGA\|gac\|aac\|tct\|aag\|aat\|act\|ctc\|tac\|ttg\|cag\|atg\| |
| | \| XbaI \| |
| | ---FR3----------------------------------------------------->\| |
| | 82a 82b 82c 83 84 85 86 87 88 89 90 91 92 93 94 |
| | N S L R A E D T A V Y Y C A K |
| 1261 | \|aac\|agC\|TTA\|AGg\|gct\|gag\|gac\|aCT\|GCA\|Gtc\|tac\|tat\|tgc\|gct\|aaa\| |
| | \|AflII \| \| PstI \| (2/2) |
| | .......CDR3.................\|----FR4------------------------- |
| | 95 96 97 98 98a 98b 98c 99 100 101 102 103 104 105 106 |
| | D Y E G T G Y A F D I W G Q G |
| 1306 | \|gac\|tat\|gaa\|ggt\|act\|ggt\|tat\|gct\|ttc\|gaC\|ATA\|TGg\|ggt\|caa\|ggt\| |
| | \| NdeI \| |
| | --------------FR4---------->\| |
| | 107 108 109 110 111 112 113 |
| | T M V T V S S |
| 1351 | \|act\|atG\|GTC\|ACC\|gtc\|tct\|agt |
| | \| BstEII \| c tcg ag = XhoI. |
| | CH1 |
| | A S T K G P S V F P L A P S S |
| 1372 | gcc tcc acc aag ggc cca tcg gtc ttc ccG CTA GCa ccc tcc tcc |
| | NheI.... |
| | 151 152 153 154 155 156 157 158 159 160 161 162 163 164 165 |
| | K S T S G G T A A L G C L V K |
| 1417 | aag agc acc tct ggg ggc aca gcg gcc ctg ggc tgc ctg gtc aag |
| | 166 167 168 169 170 171 172 173 174 175 176 177 178 179 180 |
| | D Y F P E P V T V S W N S G A |
| 1462 | gac tac ttc ccc gaa ccg gtg acg gtg tcg tgg aac tca ggc gcc |
| | 181 182 183 184 185 186 187 188 189 190 191 192 193 194 195 |
| | L T S G V H T F P A V L Q S S |
| 1507 | ctg acc agc ggc gtc cac acc ttc ccg gct gtc cta cag tcc tca |
| | 196 197 198 199 200 201 202 203 204 205 206 207 208 209 210 |
| | G L Y S L S S V V T V P S S S |
| 1552 | gga ctc tac tcc ctc ago agc gta gtg acc gtg ccc tCC Agc agc |
| | BstXI........ |

| | |
|---|---|
| | 211 212 213 214 215 216 217 218 219 220 221 222 223 224 225 |
| | L G T Q T Y I C N V N H K P S |
| 1597 | tTG Ggc acc cag acc tac atc tgc aac gtg aat cac aag ccc agc |
| | BstXI........ |
| | 226 227 228 229 230 231 232 233 234 235 236 237 238 |
| | N T K V D K K V E P K S C |
| 1642 | aac acc aag gtg gac aaG AAA GTT GAG CCC AAA TCT TGT |
| | 139 140 141 His tag.............. cMyc tag...................... |
| | A A A H H H H H H G A A E Q K L I |
| 1681 | GCG GCC GCa cat cat cat cac cat cac ggg gcc gca gaa caa aaa ctc atc |
| | NotI...... |
| | EagI.... |
| | ................................... |
| | S E E D L N G A A E A S S A S N A S |
| 1732 | tca gaa gag gat ctg aat ggg GCC gca gaG GCt agt tct gct agt aAC GCG Tct |
| | BglI..........(3/4) MluI.... |
| | Domain 3 (IIIstump)------------------------------------------------- |
| | S G D F D Y E K M A N A N K G A |
| 1786 | tcc ggt gat ttt gat tat gaa aag atg gca aac gct aat aag ggg gct |
| | M T E N A D E N A L Q S D A K G |
| 1834 | atg acc gaa aat gcc gat gaa aac gcg cta cag tct gac gct aaa ggc |
| | K L D S V A T D Y G A A I D G F |
| 1882 | aaa ctt gat tct gtc gct act gat tac ggt gct gct atc gat ggt ttc |
| | I G D V S G L A N G N G A T G D |
| 1930 | att ggt gac gtt tcc ggc ctt gct aat ggt aat ggt gct act ggt gat |
| | F A G S N S Q M A Q V G D G D N |
| 1978 | ttt gct ggc tct aat tcc caa atg gct caa gtc ggt gac ggt gat aat |
| | S P L M N N F R Q Y L P S L P Q |
| 2026 | tca cct tta atg aat aat ttc cgt caa tat tta cct tcc ctc cct caa |
| | S V E C R P F V F G A G K P Y E |
| 2074 | tcg gtt gaa tgt cgc cct ttt gtc ttt ggc gct ggt aaa cca tat gaa |
| | F S I D C D K I N L F R |
| 2122 | ttt tct att gat tgt gac aaa ata aac tta ttc cgt |
| | End Domain 3 |
| | G V F A F L L Y V A T F M Y V F140 |
| 2158 | ggt gtc ttt gcg ttt ctt tta tat gtt gcc acc ttt atg tat gta ttt |
| | start transmembrane segment |
| | S T F A N I L |
| 2206 | tct acg ttt gct aac ata ctg |
| | R N K E S (SEQ ID NO: 892) |
| 2227 | cgt aat aag gag tct TAA tga aAC GCG Tga tga GAATTC (SEQ ID NO: 893) |
| | Intracellular anchor. MluI.... EcoRI. |

TABLE 25
The DNA sequence of DY3F85LC containing a sample germline O12 kappa light
chain. The antibody sequences shown are of the form of actual antibody,
but have not been identified as binding to a particular antigen.
On each line, everything after an exclamation point (!) is commentary.
The DNA of DY3F85LC is SEQ ID NO: 27

!---------------------------------------------------------------------

| | |
|---|---|
| 1 | AATGCTACTA CTATTAGTAG AATTGATGCC ACCTTTTCAG CTCGCGCCCC AAATGAAAAT |
| 61 | ATAGCTAAAC AGGTTATTGA CCATTTGCGA AATGTATCTA ATGGTCAAAC TAAATCTACT |
| 121 | CGTTCGCAGA ATTGGGAATC AACTGTTATA TGGAATGAAA CTTCCAGACA CCGTACTTTA |
| 181 | GTTGCATATT TAAAACATGT TGAGCTACAG CATTATATTC AGCAATTAAG CTCTAAGCCA |
| 241 | TCCGCAAAAA TGACCTCTTA TCAAAAGGAG CAATTAAAGG TACTCTCTAA TCCTGACCTG |
| 301 | TTGGAGTTTG CTTCCGGTCT GGTTCGCTTT GAAGCTCGAA TTAAAACGCG ATATTTGAAG |
| 361 | TCTTTCGGGC TTCCTCTTAA TCTTTTTGAT GCAATCCGCT TTGCTTCTGA CTATAATAGT |
| 421 | CAGGGTAAAG ACCTGATTTT TGATTTATGG TCATTCTCGT TTTCTGAACT GTTTAAAGCA |
| 481 | TTTGAGGGGG ATTCAATGAA TATTTATGAC GATTCCGCAG TATTGGACGC TATCCAGTCT |
| 541 | AAACATTTTA CTATTACCCC CTCTGGCAAA ACTTCTTTTG CAAAAGCCTC TCGCTATTTT |
| 601 | GGTTTTTATC GTCGTCTGGT AAACGAGGGT TATGATAGTG TTGCTCTTAC TATGCCTCGT |
| 661 | AATTCCTTTT GGCGTTATGT ATCTGCATTA GTTGAATGTG GTATTCCTAA ATCTCAACTG |
| 721 | ATGAATCTTT CTACCTGTAA TAATGTTGTT CCGTTAGTTC GTTTTATTAA CGTAGATTTT |
| 781 | TCTTCCCAAC GTCCTGACTG GTATAATGAG CCAGTTCTTA AAATCGCATA AGGTAATTCA |
| 841 | CAATGATTAA AGTTGAAATT AAACCATCTC AAGCCCAATT TACTACTCGT TCTGGTGTTT |
| 901 | CTCGTCAGGG CAAGCCTTAT TCACTGAATG AGCAGCTTTG TTACGTTGAT TTGGGTAATG |
| 961 | AATATCCGGT TCTTGTCAAG ATTACTCTTG ATGAAGGTCA GCCAGCCTAT GCGCCTGGTC |
| 1021 | TGTACACCGT TCATCTGTCC TCTTTCAAAG TTGGTCAGTT CGGTTCCCTT ATGATTGACC |
| 1081 | GTCTGCGCCT CGTTCCGGCT AAGTAACATG GAGCAGGTCG CGGATTTCGA CACAATTTAT |
| 1141 | CAGGCGATGA TACAAATCTC CGTTGTACTT TGTTTCGCGC TTGGTATAAT CGCTGGGGGT |
| 1201 | CAAAGATGAG TGTTTTAGTG TATTCTTTTG CCTCTTTCGT TTTAGGTTGG TGCCTTCGTA |
| 1261 | GTGGCATTAC GTATTTTACC CGTTTAATGG AAACTTCCTC ATGAAAAAGT CTTTAGTCCT |
| 1321 | CAAAGCCTCT GTAGCCGTTG CTACCCTCGT TCCGATGCTG TCTTTCGCTG CTGAGGGTGA |
| 1381 | CGATCCCGCA AAAGCGGCCT TTAACTCCCT GCAAGCCTCA GCGACCGAAT ATATCGGTTA |
| 1441 | TGCGTGGGCG ATGGTTGTTG TCATTGTCGG CGCAACTATC GGTATCAAGC TGTTTAAGAA |
| 1501 | ATTCACCTCG AAAGCAAGCT GATAAACCGA TACAATTAAA GGCTCCTTTT GGAGCCTTTT |
| 1561 | TTTTGGAGAT TTTCAACGTG AAAAAATTAT TATTCGCAAT TCCTTTAGTT GTTCCTTTCT |
| 1621 | ATTCTCACTC CGCTGAAACT GTTGAAAGTT GTTTAGCAAA ATCCCATACA GAAAATTCAT |
| 1681 | TTACTAACGT CTGGAAAGAC GACAAAACTT TAGATCGTTA CGCTAACTAT GAGGGCTGTC |
| 1741 | TGTGGAATGC TACAGGCGTT GTAGTTTGTA CTGGTGACGA AACTCAGTGT TACGGTACAT |
| 1801 | GGGTTCCTAT TGGGCTTGCT ATCCCTGAAA ATGAGGGTGG TGGCTCTGAG GGTGGCGGTT |
| 1861 | CTGAGGGTGG CGGTTCTGAG GGTGGCGGTA CTAAACCTCC TGAGTACGGT GATACACCTA |
| 1921 | TTCCGGGCTA TACTTATATC AACCCTCTCG ACGGCACTTA TCCGCCTGGT ACTGAGCAAA |
| 1981 | ACCCCGCTAA TCCTAATCCT TCTCTTGAGG AGTCTCAGCC TCTTAATACT TTCATGTTTC |
| 2041 | AGAATAATAG GTTCCGAAAT AGGCAGGGGG CATTAACTGT TTATACGGGC ACTGTTACTC |
| 2101 | AAGGCACTGA CCCCGTTAAA ACTTATTACC AGTACACTCC TGTATCATCA AAAGCCATGT |
| 2161 | ATGACGCTTA CTGGAACGGT AAATTCAGAG ACTGCGCTTT CCATTCTGGC TTTAATGAGG |
| 2221 | ATTTATTTGT TTGTGAATAT CAAGGCCAAT CGTCTGACCT GCCTCAACCT CCTGTCAATG |
| 2281 | CTGGCGGCGG CTCTGGTGGT GGTTCTGGTG GCGGCTCTGA GGGTGGTGGC TCTGAGGGTG |
| 2341 | GCGGTTCTGA GGGTGGCGGC TCTGAGGGAG GCGGTTCCGG TGGTGGCTCT GGTTCCGGTG |
| 2401 | ATTTTGATTA TGAAAAGATG GCAAACGCTA ATAAGGGGGC TATGACCGAA AATGCCGATG |
| 2461 | AAAACGCGCT ACAGTCTGAC GCTAAAGGCA AACTTGATTC TGTCGCTACT GATTACGGTG |
| 2521 | CTGCTATCGA TGGTTTCATT GGTGACGTTT CCGGCCTTGC TAATGGTAAT GGTGCTACTG |
| 2581 | GTGATTTTGC TGGCTCTAAT TCCCAAATGG CTCAAGTCGG TGACGGTGAT AATTCACCTT |
| 2641 | TAATGAATAA TTTCCGTCAA TATTTACCTT CCCTCCCTCA ATCGGTTGAA TGTCGCCCTT |
| 2701 | TTGTCTTTGG CGCTGGTAAA CCATATGAAT TTTCTATTGA TTGTGACAAA ATAAACTTAT |
| 2761 | TCCGTGGTGT CTTTGCGTTT CTTTTATATG TTGCCACCTT TATGTATGTA TTTTCTACGT |
| 2821 | TTGCTAACAT ACTGCGTAAT AAGGAGTCTT AATCATGCCA GTTCTTTTGG GTATTCCGTT |
| 2881 | ATTATTGCGT TTCCTCGGTT TCCTTCTGGT AACTTTGTTC GGCTATCTGC TTACTTTTCT |
| 2941 | TAAAAAGGGC TTCGGTAAGA TAGCTATTGC TATTTCATTG TTTCTTGCTC TTATTATTGG |
| 3001 | GCTTAACTCA ATTCTTGTGG GTTATCTCTC TGATATTAGC GCTCAATTAC CCTCTGACTT |
| 3061 | TGTTCAGGGT GTTCAGTTAA TTCTCCCGTC TAATGCGCTT CCCTGTTTTT ATGTTATTCT |
| 3121 | CTCTGTAAAG GCTGCTATTT TCATTTTTGA CGTTAAACAA AAAATCGTTT CTTATTTGGA |
| 3181 | TTGGGATAAA TAATATGGCT GTTTATTTTG TAACTGGCAA ATTAGGCTCT GGAAAGACGC |
| 3241 | TCGTTAGCGT TGGTAAGATT CAGGATAAAA TTGTAGCTGG GTGCAAAATA GCAACTAATC |
| 3301 | TTGATTTAAG GCTTCAAAAC CTCCCGCAAG TCGGGAGGTT CGCTAAAACG CCTCGCGTTC |
| 3361 | TTAGAATACC GGATAAGCCT TCTATATCTG ATTTGCTTGC TATTGGGCGC GGTAATGATT |
| 3421 | CCTACGATGA AAATAAAAAC GGCTTGCTTG TTCTCGATGA GTGCGGTACT TGGTTTAATA |
| 3481 | CCCGTTCTTG GAATGATAAG GAAAGACAGC CGATTATTGA TTGGTTTCTA CATGCTCGTA |
| 3541 | AATTAGGATG GGATATTATT TTTCTTGTTC AGGACTTATC TATTGTTGAT AAACAGGCGC |
| 3601 | GTTCTGCATT AGCTGAACAT GTTGTTTATT GTCGTCGTCT GGACAGAATT ACTTTACCTT |
| 3661 | TTGTCGGTAC TTTATATTCT CTTATTACTG GCTCGAAAAT GCCTCTGCCT AAATTACATG |
| 3721 | TTGGCGTTGT TAAATATGGC GATTCTCAAT TAAGCCCTAC TGTTGAGCGT TGGCTTTATA |
| 3781 | CTGGTAAGAA TTTGTATAAC GCATATGATA CTAAACAGGC TTTTTCTAGT AATTATGATT |
| 3841 | CCGGTGTTTA TTCTTATTTA ACGCCTTATT TATCACACGG TCGGTATTTC AAACCATTAA |
| 3901 | ATTTAGGTCA GAAGATGAAA TTAACTAAAA TATATTTGAA AAAGTTTTCT CGCGTTCTTT |
| 3961 | GTCTTGCGAT TGGATTTGCA TCAGCATTTA CATATAGTTA TATAACCCAA CCTAAGCCGG |
| 4021 | AGGTTAAAAA GGTAGTCTCT CAGACCTATG ATTTTGATAA ATTCACTATT GACTCTTCTC |
| 4081 | AGCGTCTTAA TCTAAGCTAT CGCTATGTTT TCAAGGATTC TAAGGGAAAA TTAATTAATA |
| 4141 | GCGACGATTT ACAGAAGCAA GGTTATTCAC TCACATATAT TGATTTATGT ACTGTTTCCA |
| 4201 | TTAAAAAAGG TAATTCAAAT GAAATTGTTA AATGTAATTA ATTTTGTTTT CTTGATGTTT |
| 4261 | GTTTCATCAT CTTCTTTTGC TCAGGTAATT GAAATGAATA ATTCGCCTCT GCGCGATTTT |
| 4321 | GTAACTTGGT ATTCAAAGCA ATCAGGCGAA TCCGTTATTG TTTCTCCCGA TGTAAAAGGT |
| 4381 | ACTGTTACTG TATATTCATC TGACGTTAAA CCTGAAAATC TACGCAATTT CTTTATTTCT |
| 4441 | GTTTTACGTG CAAATAATTT TGATATGGTA GGTTCTAACC CTTCCATAAT TCAGAAGTAT |
| 4501 | AATCCAAACA ATCAGGATTA TATTGATGAA TTGCCATCAT CTGATAATCA GGAATATGAT |
| 4561 | GATAATTCCG CTCCTTCTGG TGGTTTCTTT GTTCCGCAAA ATGATAATGT TACTCAAACT |
| 4621 | TTTAAAATTA ATAACGTTCG GGCAAAGGAT TTAATACGAG TTGTCGAATT GTTTGTAAAG |
| 4681 | TCTAATACTT CTAAATCCTC AAATGTATTA TCTATTGACG GCTCTAATCT ATTAGTTGTT |
| 4741 | AGTGCTCCTA AAGATATTTT AGATAACCTT CCTCAATTCC TTTCAACTGT TGATTTGCCA |
| 4801 | ACTGACCAGA TATTGATTGA GGGTTTGATA TTTGAGGTTC AGCAAGGTGA TGCTTTAGAT |
| 4861 | TTTTCATTTG CTGCTGGCTC TCAGCGTGGC ACTGTTGCAG GCGGTGTTAA TACTGACCGC |
| 4921 | CTCACCTCTG TTTTATCTTC TGCTGGTGGT TCGTTCGGTA TTTTTAATGG CGATGTTTTA |
| 4981 | GGGCTATCAG TTCGCGCATT AAAGACTAAT AGCCATTCAA AAATATTGTC TGTGCCACGT |
| 5041 | ATTCTTACGC TTTCAGGTCA GAAGGGTTCT ATCTCTGTTG GCCAGAATGT CCCTTTTATT |
| 5101 | ACTGGTCGTG TGACTGGTGA ATCTGCCAAT GTAAATAATC CATTTCAGAC GATTGAGCGT |
| 5161 | CAAAATGTAG GTATTTCCAT GAGCGTTTTT CCTGTTGCAA TGGCTGGCGG TAATATTGTT |
| 5221 | CTGGATATTA CCAGCAAGGC CGATAGTTTG AGTTCTTCTA CTCAGGCAAG TGATGTTATT |
| 5281 | ACTAATCAAA GAAGTATTGC TACAACGGTT AATTTGCGTG ATGGACAGAC TCTTTTACTC |
| 5341 | GGTGGCCTCA CTGATTATAA AAACACTTCT CAGGATTCTG GCGTACCGTT CCTGTCTAAA |
| 5401 | ATCCCTTTAA TCGGCCTCCT GTTTAGCTCC CGCTCTGATT CTAACGAGGA AAGCACGTTA |
| 5461 | TACGTGCTCG TCAAAGCAAC CATAGTACGC GCCCTGTAGC GGCGCATTAA GCGCGGCGGG |
| 5521 | TGTGGTGGTT ACGCGCAGCG TGACCGCTAC ACTTGCCAGC GCCCTAGCGC CCGCTCCTTT |
| 5581 | CGCTTTCTTC CCTTCCTTTC TCGCCACGTT CGCCGGCTTT CCCCGTCAAG CTCTAAATCG |
| 5641 | GGGGCTCCCT TTAGGGTTCC GATTTAGTGC TTTACGGCAC CTCGACCCCA AAAAACTTGA |
| 5701 | TTTGGGTGAT GGTTCACGTA GTGGGCCATC GCCCTGATAG ACGGTTTTTC GCCCTTTGAC |
| 5761 | GTTGGAGTCC ACGTTCTTTA ATAGTGGACT CTTGTTCCAA ACTGGAACAA CACTCAACCC |
| 5821 | TATCTCGGGC TATTCTTTTG ATTTATAAGG GATTTTGCCG ATTTCGGAAC CACCATCAAA |
| 5881 | CAGGATTTTC GCCTGCTGGG GCAAACCAGC GTGGACCGCT TGCTGCAACT CTCTCAGGGC |
| 5941 | CAGGCGGTGA AGGGCAATCA GCTGTTGCCC GTCTCACTGG TGAAAAGAAA AACCACCCTG |
| 6001 | GATCCAAGCT TGCAGGTGGC ACTTTTCGGG GAAATGTGCG CGGAACCCCT ATTTGTTTAT |
| 6061 | TTTTCTAAAT ACATTCAAAT ATGTATCCGC TCATGAGACA ATAACCCTGA TAAATGCTTC |
| 6121 | AATAATATTG AAAAAGGAAG AGTATGAGTA TTCAACATTT CCGTGTCGCC CTTATTCCCT |
| 6181 | TTTTTGCGGC ATTTTGCCTT CCTGTTTTTG CTCACCCAGA AACGCTGGTG AAAGTAAAAG |
| 6241 | ATGCTGAAGA TCAGTTGGGC GCACTAGTGG GTTACATCGA ACTGGATCTC AACAGCGGTA |
| 6301 | AGATCCTTGA GAGTTTTCGC CCCGAAGAAC GTTTTCCAAT GATGAGCACT TTTAAAGTTC |
| 6361 | TGCTATGTGG CGCGGTATTA TCCCGTATTG ACGCCGGGCA AGAGCAACTC GGTCGCCGCA |
| 6421 | TACACTATTC TCAGAATGAC TTGGTTGAGT ACTCACCAGT CACAGAAAAG CATCTTACGG |
| 6481 | ATGGCATGAC AGTAAGAGAA TTATGCAGTG CTGCCATAAC CATGAGTGAT AACACTGCGG |
| 6541 | CCAACTTACT TCTGACAACG ATCGGAGGAC CGAAGGAGCT AACCGCTTTT TTGCACAACA |
| 6601 | TGGGGGATCA TGTAACTCGC CTTGATCGTT GGGAACCGGA GCTGAATGAA GCCATACCAA |
| 6661 | ACGACGAGCG TGACACCACG ATGCCTGTAG CAATGGCAAC AACGTTGCGC AAACTATTAA |
| 6721 | CTGGCGAACT ACTTACTCTA GCTTCCCGGC AACAATTAAT AGACTGGATG GAGGCGGATA |
| 6781 | AAGTTGCAGG ACCACTTCTG CGCTCGGCCC TTCCGGCTGG CTGGTTTATT GCTGATAAAT |
| 6841 | CTGGAGCCGG TGAGCGTGGG TCTCGCGGTA TCATTGCAGC ACTGGGGCCA GATGGTAAGC |
| 6901 | CCTCCCGTAT CGTAGTTATC TACACGACGG GGAGTCAGGC AACTATGGAT GAACGAAATA |
| 6961 | GACAGATCGC TGAGATAGGT GCCTCACTGA TTAAGCATTG GTAACTGTCA GACCAAGTTT |
| 7021 | ACTCATATAT ACTTTAGATT GATTTAAAAC TTCATTTTTA ATTTAAAAGG ATCTAGGTGA |
| 7081 | AGATCCTTTT TGATAATCTC ATGACCAAAA TCCCTTAACG TGAGTTTTCG TTCCACTGTA |
| 7141 | CGTAAGACCC CCAAGCTTGT CGACTGAATG GCGAATGGCG CTTTGCCTGG TTTCCGGCAC |
| 7201 | CAGAAGCGGT GCCGGAAAGC TGGCTGGAGT GCGATCTTCC TGACGCTCGA GCGCAACGCA |
| ! | XhoI... |
| 7261 | ATTAATGTGA GTTAGCTCAC TCATTAGGCA CCCCAGGCTT TACACTTTAT GCTTCCGGCT |
| 7321 | CGTATGTTGT GTGGAATTGT GAGCGGATAA CAATTTCACA CAGGAAACAG CTATGACCAT |
| 7381 | GATTACGCCA AGCTTTGGAG CCTTTTTTTT GGAGATTTTC AAC |

TABLE 30
DNA sequence of DY3FHC87 (SEQ ID NO: 894)

| | |
|---|---|
| 1 | aatgctacta ctattagtag aattgatgcc accttttcag ctcgcgcccc aaatgaaaat |
| 61 | atagctaaac aggttattga ccatttgcga aatgtatcta atggtcaaac taaatctact |
| 121 | cgttcgcaga attgggaatc aactgttata tggaatgaaa cttccagaca ccgtacttta |
| 181 | gttgcatatt taaaacatgt tgagctacag cattatattc agcaattaag ctctaagcca |
| 241 | tccgcaaaaa tgacctctta tcaaaaggag caattaaagg tactctctaa tcctgacctg |
| 301 | ttggagtttg cttccggtct ggttcgcttt gaagctcgaa ttaaaacgcg atatttgaag |
| 361 | tctttcgggc ttcctcttaa tctttttgat gcaatccgct ttgcttctga ctataatagt |
| 421 | cagggtaaag acctgatttt tgatttatgg tcattctcgt tttctgaact gtttaaagca |
| 481 | tttgaggggg attcaatgaa tatttatgac gattccgcag tattggacgc tatccagtct |
| 541 | aaacatttta ctattacccc ctctggcaaa acttcttttg caaaagcctc tcgctatttt |
| 601 | ggtttttatc gtcgtctggt aaacgagggt tatgatagtg ttgctcttac tatgcctcgt |
| 661 | aattcctttt ggcgttatgt atctgcatta gttgaatgtg gtattcctaa atctcaactg |
| 721 | atgaatcttt ctacctgtaa taatgttgtt ccgttagttc gttttattaa cgtagatttt |
| 781 | tcttcccaac gtcctgactg gtataatgag ccagttctta aaatcgcata aggtaattca |
| 841 | caatgattaa agttgaaatt aaaccatctc aagcccaatt tactactcgt tctggtgttt |
| 901 | ctcgtcaggg caagccttat tcactgaatg agcagctttg ttacgttgat ttgggtaatg |
| 961 | aatatccggt tcttgtcaag attactcttg atgaaggtca gccagcctat gcgcctggtc |
| 1021 | tgtacaccgt tcatctgtcc tctttcaaag ttggtcagtt cggttccctt atgattgacc |
| 1081 | gtctgcgcct cgttccggct aagtaacatg gagcaggtcg cggatttcga cacaatttat |
| 1141 | caggcgatga tacaaatctc cgttgtactt tgtttcgcgc ttggtataat cgctgggggt |
| 1201 | caaagatgag tgttttagtg tattcttttg cctctttcgt tttaggttgg tgccttcgta |
| 1261 | gtggcattac gtattttacc cgtttaatgg aaacttcctc atgaaaaagt ctttagtcct |
| 1321 | caaagcctct gtagccgttg ctaccctcgt tccgatgctg tctttcgctg ctgagggtga |
| 1381 | cgatcccgca aaagcggcct ttaactccct gcaagcctca gcgaccgaat atatcggtta |
| 1441 | tgcgtgggcg atggttgttg tcattgtcgg cgcaactatc ggtatcaagc tgtttaagaa |
| 1501 | attcacctcg aaagcaagct gataaaccga tacaattaaa ggctcctttt ggagcctttt |
| 1561 | tttttggaga ttttcaacgt gaaaaaatta ttattcgcaa ttcctttagt tgttcctttc |
| 1621 | tattctcact ccgctgaaac tgttgaaagt tgtttagcaa aatcccatac agaaaattca |
| 1681 | tttactaacg tctggaaaga cgacaaaact ttagatcgtt acgctaacta tgagggctgt |
| 1741 | ctgtggaatg ctacaggcgt tgtagtttgt actggtgacg aaactcagtg ttacggtaca |
| 1801 | tgggttccta ttgggcttgc tatccctgaa aatgagggtg gtggctctga gggtggcggt |
| 1861 | tctgagggtg gcggttctga gggtggcggt actaaacctc ctgagtacgg tgatacacct |
| 1921 | attccgggct atacttatat caaccctctc gacggcactt atccgcctgg tactgagcaa |
| 1981 | aaccccgcta atcctaatcc ttctcttgag gagtctcagc ctcttaatac tttcatgttt |
| 2041 | cagaataata ggttccgaaa taggcagggg gcattaactg tttatacggg cactgttact |
| 2101 | caaggcactg accccgttaa aacttattac cagtacactc ctgtatcatc aaaagccatg |
| 2161 | tatgacgctt actggaacgg taaattcaga gactgcgctt tccattctgg ctttaatgag |
| 2221 | gatttatttg tttgtgaata tcaaggccaa tcgtctgacc tgcctcaacc tcctgtcaat |
| 2281 | gctggcggcg gctctggtgg tggttctggt ggcggctctg agggtggtgg ctctgagggt |
| 2341 | ggcggttctg agggtggcgg ctctgaggga ggcggttccg gtggtggctc tggttccggt |
| 2401 | gattttgatt atgaaaagat ggcaaacgct aataaggggg ctatgaccga aaatgccgat |
| 2461 | gaaaacgcgc tacagtctga cgctaaaggc aaacttgatt ctgtcgctac tgattacggt |
| 2521 | gctgctatcg atggtttcat tggtgacgtt tccggccttg ctaatggtaa tggtgctact |
| 2581 | ggtgattttg ctggctctaa ttcccaaatg gctcaagtcg gtgacggtga taattcacct |
| 2641 | ttaatgaata atttccgtca atatttacct tccctccctc aatcggttga atgtcgccct |
| 2701 | tttgtctttg gcgctggtaa accatatgaa ttttctattg attgtgacaa aataaactta |
| 2761 | ttccgtggtg tctttgcgtt tcttttatat gttgccacct ttatgtatgt attttctacg |
| 2821 | tttgctaaca tactgcgtaa taaggagtct taatcatgcc agttcttttg ggtattccgt |
| 2881 | tattattgcg tttcctcggt ttccttctgg taactttgtt cggctatctg cttacttttc |
| 2941 | ttaaaaaggg cttcggtaag atagctattg ctatttcatt gtttcttgct cttattattg |
| 3001 | ggcttaactc aattcttgtg ggttatctct ctgatattag cgctcaatta ccctctgact |
| 3061 | ttgttcaggg tgttcagtta attctcccgt ctaatgcgct tccctgtttt tatgttattc |
| 3121 | tctctgtaaa ggctgctatt ttcatttttg acgttaaaca aaaaatcgtt tcttatttgg |
| 3181 | attgggataa ataatatggc tgtttatttt gtaactggca aattaggctc tggaaagacg |
| 3241 | ctcgttagcg ttggtaagat tcaggataaa attgtagctg ggtgcaaaat agcaactaat |
| 3301 | cttgatttaa ggcttcaaaa cctcccgcaa gtcgggaggt tcgctaaaac gcctcgcgtt |
| 3361 | cttagaatac cggataagcc ttctatatct gatttgcttg ctattgggcg cggtaatgat |
| 3421 | tcctacgatg aaaataaaaa cggcttgctt gttctcgatg agtgcggtac ttggtttaat |
| 3481 | acccgttctt ggaatgataa ggaaagacag ccgattattg attggtttct acatgctcgt |
| 3541 | aaattaggat gggatattat ttttcttgtt caggacttat ctattgttga taaacaggcg |
| 3601 | cgttctgcat tagctgaaca tgttgtttat tgtcgtcgtc tggacagaat tactttacct |
| 3661 | tttgtcggta ctttatattc tcttattact ggctcgaaaa tgcctctgcc taaattacat |
| 3721 | gttggcgttg ttaaatatgg cgattctcaa ttaagcccta ctgttgagcg ttggctttat |
| 3781 | actggtaaga atttgtataa cgcatatgat actaaacagg ctttttctag taattatgat |
| 3841 | tccggtgttt attcttattt aacgccttat ttatcacacg gtcggtattt caaaccatta |
| 3901 | aatttaggtc agaagatgaa attaactaaa atatatttga aaaagttttc tcgcgttctt |
| 3961 | tgtcttgcga ttggatttgc atcagcattt acatatagtt atataaccca acctaagccg |
| 4021 | gaggttaaaa aggtagtctc tcagacctat gattttgata aattcactat tgactcttct |
| 4081 | cagcgtctta atctaagcta tcgctatgtt ttcaaggatt ctaagggaaa attaattaat |
| 4141 | agcgacgatt tacagaagca aggttattca ctcacatata ttgatttatg tactgtttcc |
| 4201 | attaaaaaag gtaattcaaa tgaaattgtt aaatgtaatt aattttgttt tcttgatgtt |
| 4261 | tgtttcatca tcttcttttg ctcaggtaat tgaaatgaat aattcgcctc tgcgcgattt |
| 4321 | tgtaacttgg tattcaaagc aatcaggcga atccgttatt gtttctcccg atgtaaaagg |
| 4381 | tactgttact gtatattcat ctgacgttaa acctgaaaat ctacgcaatt tctttatttc |
| 4441 | tgttttacgt gcaaataatt ttgatatggt aggttctaac ccttccataa ttcagaagta |
| 4501 | taatccaaac aatcaggatt atattgatga attgccatca tctgataatc aggaatatga |
| 4561 | tgataattcc gctccttctg gtggtttctt tgttccgcaa aatgataatg ttactcaaac |
| 4621 | ttttaaaatt aataacgttc gggcaaagga tttaatacga gttgtcgaat tgtttgtaaa |
| 4681 | gtctaatact tctaaatcct caaatgtatt atctattgac ggctctaatc tattagttgt |
| 4741 | tagtgctcct aaagatattt tagataacct tcctcaattc ctttcaactg ttgatttgcc |
| 4801 | aactgaccag atattgattg agggtttgat atttgaggtt cagcaaggtg atgctttaga |
| 4861 | tttttcattt gctgctggct ctcagcgtgg cactgttgca ggcggtgtta atactgaccg |
| 4921 | cctcacctct gttttatctt ctgctggtgg ttcgttcggt atttttaatg gcgatgtttt |
| 4981 | agggctatca gttcgcgcat taaagactaa tagccattca aaaatattgt ctgtgccacg |
| 5041 | tattcttacg ctttcaggtc agaagggttc tatctctgtt ggccagaatg tcccttttat |
| 5101 | tactggtcgt gtgactggtg aatctgccaa tgtaaataat ccatttcaga cgattgagcg |
| 5161 | tcaaaatgta ggtatttcca tgagcgtttt tcctgttgca atggctggcg gtaatattgt |
| 5221 | tctggatatt accagcaagg ccgatagttt gagttcttct actcaggcaa gtgatgttat |
| 5281 | tactaatcaa agaagtattg ctacaacggt taatttgcgt gatggacaga ctcttttact |
| 5341 | cggtggcctc actgattata aaaacacttc tcaggattct ggcgtaccgt tcctgtctaa |
| 5401 | aatcccttta atcggcctcc tgtttagctc ccgctctgat tctaacgagg aaagcacgtt |
| 5461 | atacgtgctc gtcaaagcaa ccatagtacg cgccctgtag cggcgcatta agcgcggcgg |
| 5521 | gtgtggtggt tacgcgcagc gtgaccgcta cacttgccag cgccctagcg cccgctcctt |
| 5581 | tcgctttctt cccttccttt ctcgccacgt tcgccggctt tccccgtcaa gctctaaatc |
| 5641 | gggggctccc tttagggttc cgatttagtg ctttacggca cctcgacccc aaaaaacttg |
| 5701 | atttgggtga tggttcacgt agtgggccat cgccctgata gacggttttt cgccctttga |
| 5761 | cgttggagtc cacgttcttt aatagtggac tcttgttcca aactggaaca acactcaacc |
| 5821 | ctatctcggg ctattctttt gatttataag ggattttgcc gatttcggaa ccaccatcaa |
| 5881 | acaggatttt cgcctgctgg ggcaaaccag cgtggaccgc ttgctgcaac tctctcaggg |
| 5941 | ccaggcggtg aagggcaatc agctgttgcc cgtctcactg gtgaaaagaa aaaccaccct |
| 6001 | ggatccaagc ttgcaggtgg cacttttcgg ggaaatgtgc gcggaacccc tatttgttta |
| 6061 | tttttctaaa tacattcaaa tatgtatccg ctcatgagac aataaccctg ataaatgctt |
| 6121 | caataatatt gaaaaaggaa gagtatgagt attcaacatt tccgtgtcgc ccttattccc |
| 6181 | ttttttgcgg cattttgcct tcctgttttt gctcacccag aaacgctggt gaaagtaaaa |
| 6241 | gatgctgaag atcagttggg cgcactagtg ggttacatcg aactggatct caacagcggt |
| 6301 | aagatccttg agagttttcg ccccgaagaa cgttttccaa tgatgagcac ttttaaagtt |
| 6361 | ctgctatgtg gcgcggtatt atcccgtatt gacgccgggc aagagcaact cggtcgccgc |
| 6421 | atacactatt ctcagaatga cttggttgag tactcaccag tcacagaaaa gcatcttacg |
| 6481 | gatggcatga cagtaagaga attatgcagt gctgccataa ccatgagtga taacactgcg |
| 6541 | gccaacttac ttctgacaac gatcggagga ccgaaggagc taaccgcttt tttgcacaac |
| 6601 | atgggggatc atgtaactcg ccttgatcgt tgggaaccgg agctgaatga agccatacca |
| 6661 | aacgacgagc gtgacaccac gatgcctgta gcaatggcaa caacgttgcg caaactatta |
| 6721 | actggcgaac tacttactct agcttcccgg caacaattaa tagactggat ggaggcggat |
| 6781 | aaagttgcag gaccacttct gcgctcggcc cttccggctg gctggtttat tgctgataaa |
| 6841 | tctggagccg gtgagcgtgg gtctcgcggt atcattgcag cactggggcc agatggtaag |
| 6901 | ccctcccgta tcgtagttat ctacacgacg gggagtcagg caactatgga tgaacgaaat |
| 6961 | agacagatcg ctgagatagg tgcctcactg attaagcatt ggtaactgtc agaccaagtt |
| 7021 | tactcatata tactttagat tgatttaaaa cttcattttt aatttaaaag gatctaggtg |
| 7081 | aagatccttt ttgataatct catgaccaaa atcccttaac gtgagttttc gttccactgt |
| 7141 | acgtaagacc cccaagcttg tcgactgaat ggcgaatggc gctttgcctg gtttccggca |
| 7201 | ccagaagcgg tgccggaaag ctggctggag tgcgatcttc ctgacgctcg agcgcaacgc |
| 7261 | aattaatgtg agttagctca ctcattaggc accccaggct ttacacttta tgcttccggc |
| 7321 | tcgtatgttg tgtggaattg tgagcggata acaatttcac acaggaaaca gctatgacca |
| 7381 | tgattacgcc aagctttgga gccttttttt tggagatttt caacatgaaa tacctattgc |
| 7441 | ctacggcagc cgctggattg ttattactcg cGGCCcagcc GGCCatggcc gaagttcaat |
| 7501 | tgttagagtc tggtggcggt cttgttcagc ctggtggttc tttacgtctt tcttgcgctg |
| 7561 | cttccggatt cactttctct tcgtacgcta tgtcttgggt tcgccaagct cctggtaaag |
| 7621 | gtttggagtg ggtttctgct atctctggtt ctggtggcag tacttactat gctgactccg |
| 7681 | ttaaaggtcg cttcactatc tctagagaca actctaagaa tactctctac ttgcagatga |
| 7741 | acagcttaag ggctgaggac actgcagtct actattgcgc taaagcctat cgtccttctt |
| 7801 | atcatgacat atggggtcaa ggtactatgg tcaccgtctc tagtgcctcc accaagggcc |
| 7861 | catcggtctt cccgctagca ccctcctcca agagcacctc tgggggcaca gcggccctgg |
| 7921 | gctgcctggt caaggactac ttccccgaac cggtgacggt gtcgtggaac tcaggcgccc |
| 7981 | tgaccagcgg cgtccacacc ttcccggctg tcctacagtc ctcaggactc tactccctca |
| 8041 | gcagcgtagt gaccgtgccc tccagcagct tgggcaccca gacctacatc tgcaacgtga |
| 8101 | atcacaagcc cagcaacacc aaggtggaca agaaagttga gcccaaatct tgtgcggccg |
| 8161 | cacatcatca tcaccatcac ggggccgcag aacaaaaact catctcagaa gaggatctga |
| 8221 | atggggccgc agaggctagc tctgctagtg gcgacttcga ctacgagaaa atggctaatg |
| 8281 | ccaacaaagg cgccatgact gagaacgctg acgagaatgc tttgcaaagc gatgccaagg |
| 8341 | gtaagttaga cagcgtcgcg accgactatg gcgccgccat cgacggcttt atcggcgatg |
| 8401 | tcagtggttt ggccaacggc aacggagcca ccggagactt cgcaggttcg aattctcaga |
| 8461 | tggcccaggt tggagatggg gacaacagtc cgcttatgaa caactttaga cagtaccttc |
| 8521 | cgtctcttcc gcagagtgtc gagtgccgtc cattcgtttt cggtgccggc aagccttacg |
| 8581 | agttcagcat cgactgcgat aagatcaatc ttttccgcgg cgttttcgct ttcttgctat |
| 8641 | acgtcgctac tttcatgtac gttttcagca ctttcgccaa tattttacgc aacaaagaaa |
| 8701 | gctagtgatc tcctaggaag cccgcctaat gagcgggctt tttttttctg gtatgcatcc |
| 8761 | tgaggccgat actgtcgtcg tcccctcaaa ctggcagatg cacggttacg atgcgcccat |
| 8821 | ctacaccaac gtgacctatc ccattacggt caatccgccg tttgttccca cggagaatcc |
| 8881 | gacgggttgt tactcgctca catttaatgt tgatgaaagc tggctacagg aaggccagac |
| 8941 | gcgaattatt tttgatggcg ttcctattgg ttaaaaaatg agctgattta acaaaaattt |
| 9001 | aatgcgaatt ttaacaaaat attaacgttt acaatttaaa tatttgctta tacaatcttc |
| 9061 | ctgtttttgg ggcttttctg attatcaacc ggggtacata tgattgacat gctagtttta |
| 9121 | cgattaccgt tcatcgattc tcttgtttgc tccagactct caggcaatga cctgatagcc |
| 9181 | tttgtagatc tctcaaaaat agctaccctc tccggcatta atttatcagc tagaacggtt |
| 9241 | gaatatcata ttgatggtga tttgactgtc tccggccttt ctcacccttt tgaatcttta |
| 9301 | cctacacatt actcaggcat tgcatttaaa atatatgagg gttctaaaaa tttttatcct |
| 9361 | tgcgttgaaa taaaggcttc tcccgcaaaa gtattacagg gtcataatgt ttttggtaca |
| 9421 | accgatttag ctttatgctc tgaggcttta ttgcttaatt ttgctaattc tttgccttgc |
| 9481 | ctgtatgatt tattggatgt t |

TABLE 35
DNA sequence of pMID21: 5957 bp (SEQ ID NO: 895)

| | |
|---|---|
| 1 | gacgaaaggg cctcgtgata cgcctatttt tataggttaa tgtcatgata ataatggttt |
| 61 | cttagacgtc aggtggcact tttcggggaa atgtgcgcgg aacccctatt tgtttatttt |
| 121 | tctaaataca ttcaaatatg tatccgctca tgagacaata accctgataa atgcttcaat |
| 181 | aatattgaaa aaggaagagt atgagtattc aacatttccg tgtcgccctt attccctttt |
| 241 | ttgcggcatt ttgccttcct gtttttgctc acccagaaac gctggtgaaa gtaaaagatg |
| 301 | ctgaagatca gttgggtgcc cgagtgggtt acatcgaact ggatctcaac agcggtaaga |
| 361 | tccttgagag ttttcgcccc gaagaacgtt ttccaatgat gagcactttt aaagttctgc |
| 421 | tatgtggcgc ggtattatcc cgtattgacg ccgggcaaga gcaactcggt cgccgcatac |
| 481 | actattctca gaatgacttg gttgagtact caccagtcac agaaaagcat cttacggatg |
| 541 | gcatgacagt aagagaatta tgcagtgctg ccataaccat gagtgataac actgcggcca |
| 601 | acttacttct gacaacgatc ggaggaccga aggagctaac cgcttttttg cacaacatgg |
| 661 | gggatcatgt aactcgcctt gatcgttggg aaccggagct gaatgaagcc ataccaaacg |
| 721 | acgagcgtga caccacgatg cctgtagcaa tggcaacaac gttgcgcaaa ctattaactg |
| 781 | gcgaactact tactctagct tcccggcaac aattaataga ctggatggag gcggataaag |
| 841 | ttgcaggacc acttctgcgc tcggcccttc cggctggctg gtttattgct gataaatctg |
| 901 | gagccggtga gcgtgggtct cgcggtatca ttgcagcact ggggccagat ggtaagccct |
| 961 | cccgtatcgt agttatctac acgacgggga gtcaggcaac tatggatgaa cgaaatagac |
| 1021 | agatcgctga gataggtgcc tcactgatta agcattggta actgtcagac caagtttact |
| 1081 | catatatact ttagattgat ttaaaacttc atttttaatt taaaaggatc taggtgaaga |
| 1141 | tcctttttga taatctcatg accaaaatcc cttaacgtga gttttcgttc cactgagcgt |
| 1201 | cagaccccgt agaaaagatc aaaggatctt cttgagatcc tttttttctg cgcgtaatct |
| 1261 | gctgcttgca aacaaaaaaa ccaccgctac cagcggtggt ttgtttgccg gatcaagagc |
| 1321 | taccaactct ttttccgaag gtaactggct tcagcagagc gcagatacca aatactgttc |
| 1381 | ttctagtgta gccgtagtta ggccaccact tcaagaactc tgtagcaccg cctacatacc |
| 1441 | tcgctctgct aatcctgtta ccagtggctg ctgccagtgg cgataagtcg tgtcttaccg |
| 1501 | ggttggactc aagacgatag ttaccggata aggcgcagcg gtcgggctga acggggggtt |
| 1561 | cgtgcataca gcccagcttg gagcgaacga cctacaccga actgagatac ctacagcgtg |
| 1621 | agctatgaga aagcgccacg cttcccgaag ggagaaaggc ggacaggtat ccggtaagcg |
| 1681 | gcagggtcgg aacaggagag cgcacgaggg agcttccagg gggaaacgcc tggtatcttt |
| 1741 | atagtcctgt cgggtttcgc cacctctgac ttgagcgtcg atttttgtga tgctcgtcag |
| 1801 | gggggcggag cctatggaaa aacgccagca acgcggcctt tttacggttc ctggcctttt |
| 1861 | gctggccttt tgctcacatg ttctttcctg cgttatcccc tgattctgtg gataaccgta |
| 1921 | ttaccgcctt tgagtgagct gataccgctc gccgcagccg aacgaccgag cgcagcgagt |
| 1981 | cagtgagcga ggaagcggaa gagcgcccaa tacgcaaacc gcctctcccc gcgcgttggc |
| 2041 | cgattcatta atgcagctgg cacgacaggt ttcccgactg gaaagcgggc agtgagcgca |
| 2101 | acgcaattaa tgtgagttag ctcactcatt aggcacccca ggctttacac tttatgcttc |
| 2161 | cggctcgtat gttgtgtgga attgtgagcg gataacaatt tcacacagga aacagctatg |
| 2221 | accatgatta cgccaagctt tggagccttt tttttggaga ttttcaacgt gaaaaaatta |
| 2281 | ttattcgcaa ttcctttagt tgttcctttc tattctcaca gtgcacaggt ccaactgcag |
| 2341 | gagctcgaga tcaaacgtgg aactgtggct gcaccatctg tcttcatctt cccgccatct |
| 2401 | gatgagcagt tgaaatctgg aactgcctct gttgtgtgcc tgctgaataa cttctatccc |
| 2461 | agagaggcca aagtacagtg gaaggtggat aacgccctcc aatcgggtaa ctcccaggag |
| 2521 | agtgtcacag agcaggacag caaggacagc acctacagcc tcagcagcac cctgacgctg |
| 2581 | agcaaagcag actacgagaa acacaaagtc tacgcctgcg aagtcaccca tcagggcctg |
| 2641 | agttcaccgg tgacaaagag cttcaacagg ggagagtgtt aataaggcgc gcctaaccat |
| 2701 | ctatttcaag gaacagtctt aatgaaaaag cttttattca tgatcccgtt agttgtaccg |
| 2761 | ttcgtggccc agccggcctc tgctgaagtt caattgttag agtctggtgg cggtcttgtt |
| 2821 | cagcctggtg gttctttacg tctttcttgc gctgcttccg gagcttcaga tctgtttgcc |
| 2881 | tttttgtggg gtggtgcaga tcgcgttacg gagatcgacc gactgcttga gcaaaagcca |
| 2941 | cgcttaactg ctgatcaggc atgggatgtt attcgccaaa ccagtcgtca ggatcttaac |
| 3001 | ctgaggcttt ttttacctac tctgcaagca gcgacatctg gtttgacaca gagcgatccg |
| 3061 | cgtcgtcagt tggtagaaac attaacacgt tgggatggca tcaatttgct taatgatgat |
| 3121 | ggtaaaacct ggcagcagcc aggctctgcc atcctgaacg tttggctgac cagtatgttg |
| 3181 | aagcgtaccg tagtggctgc cgtacctatg ccatttgata agtggtacag cgccagtggc |
| 3241 | tacgaaacaa cccaggacgg cccaactggt tcgctgaata taagtgttgg agcaaaaatt |
| 3301 | ttgtatgagg cggtgcaggg agacaaatca ccaatcccac aggcggttga tctgtttgct |
| 3361 | gggaaaccac agcaggaggt tgtgttggct gcgctggaag atacctggga gactctttcc |
| 3421 | aaacgctatg gcaataatgt gagtaactgg aaaacaccgg caatggcctt aacgttccgg |
| 3481 | gcaaataatt tctttggtgt accgcaggcc gcagcggaag aaacgcgtca tcaggcggag |
| 3541 | tatcaaaacc gtggaacaga aaacgatatg attgttttct caccaacgac aagcgatcgt |
| 3601 | cctgtgcttg cctgggatgt ggtcgcaccc ggtcagagtg ggtttattgc tcccgatgga |
| 3661 | acagttgata agcactatga agatcagctg aaaatgtacg aaaattttgg ccgtaagtcg |
| 3721 | ctctggttaa cgaagcagga tgtggaggcg cataaggagt tctagagaca actctaagaa |
| 3781 | tactctctac ttgcagatga acagcttaag tctgagcatt cggtccgggc aacattctcc |
| 3841 | aaactgacca gacgacacaa acggcttacg ctaaatcccg cgcatgggat ggtaaagagg |
| 3901 | tggcgtcttt gctggcctgg actcatcaga tgaaggccaa aaattggcag gagtggacac |
| 3961 | agcaggcagc gaaacaagca ctgaccatca actggtacta tgctgatgta aacggcaata |
| 4021 | ttggttatgt tcatactggt gcttatccag atcgtcaatc aggccatgat ccgcgattac |
| 4081 | ccgttcctgg tacgggaaaa tgggactgga aagggctatt gccttttgaa atgaacccta |
| 4141 | aggtgtataa cccccagcag ctagccatat tctctcggtc accgtctcaa gcgcctccac |
| 4201 | caagggccca tcggtcttcc cgctagcacc ctcctccaag agcacctctg ggggcacagc |
| 4261 | ggccctgggc tgcctggtca aggactactt ccccgaaccg gtgacggtgt cgtggaactc |
| 4321 | aggcgccctg accagcggcg tccacacctt cccggctgtc ctacagtcta gcggactcta |
| 4381 | ctccctcagc agcgtagtga ccgtgccctc ttctagcttg ggcacccaga cctacatctg |
| 4441 | caacgtgaat cacaagccca gcaacaccaa ggtggacaag aaagttgagc ccaaatcttg |
| 4501 | tgcggccgca catcatcatc accatcacgg ggccgcagaa caaaaactca tctcagaaga |
| 4561 | ggatctgaat ggggccgcag aggctagttc tgctagtaac gcgtcttccg gtgattttga |
| 4621 | ttatgaaaag atggcaaacg ctaataaggg ggctatgacc gaaaatgccg atgaaaacgc |
| 4681 | gctacagtct gacgctaaag gcaaacttga ttctgtcgct actgattacg gtgctgctat |
| 4741 | cgatggtttc attggtgacg tttccggcct tgctaatggt aatggtgcta ctggtgattt |
| 4801 | tgctggctct aattcccaaa tggctcaagt cggtgacggt gataattcac ctttaatgaa |
| 4861 | taatttccgt caatatttac cttccctccc tcaatcggtt gaatgtcgcc cttttgtctt |
| 4921 | tggcgctggt aaaccatatg aattttctat tgattgtgac aaaataaact tattccgtgg |
| 4981 | tgtctttgcg tttcttttat atgttgccac ctttatgtat gtattttcta cgtttgctaa |
| 5041 | catactgcgt aataaggagt cttaatgaaa cgcgtgatga gaattcactg gccgtcgttt |
| 5101 | tacaacgtcg tgactgggaa aaccctggcg ttacccaact taatcgcctt gcagcacatc |
| 5161 | cccctttcgc cagctggcgt aatagcgaag aggcccgcac cgatcgccct tcccaacagt |
| 5221 | tgcgcagcct gaatggcgaa tggcgcctga tgcggtattt tctccttacg catctgtgcg |
| 5281 | gtatttcaca ccgcatacgt caaagcaacc atagtacgcg ccctgtagcg gcgcattaag |
| 5341 | cgcggcgggt gtggtggtta cgcgcagcgt gaccgctaca cttgccagcg ccttagcgcc |
| 5401 | cgctcctttc gctttcttcc cttcctttct cgccacgttc gccggctttc cccgtcaagc |
| 5461 | tctaaatcgg gggctccctt tagggttccg atttagtgct ttacggcacc tcgaccccaa |
| 5521 | aaaacttgat ttgggtgatg gttcacgtag tgggccatcg ccctgataga cggtttttcg |
| 5581 | ccctttgacg ttggagtcca cgttctttaa tagtggactc ttgttccaaa ctggaacaac |
| 5641 | actcaactct atctcgggct attcttttga tttataaggg attttgccga tttcggtcta |
| 5701 | ttggttaaaa aatgagctga tttaacaaaa atttaacgcg aattttaaca aaatattaac |
| 5761 | gtttacaatt ttatggtgca gtctcagtac aatctgctct gatgccgcat agttaagcca |
| 5821 | gccccgacac ccgccaacac ccgctgacgc gccctgacgg gcttgtctgc tcccggcatc |
| 5881 | cgcttacaga caagctgtga ccgtctccgg gagctgcatg tgtcagaggt tttcaccgtc |
| 5941 | atcaccgaaa cgcgcga |

TABLE 40
pLCSK23 (SEQ ID NO: 896)

| | |
|---|---|
| 1 | GACGAAAGGG CCTGCTCTGC CAGTGTTACA ACCAATTAAC CAATTCTGAT TAGAAAAACT |
| 61 | CATCGAGCAT CAAATGAAAC TGCAATTTAT TCATATCAGG ATTATCAATA CCATATTTTT |
| 121 | GAAAAAGCCG TTTCTGTAAT GAAGGAGAAA ACTCACCGAG GCAGTTCCAT AGGATGGCAA |
| 181 | GATCCTGGTA TCGGTCTGCG ATTCCGACTC GTCCAACATC AATACAACCT ATTAATTTCC |
| 241 | CCTCGTCAAA AATAAGGTTA TCAAGTGAGA AATCACCATG AGTGACGACT GAATCCGGTG |
| 301 | AGAATGGCAA AAGCTTATGC ATTTCTTTCC AGACTTGTTC AACAGGCCAG CCATTACGCT |
| 361 | CGTCATCAAA ATCACTCGCA TCAACCAAAC CGTTATTCAT TCGTGATTGC GCCTGAGCGA |
| 421 | GACGAAATAC GCGATCGCTG TTAAAAGGAC AATTACAAAC AGGAATTGAA TGCAACCGGC |
| 481 | GCAGGAACAC TGCCAGCGCA TCAACAATAT TTTCACCTGA ATCAGGATAT TCTTCTAATA |
| 541 | CCTGGAATGC TGTTTTCCCG GGGATCGCAG TGGTGAGTAA CCATGCATCA TCAGGAGTAC |
| 601 | GGATAAAATG CTTGATGGTC GGAAGAGGCA TAAATTCCGT CAGCCAGTTT AGTCTGACCA |
| 661 | TCTCATCTGT AACATCATTG GCAACGCTAC CTTTGCCATG TTTCAGAAAC AACTCTGGCG |
| 721 | CATCGGGCTT CCCATACAAT CGATAGATTG TCGCACCTGA TTGCCCGACA TTATCGCGAG |
| 781 | CCCATTTATA CCCATATAAA TCAGCATCCA TGTTGGAATT TAATCGCGGC CTCGAGCAAG |
| 841 | ACGTTTCCCG TTGAATATGG CTCATAACAC CCCTTGTATT ACTGTTTATG TAAGCAGACA |
| 901 | GTTTTATTGT TCATGATGAT ATATTTTTAT CTTGTGCAAT GTAACATCAG AGATTTTGAG |
| 961 | ACACAACGTG GCTTTCCCCC CCCCCCCCTG CAGGTCTCGG GCTATTCCTG TCAGACCAAG |
| 1021 | TTTACTCATA TATACTTTAG ATTGATTTAA AACTTCATTT TTAATTTAAA AGGATCTAGG |
| 1081 | TGAAGATCCT TTTTGATAAT CTCATGACCA AAATCCCTTA ACGTGAGTTT TCGTTCCACT |
| 1141 | GAGCGTCAGA CCCCGTAGAA AAGATCAAAG GATCTTCTTG AGATCCTTTT TTTCTGCGCG |
| 1201 | TAATCTGCTG CTTGCAAACA AAAAAACCAC CGCTACCAGC GGTGGTTTGT TTGCCGGATC |
| 1261 | AAGAGCTACC AACTCTTTTT CCGAAGGTAA CTGGCTTCAG CAGAGCGCAG ATACCAAATA |
| 1321 | CTGTTCTTCT AGTGTAGCCG TAGTTAGGCC ACCACTTCAA GAACTCTGTA GCACCGCCTA |
| 1381 | CATACCTCGC TCTGCTAATC CTGTTACCAG TGGCTGCTGC CAGTGGCGAT AAGTCGTGTC |
| 1441 | TTACCGGGTT GGACTCAAGA CGATAGTTAC CGGATAAGGC GCAGCGGTCG GGCTGAACGG |
| 1501 | GGGGTTCGTG CATACAGCCC AGCTTGGAGC GAACGACCTA CACCGAACTG AGATACCTAC |
| 1561 | AGCGTGAGCT ATGAGAAAGC GCCACGCTTC CCGAAGGGAG AAAGGCGGAC AGGTATCCGG |
| 1621 | TAAGCGGCAG GGTCGGAACA GGAGAGCGCA CGAGGGAGCT TCCAGGGGGA AACGCCTGGT |
| 1681 | ATCTTTATAG TCCTGTCGGG TTTCGCCACC TCTGACTTGA GCGTCGATTT TTGTGATGCT |
| 1741 | CGTCAGGGGG GCGGAGCCTA TGGAAAAACG CCAGCAACGC GGCCTTTTTA CGGTTCCTGG |
| 1801 | CCTTTTGCTG GCCTTTTGCT CACATGTTCT TTCCTGCGTT ATCCCCTGAT TCTGTGGATA |
| 1861 | ACCGTATTAC CGCCTTTGAG TGAGCTGATA CCGCTCGCCG CAGCCGAACG ACCGAGCGCA |
| 1921 | GCGAGTCAGT GAGCGAGGAA GCGGAAGAGC GCCCAATACG CAAACCGCCT CTCCCCGCGC |
| 1981 | GTTGGCCGAT TCATTAATGC AGCTGGCACG ACAGGTTTCC CGACTGGAAA GCGGGCAGTG |
| 2041 | AGCGCAACGC AATTAATGTG AGTTAGCTCA CTCATTAGGC ACCCCAGGCT TTACACTTTA |
| 2101 | TGCTTCCGGC TCGTATGTTG TGTGGAATTG TGAGCGGATA ACAATTTCAC ACAGGAAACA |
| 2161 | GCTATGACCA TGATTACGCC AAGCTTTGGA GCCTTTTTTT TGGAGATTTT CAACATGAAG |
| 2221 | AAGCTCCTCT TTGCTATCCC GCTCGTCGTT CCTTTTGTGG CCCAGCCGGC CATGGCCGAC |
| 2281 | ATCCAGATGA CCCAGTCTCC ATCCTCCCTG TCTGCATCTG TAGGAGACAG AGTCACCATC |
| 2341 | ACTTGCCGGG CAAGTCAGAG CATTAGCAGC TATTTAAATT GGTATCAGCA GAAACCAGGG |
| 2401 | AAAGCCCCTA AGCTCCTGAT CTATGCTGCA TCCAGTTTGC AAAGTGGGGT CCCATCAAGG |
| 2461 | TTCAGTGGCA GTGGATCTGG GACAGATTTC ACTCTCACCA TCAGCAGTCT GCAACCTGAA |
| 2521 | GATTTTGCAA CTTACTACTG TCAACAGAGT TACAGTACCC CTTTCACTTT CGGCCCTGGG |
| 2581 | ACCAAAGTGG ATATCAAACG TGGtACcGTG GCTGCACCAT CTGTCTTCAT CTTCCCGCCA |
| 2641 | TCTGATGAGC AGTTGAAATC TGGAACTGCC TCTGTTGTGT GCCTGCTGAA TAACTTCTAT |
| 2701 | CCCAGAGAGG CCAAAGTACA GTGGAAGGTG GATAACGCCC TCCAATCGGG TAACTCCCAG |
| 2761 | GAGAGTGTCA CAGAGCAGGA CAGCAAGGAC AGCACCTACA GCCTCAGCAG CACCCTGACG |
| 2821 | CTGAGCAAAG CAGACTACGA GAAACACAAA GTCTACGCCT GCGAAGTCAC CCATCAGGGC |
| 2881 | CTGAGTTCAC CGGTGACAAA GAGCTTCAAC AGGGGAGAGT GTGCGGCCGC TGGTAAGCCT |
| 2941 | ATCCCTAACC CTCTCCTCGG TCTCGATTCT ACGTGATAAC TTCACCGGTC AACGCGTGAT |
| 3001 | GAGAATTCAC TGGCCGTCGT TTTACAACGT CGTGACTGGG AAAACCCTGG CGTTACCCAA |
| 3061 | CTTAATCGCC TTGCAGCACA TCCCCCTTTC GCCAGCTGGC GTAATAGCGA AGAGGCCCGC |
| 3121 | ACCGATCGCC CTTCCCAACA GTTGCGCAGC CTGAATGGCG AATGGCGCCT GATGCGGTAT |
| 3181 | TTTCTCCTTA CGCATCTGTG CGGTATTTCA CACCGCATAC GTCAAAGCAA CCATAGTCTC |
| 3241 | AGTACAATCT GCTCTGATGC CGCATAGTTA AGCCAGCCCC GACACCCGCC AACACCCGCT |
| 3301 | GACGCGCCCT GACAGGCTTG TCTGCTCCCG GCATCCGCTT ACAGACAAGC TGTGACCGTC |
| 3361 | TCCGGGAGCT GCATGTGTCA GAGGTTTTCA CCGTCATCAC CGAAACGCGC GA |

### REFERENCES

The contents of all cited references including literature references, issued patents, published or non-published patent applications cited throughout this application as well as those listed below are hereby expressly incorporated by reference in their entireties. In case of conflict, the present application, including any definitions herein, will control.
U.S. Published Application 2005-0119455A1
Sidhu et al., J Mol Biol. 2004 338:299-310.

### EQUIVALENTS

A number of embodiments of the invention have been described. Nevertheless, it will be understood that various modifications may be made without departing from the spirit and scope of the invention. Accordingly, other embodiments are within the scope of the following claims.

1.-19. (canceled)

20. A method of diversifying a library, the method comprising mutagenizing a library comprising a HC CDR3 that is 3, 4, or 5 amino acids in length, wherein the CDR3 comprises amino acids from a JH region or from a D region joined to the FR4 portion of a JH region.

21. The method of claim 20, wherein the HC CDR3 is from a D region joined to the FR4 portion of a JH region and comprises a trimer, a tetramer, or a pentamer, wherein the trimer, tetramer, or pentamer does not comprise a cysteine residue.

22. The method of claim 20, wherein the HC CDR3 is from a D region joined to the FR4 portion of a JH region and comprises a trimer, a tetramer, or a pentamer, wherein the trimer, tetramer, or pentamer does not comprise a stop codon.

23. The method of claim 20, wherein the D region comprises a TAG codon and the TAG codon is replaced by a codon selected from the group consisting of TCG, TTG, TGG, CAG, AAG, TAT, and GAG.

24. The method of claim 20, wherein the D region comprises a TAA codon and the TAA codon is replaced by a codon selected from the group consisting of TCA, TTA, CAA, AAA, TAT, and GAA.

25. The method of claim 20, wherein the D region comprises a TGA codon and the TGA codon is replaced by a codon selected from the group consisting of TGG, TCA, TTA, AGA, and GGA.

26. The method of claim 20, wherein the HC CDR3 is enriched in Tyr (Y) and Ser(S).

27. The method of claim 20, wherein the D region is selected from the group consisting of D2-2 (RF 2), D2-8 (RF 2), D2-15 (RF 2), D2-21 (RF 2), D3-16 (RF 2), D3-22 (RF 2), D3-3 (RF-2), D3-9 (RF 2), D3-10 (RF 2), D1-26 (RF 3), D4-11 (RF 2), D4-4 (RF 2), D5-5 (RF 3), D5-12 (RF 3), D5-18 (RF 3), D6-6 (RF1), D6-13 (RF 1), and D6-19 (RF 1).

28. The method of claim 20, wherein the D region comprises one or more cysteine (Cys) residues and the one or more Cys residues are held constant.

29. The method of claim 20, wherein the HC CDR3 comprises one or more filling codons between FR3 and the D region and each filling codon is individually NNK, TMY, TMT, or TMC.

30. The method of claim 20, wherein the HC CDR3 comprises one or more filling codons between the D region and JH and each filling codon is individually NNK, TMY, TMT, or TMC.

31. The method of claim 20, wherein the library further comprises a HC CDR1, HC CDR2, or a light chain and comprises diversity in the HC CDR1, HC CDR2, or light chain.

32. The method of claim 20, wherein the mutagenizing comprises error-prone PCR.

33. The method of claim 20, wherein the mutagenizing comprises wobbling.

34. The method of claim 20, wherein the mutagenizing comprises dobbling.

35. The method of claim 20, wherein the mutagenizing introduces on average about 1 to about 10 mutations per HC CDR3.


SEQUENCE LISTING (943 sequences)

SEQ ID NO: 1 - protein, 15 aa, Homo sapiens
YFDYWGQGTLVTVSS

SEQ ID NO: 2 - protein, 15 aa, Homo sapiens
AFDIWGQGTMVTVSS

SEQ ID NO: 3 - protein, 20 aa, Homo sapiens
YYYYYGMDVWGQGTTVTVSS

SEQ ID NO: 4 - protein, 107 aa, synthetic construct
  REGION 1..107: note = Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 5 - protein, 107 aa, synthetic construct
  REGION 1..107: note = Synthetic polypeptide
DIQMTQSPSSLSASVGDRVTITCRASQDISSYLNWYQQKPGKAPKLLIYAASSLQSGVPSRFSGSGSGTDFTLTISSLQPEDFATYYCQQSYSTPFTFGPGTKVDIK

SEQ ID NO: 6 - protein, 107 aa, synthetic construct
  REGION 1..107: note = Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQRYSTPFTFGPGTKVDIK

SEQ ID NO: 7 - protein, 107 aa, synthetic construct
  REGION 1..107: note = Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQEYSTPFTFGPGTKVDIK

SEQ ID NO: 8 - protein, 107 aa, synthetic construct
  REGION 1..107: note = Synthetic polypeptide
DIQMTQSPSSLSASVGDRVTITCRASQSISRYLNWYQQKPGKAPKLLIYAASSLQSGVPSRFSGSGSGTDFTLTISSLQPEDFATYYCQQSYSTPFTFGPGTKVDIK

SEQ ID NO: 9 - protein, 107 aa, synthetic construct
  REGION 1..107: note = Synthetic polypeptide
DIQMTQSPSSLSASVGDRVTITCRASQSISEYLNWYQQKPGKAPKLLIYAASSLQSGVPSRFSGSGSGTDFTLTISSLQPEDFATYYCQQSYRTPFTFGPGTKVDIK

SEQ ID NO: 10 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 11 - protein, 107 aa, synthetic construct
  REGION 1..107: note = Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQRSNWPLTFGGGTKVEIK

SEQ ID NO: 12 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 13 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRGNWPLTFGGGTKVEIK

SEQ ID NO: 14 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRYNWPLTFGGGTKVEIK

SEQ ID NO: 15 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRENWPLTFGGGTKVEIK

SEQ ID NO: 16 - protein, 107 aa, synthetic construct
  REGION 1..107: note = Synthetic polypeptide
EIVLTQSPATLSLSPGERATLSCRASQSVSSFLAWYQQKPGQAPRLLIYDASNRATGIPARFSGSGSGTDFTLTISSLEPEDFAVYYCQQRSNWPLTFGGGTKVEIK

SEQ ID NO: 17 - protein, 107 aa, synthetic construct
  REGION 1..107: note = Synthetic polypeptide
EIVLTQSPATLSLSPGERATLSCRASQSVSSDLAWYQQKPGQAPRLLIYDASNRATGIPARFSGSGSGTDFTLTISSLEPEDFAVYYCQQRSNWPLTFGGGTKVEIK

SEQ ID NO: 18 - protein, 107 aa, synthetic construct
  REGION 1..107: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSGWPLTFGGGTKVEIK

SEQ ID NO: 19 - protein, 108 aa, synthetic construct
  REGION 1..108: note = Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 20 - protein, 108 aa, synthetic construct
  REGION 1..108: note = Synthetic polypeptide
EIVLTQSPGTLSLSPGERATLSCRASQSVSRSYLAWYQQKPGQAPRLLIYGASSRATGIPDRFSGSGSGTDFTLTISRLEPEDFAVYYCQQYGSSPFTFGPGTKVDIK

SEQ ID NO: 21 - protein, 108 aa, synthetic construct
  REGION 1..108: note = Synthetic polypeptide
EIVLTQSPGTLSLSPGERATLSCRASQSVSSRYLAWYQQKPGQAPRLLIYGASSRATGIPDRFSGSGSGTDFTLTISRLEPEDFAVYYCQQYGSSPFTFGPGTKVDIK

SEQ ID NO: 22 - protein, 108 aa, synthetic construct
  REGION 1..108: note = Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQQYGSSPFTFGPGTKVDIK

SEQ ID NO: 23 - protein, 108 aa, synthetic construct
  REGION 1..108: note = Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYESSPFTFGPGTKVDIK

SEQ ID NO: 24 - protein, 108 aa, synthetic construct
  REGION 1..108: note = Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYRSSPFTFGPGTKVDIK

SEQ ID NO: 25 - protein, 108 aa, synthetic construct
  REGION 1..108: note = Synthetic polypeptide
EIVLTQSPGTLSLSPGERATLSCRASQSVDSSYLAWYQQKPGQAPRLLIYGASSRATGIPDRFSGSGSGTDFTLTISRLEPEDFAVYYCQQYESSPFTFGPGTKVDIK

SEQ ID NO: 26 - protein, 108 aa, synthetic construct
  REGION 1..108: note = Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFTFGPGTKVDIK

SEQ ID NO: 27 - protein, 107 aa, synthetic construct
  REGION 1..107: note = Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 28 - protein, 107 aa, synthetic construct
  REGION 1..107: note = Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPITFGQGTRLEIK

SEQ ID NO: 29 - protein, 4 aa, Mus sp.
NWDY

SEQ ID NO: 30 - protein, 8 aa, synthetic construct
  REGION 1..8: note = Synthetic peptide
TAVYYCAK

SEQ ID NO: 31 - protein, 22 aa, synthetic construct
  REGION 1..22: note = Synthetic peptide
TAVYYCAKFQHWGQGTLVTVSS

SEQ ID NO: 32 - protein, 22 aa, synthetic construct
  REGION 1..22: note = Synthetic peptide
TAVYYCAKFDLWGRGTLVTVSS

SEQ ID NO: 33 - protein, 22 aa, synthetic construct
  REGION 1..22: note = Synthetic peptide
TAVYYCAKFDIWGQGTMVTVSS

SEQ ID NO: 34 - protein, 22 aa, synthetic construct
  REGION 1..22: note = Synthetic peptide
TAVYYCAKFDYWGQGTLVTVSS

SEQ ID NO: 35 - protein, 22 aa, synthetic construct
  REGION 1..22: note = Synthetic peptide
TAVYYCAKFDPWGQGTLVTVSS

SEQ ID NO: 36 - protein, 22 aa, synthetic construct
  REGION 1..22: note = Synthetic peptide
TAVYYCAKMDVWGQGTTVTVSS

SEQ ID NO: 37 - protein, 22 aa, synthetic construct
  REGION 1..22: note = Synthetic peptide
TAVYYCAKGTTWGQGTLVTVSS

SEQ ID NO: 38 - protein, 22 aa, synthetic construct
  REGION 1..22: note = Synthetic peptide
TAVYYCAKTTGWGQGTLVTVSS

SEQ ID NO: 39 - protein, 22 aa, synthetic construct
  REGION 1..22: note = Synthetic peptide
TAVYYCAKIFGWGRGTLVTVSS

SEQ ID NO: 40 - protein, 23 aa, synthetic construct
  REGION 1..23: note = Synthetic peptide
TAVYYCAKYFQHWGQGTLVTVSS

SEQ ID NO: 41 - protein, 23 aa, synthetic construct
  REGION 1..23: note = Synthetic peptide
TAVYYCAKYFDLWGRGTLVTVSS

SEQ ID NO: 42 - protein, 23 aa, synthetic construct
  REGION 1..23: note = Synthetic peptide
TAVYYCAKAFDIWGQGTMVTVSS

SEQ ID NO: 43 - protein, 23 aa, synthetic construct
  REGION 1..23: note = Synthetic peptide
TAVYYCAKYFDYWGQGTLVTVSS

SEQ ID NO: 44 - protein, 23 aa, synthetic construct
  REGION 1..23: note = Synthetic peptide
TAVYYCAKWFDPWGQGTLVTVSS

SEQ ID NO: 45 - protein, 23 aa, synthetic construct
  REGION 1..23: note = Synthetic peptide
TAVYYCAKGMDVWGQGTTVTVSS

SEQ ID NO: 46 - protein, 23 aa, synthetic construct
  REGION 1..23: note = Synthetic peptide
TAVYYCAKLLWFWGRGTLVTVSS

SEQ ID NO: 47 - protein, 24 aa, synthetic construct
  REGION 1..24: note = Synthetic peptide
TAVYYCAKEYFQHWGQGTLVTVSS

SEQ ID NO: 48 - protein, 24 aa, synthetic construct
  REGION 1..24: note = Synthetic peptide
TAVYYCAKWYFDLWGRGTLVTVSS

SEQ ID NO: 49 - protein, 24 aa, synthetic construct
  REGION 1..24: note = Synthetic peptide
TAVYYCAKYAFDIWGQGTMVTVSS

SEQ ID NO: 50 - protein, 24 aa, synthetic construct
  REGION 1..24: note = Synthetic peptide
TAVYYCAKSYFDYWGQGTLVTVSS

SEQ ID NO: 51 - protein, 24 aa, synthetic construct
  REGION 1..24: note = Synthetic peptide
TAVYYCAKNWFDPWGQGTLVTVSS

SEQ ID NO: 52 - protein, 24 aa, synthetic construct
  REGION 1..24: note = Synthetic peptide
TAVYYCAKYGMDVWGQGTTVTVSS

SEQ ID NO: 53 - protein, 24 aa, synthetic construct
  REGION 1..24: note = Synthetic peptide
TAVYYCAKDIVLMWGRGTLVTVSS

SEQ ID NO: 54 - protein, 27 aa, synthetic construct
  REGION 1..27: note = Synthetic peptide
AEDTAVYYCAKDIVLMWGQGTTVTVSS

SEQ ID NO: 55 - DNA, 81 bp, synthetic construct
  misc_feature 1..81: note = Synthetic oligonucleotide
  source 1..81: mol_type = other DNA
gctgaggatactgcagtttattactgcgctaagnnnnnnnnnnnnnnntggggccagggtactacggtcaccgtctccagt

SEQ ID NO: 56 - protein, 42 aa, synthetic construct
  REGION 1..42: note = Synthetic polypeptide
  VARIANT 28: note = May be R or K
SRDNSKNTLYLQMNSLRAEDTAVYYCAXXXXWGQGTTVTVSS

SEQ ID NO: 57 - DNA, 135 bp, synthetic construct
  misc_feature 1..135: note = Synthetic polynucleotide
  source 1..135: mol_type = other DNA
  CDS 10..135
ttcactatctctagagacaactctaagaatactctctacttgcagatgaacagcttaagggctgaggatactgcagtttattactgcgctargnnknnknnktggggccagggtactacggtcaccgtctccagt

SEQ ID NO: 58 - DNA, 48 bp, synthetic construct
  misc_feature 1..48: note = Synthetic oligonucleotide
  source 1..48: mol_type = other DNA
tgcagtttattactgcgctargnnknnknnktggggccagggtactac

SEQ ID NO: 59 - DNA, 33 bp, synthetic construct
  misc_feature 1..33: note = Synthetic oligonucleotide
  source 1..33: mol_type = other DNA
tggggccagggtactacggtcaccgtctccagt

SEQ ID NO: 60 - protein, 43 aa, synthetic construct
  REGION 1..43: note = Synthetic polypeptide
  VARIANT 28: note = R or K
SRDNSKNTLYLQMNSLRAEDTAVYYCAXXXXXWGQGTTVTVSS

SEQ ID NO: 61 - DNA, 138 bp, synthetic construct
  misc_feature 1..138: note = Synthetic polynucleotide
  source 1..138: mol_type = other DNA
  CDS 10..138
ttcactatctctagagacaactctaagaatactctctacttgcagatgaacagcttaagggctgaggatactgcagtttattactgcgctargnnknnknnknnktggggccagggtactacggtcaccgtctccagt

SEQ ID NO: 62 - DNA, 50 bp, synthetic construct
  misc_feature 1..50: note = Synthetic oligonucleotide
  source 1..50: mol_type = other DNA
gcagtttattactgcgctargnnknnknnknnktggggccagggtactac

SEQ ID NO: 63 - protein, 44 aa, synthetic construct
  REGION 1..44: note = Synthetic polypeptide
  VARIANT 28: note = R or K
SRDNSKNTLYLQMNSLRAEDTAVYYCAXXXXXXWGQGTTVTVSS

SEQ ID NO: 64 - DNA, 141 bp, synthetic construct
  misc_feature 1..141: note = Synthetic polynucleotide
  source 1..141: mol_type = other DNA
  CDS 10..141
ttcactatctctagagacaactctaagaatactctctacttgcagatgaacagcttaagggctgaggatactgcagtttattactgcgctargnnknnknnknnknnktggggccagggtactacggtcaccgtctccagt

SEQ ID NO: 65 - DNA, 53 bp, synthetic construct
  misc_feature 1..53: note = Synthetic oligonucleotide
  source 1..53: mol_type = other DNA
gcagtttattactgcgctargnnknnknnknnknnktggggccagggtactac

SEQ ID NO: 66 - protein, 17 aa, Homo sapiens
AEYFQHWGQGTLVTVSS

SEQ ID NO: 67 - protein, 17 aa, Homo sapiens
YWYFDLWGRGTLVTVSS

SEQ ID NO: 68 - protein, 16 aa, Homo sapiens
NWFDPWGQGTLVTVSS

SEQ ID NO: 69 - protein, 39 aa, synthetic construct
  REGION 1..39: note = Synthetic polypeptide
YYCAXXXGYCSXXSCYTXXYSYAEYFQHWGQGTLVTVSS

SEQ ID NO: 70 - protein, 10 aa, Homo sapiens
GYCSSTSCYT

SEQ ID NO: 71 - protein, 62 aa, synthetic construct
  REGION 1..62: note = Synthetic polypeptide
  VARIANT 28: note = R or K
SRDNSKNTLYLQMNSLRAEDTAVYYCAXXXGYCSXXSCYTXXYSYAEYFQHWGQGTLVTVSS

SEQ ID NO: 72 - DNA, 195 bp, synthetic construct
  misc_feature 1..195: note = Synthetic polynucleotide
  source 1..195: mol_type = other DNA
  CDS 10..195
ttcactatctctagagacaactctaagaatactctctacttgcagatgaacagcttaagggctgaggatactgcagtttattactgcgctargnnknnkggttattgttccnnknnktcttgctatactnnknnktattcctacgctgaatatttccagcactggggccagggtactctggtcaccgtctccagt

SEQ ID NO: 73 - DNA, 107 bp, synthetic construct
  misc_feature 1..107: note = Synthetic polynucleotide
  source 1..107: mol_type = other DNA
gcagtttattactgcgctargnnknnkggttattgttccnnknnktcttgctatactnnknnktattcctacgctgaatatttccagcactggggccagggtactct

SEQ ID NO: 74 - DNA, 18 bp, synthetic construct
  misc_feature 1..18: note = Synthetic oligonucleotide
  source 1..18: mol_type = other DNA
gcagtttattactgcgct

SEQ ID NO: 75 - DNA, 37 bp, synthetic construct
  misc_feature 1..37: note = Synthetic oligonucleotide
  source 1..37: mol_type = other DNA
agagtaccctggccccagacgtccataccgtaatagt

SEQ ID NO: 76 - DNA, 37 bp, synthetic construct
  misc_feature 1..37: note = Synthetic oligonucleotide
  source 1..37: mol_type = other DNA
actattacggtatggacgtctggggccagggtactct

SEQ ID NO: 77 - DNA, 90 bp, synthetic construct
  misc_feature 1..90: note = Synthetic oligonucleotide
  source 1..90: mol_type = other DNA
ttcactatctctagagacaactctaagaatactctctacttgcagatgaacagcttaagggctgaggatactgcagtttattactgcgct

SEQ ID NO: 78 - DNA, 33 bp, synthetic construct
  misc_feature 1..33: note = Synthetic oligonucleotide
  source 1..33: mol_type = other DNA
actggagacggtgaccagagtaccctggcccca

SEQ ID NO: 79 - DNA, 33 bp, synthetic construct
  misc_feature 1..33: note = Synthetic oligonucleotide
  source 1..33: mol_type = other DNA
tggggccagggtactctggtcaccgtctccagt

SEQ ID NO: 80 - protein, 39 aa, synthetic construct
  REGION 1..39: note = Synthetic polypeptide
YYCAXXXYYYGXGSXYNXXSYYAEYFQHWGQGTLVTVSS

SEQ ID NO: 81 - protein, 10 aa, Homo sapiens
YYYGSGSYYN

SEQ ID NO: 82 - protein, 62 aa, synthetic construct
  REGION 1..62: note = Synthetic polypeptide
  VARIANT 28: note = R or K
SRDNSKNTLYLQMNSLRAEDTAVYYCAXXXYYYGXGSXYNXXSYYAEYFQHWGQGTLVTVSS

SEQ ID NO: 83 - DNA, 195 bp, synthetic construct
  misc_feature 1..195: note = Synthetic polynucleotide
  source 1..195: mol_type = other DNA
  CDS 10..195
ttcactatctctagagacaactctaagaatactctctacttgcagatgaacagcttaagggctgaggatactgcagtttattactgcgctargnnknnktactactatggtnnkggctctnnktacaatnnknnktcttattacgctgagtactttcaacattggggccagggtactctggtcaccgtctccagt

SEQ ID NO: 84 - DNA, 107 bp, synthetic construct
  misc_feature 1..107: note = Synthetic polynucleotide
  source 1..107: mol_type = other DNA
gcagtttattactgcgctargnnknnktactactatggtnnkggctctnnktacaatnnknnktcttattacgctgagtactttcaacattggggccagggtactct

SEQ ID NO: 85 - protein, 36 aa, synthetic construct
  REGION 1..36: note = Synthetic polypeptide
  VARIANT 7: note = R or K
  VARIANT 9: note = Y or S
  VARIANT 11..12: note = Y or S
  VARIANT 14: note = Y or S
  VARIANT 16: note = Y or S
  VARIANT 20: note = Y or S
  VARIANT 22: note = Y or S
  VARIANT 24: note = Y or S
  VARIANT 27: note = Y or S
AVYYCAXXXYXXGXGXXYNXXXYXAXXFQHWGQGTL

SEQ ID NO: 86 - DNA, 107 bp, synthetic construct
  misc_feature 1..107: note = Synthetic polynucleotide
  source 1..107: mol_type = other DNA
gcagtttattactgcgctargnnktmctactmctmtggttmcggctmtnnktacaattmtnnktmctattmcgctnnktmctttcaacattggggccagggtactct

SEQ ID NO: 87 - protein, 51 aa, synthetic construct
  REGION 1..51: note = Synthetic polypeptide
  VARIANT 5: note = K or R
  VARIANT 15: note = Y or S
  VARIANT 19: note = Y or S
  VARIANT 22: note = Y or S
  VARIANT 30: note = Y or S
  VARIANT 32..34: note = Y or S
  VARIANT 36..37: note = Y or S
YYCAXYYSXSYYXYXYDSXGYXYXYYSXYXYXXXAXXFQHWGQGTLVTVSS

SEQ ID NO: 88 - protein, 10 aa, Homo sapiens
YYYDSSGYYY

SEQ ID NO: 89 - protein, 74 aa, synthetic construct
  REGION 1..74: note = Synthetic polypeptide
  VARIANT 28: note = R or K
  VARIANT 38: note = Y or S
  VARIANT 42: note = Y or S
  VARIANT 45: note = Y or S
  VARIANT 53: note = Y or S
  VARIANT 55..57: note = Y or S
  VARIANT 59..60: note = Y or S
SRDNSKNTLYLQMNSLRAEDTAVYYCAXYYSXSYYXYXYDSXGYXYXYYSXYXYXXXAXXFQHWGQGTLVTVSS

SEQ ID NO: 90 - DNA, 231 bp, synthetic construct
  misc_feature 1..231: note = Synthetic polynucleotide
  source 1..231: mol_type = other DNA
  CDS 10..231
ttcactatctctagagacaactctaagaatactctctacttgcagatgaacagcttaagggctgaggatactgcagtttattactgcgctargtactattccnnktcttactatnnktattmttacgatagttmtggttactmctatnnktactatagcnnktattmctactmctmttmcgcttmttmcttccaacactggggccagggtactctggtcaccgtctccagt

SEQ ID NO: 91 - DNA, 143 bp, synthetic construct
  misc_feature 1..143: note = Synthetic polynucleotide
  source 1..143: mol_type = other DNA
gcagtttattactgcgctargtactattccnnktcttactatnnktattmttacgatagttmtggttactmctatnnktactatagcnnktattmctactmctmttmcgcttmttmcttccaacactggggccagggtactct

SEQ ID NO: 92 - protein, 39 aa, synthetic construct
  REGION 1..39: note = Synthetic polypeptide
  VARIANT 5: note = K or R
  VARIANT 6..7: note = Y or S
  VARIANT 9: note = Y or S
  VARIANT 11..12: note = Y or S
  VARIANT 14: note = Y or S
  VARIANT 16: note = Y or S
  VARIANT 21: note = Y or S
  VARIANT 24: note = Y or S
YYCAXXXGXCXXXXCXTXXYXYXXYFQHWGQGTLVTVSS

SEQ ID NO: 93 - protein, 62 aa, synthetic construct
  REGION 1..62: note = Synthetic polypeptide
  VARIANT 28: note = R or K
  VARIANT 29..30: note = Y or S
  VARIANT 32: note = Y or S
  VARIANT 34..35: note = Y or S
  VARIANT 37: note = Y or S
  VARIANT 39: note = Y or S
  VARIANT 44: note = Y or S
  VARIANT 47: note = Y or S
SRDNSKNTLYLQMNSLRAEDTAVYYCAXXXGXCXXXXCXTXXYXYXXYFQHWGQGTLVTVSS

SEQ ID NO: 94 - DNA, 195 bp, synthetic construct
  misc_feature 1..195: note = Synthetic polynucleotide
  source 1..195: mol_type = other DNA
  CDS 10..195
ttcactatctctagagacaactctaagaatactctctacttgcagatgaacagcttaagggctgaggatactgcagtttattactgcgctargtmctmtggttmttgctmctmtnnktmttgttmcaccnnknnktattmttacnnktmttatttccagcactggggccagggtactctggtcaccgtctccagt

SEQ ID NO: 95 - DNA, 107 bp, synthetic construct
  misc_feature 1..107: note = Synthetic polynucleotide
  source 1..107: mol_type = other DNA
gcagtttattactgcgctargtmctmtggttmttgctmctmtnnktmttgttmcaccnnknnktattmttacnnktmttatttccagcactggggccagggtactct

SEQ ID NO: 96 - protein, 51 aa, synthetic construct
  REGION 1..51: note = Synthetic polypeptide
  VARIANT 5: note = K or R
  VARIANT 6: note = Y or S
  VARIANT 9: note = Y or S
  VARIANT 12: note = Y or S
  VARIANT 14..15: note = Y or S
  VARIANT 19: note = Y or S
  VARIANT 22: note = Y or S
  VARIANT 24: note = Y or S
  VARIANT 26: note = Y or S
  VARIANT 29: note = Y or S
  VARIANT 31: note = Y or S
  VARIANT 33: note = Y or S
  VARIANT 37: note = Y or S
YYCAXXYXXYGXCXXXSCXTYXSXXXYSXYXSXYAEXFQHWGQGTLVTVSS

SEQ ID NO: 97 - protein, 74 aa, synthetic construct
  REGION 1..74: note = Synthetic polypeptide
  VARIANT 28: note = R or K
  VARIANT 29: note = Y or S
  VARIANT 32: note = Y or S
  VARIANT 35: note = Y or S
  VARIANT 37..38: note = Y or S
  VARIANT 42: note = Y or S
  VARIANT 45: note = Y or S
  VARIANT 47: note = Y or S
  VARIANT 49: note = Y or S
  VARIANT 54: note = Y or S
  VARIANT 56: note = Y or S
SRDNSKNTLYLQMNSLRAEDTAVYYCAXXYXXYGXCXXXSCXTYXSXXXYSXYXSXYAEYFQHWGQGTLVTVSS

SEQ ID NO: 98 - DNA, 231 bp, synthetic construct
  misc_feature 1..231: note = Synthetic polynucleotide
  source 1..231: mol_type = other DNA
  CDS 10..231
ttcactatctctagagacaactctaagaatactctctacttgcagatgaacagcttaagggctgaggatactgcagtttattactgcgctargtmttacnnktmctacggctmttgctmttmcnnktcttgttmcacctattmttcctmtnnktmctattctnnktactmcagttmttatgctgagtatttccagcactggggccagggtactctggtcaccgtctccagt

SEQ ID NO: 99 - DNA, 143 bp, synthetic construct
  misc_feature 1..143: note = Synthetic polynucleotide
  source 1..143: mol_type = other DNA
gcagtttattactgcgctargtmttacnnktmctacggctattgctmttmcnnktmttgttmcacctattmttcctmtnnktmctattctnnktactmcagttmttatgctgagtatttccagcactggggccagggtactct

SEQ ID NO: 100 - protein, 51 aa, synthetic construct
  REGION 1..51: note = Synthetic polypeptide
  VARIANT 5: note = K or R
  VARIANT 6: note = Y or S
  VARIANT 8..9: note = Y or S
  VARIANT 12: note = Y or S
  VARIANT 14..15: note = Y or S
  VARIANT 17: note = Y or S
  VARIANT 19: note = Y or S
  VARIANT 22: note = Y or S
  VARIANT 24: note = Y or S
  VARIANT 26: note = Y or S
  VARIANT 29: note = Y or S
  VARIANT 31: note = Y or S
  VARIANT 33: note = Y or S
  VARIANT 36: note = E, K or W
  VARIANT 37: note = Y or S
  VARIANT 38: note = F, S or Y
  VARIANT 40: note = Y, H, N or D
YYCAXXYXXYGXCXXXXCXTYXSXXXYSXYXSXYAXXXQXWGQGTLVTVSS

SEQ ID NO: 101 - protein, 74 aa, synthetic construct
  REGION 1..74: note = Synthetic polypeptide
  VARIANT 28: note = R or K
  VARIANT 29: note = Y or S
  VARIANT 31..32: note = Y or S
  VARIANT 35: note = Y or S
  VARIANT 37..38: note = Y or S
  VARIANT 40: note = Y or S
  VARIANT 42: note = Y or S
  VARIANT 45: note = Y or S
  VARIANT 47: note = Y or S
  VARIANT 49: note = Y or S
  VARIANT 52: note = Y or S
  VARIANT 54: note = Y or S
  VARIANT 56: note = Y or S
  VARIANT 59: note = K, E or Q
  VARIANT 60: note = Y or S
  VARIANT 61: note = Y, S or F
  VARIANT 63: note = N, D, H or Y
SRDNSKNTLYLQMNSLRAEDTAVYYCAXXYXXYGXCXXXXCXTYXSXXXYSXYXSXYAXXXQXWGQGTLVTVSS

SEQ ID NO: 102 - DNA, 231 bp, synthetic construct
  misc_feature 1..231: note = Synthetic polynucleotide
  source 1..231: mol_type = other DNA
  CDS 10..231
ttcactatctctagagacaactctaagaatactctctacttgcagatgaacagcttaagggctgaggatactgcagtttattactgcgctargtmttactmctmctacggctmttgctmttmcnnktmttgttmcacctattmttcctmtnnktmctattcttmttactmcagttmttatgctvagtmtthccagnactggggccagggtactctggtcaccgtctccagt

SEQ ID NO: 103 - protein, 50 aa, synthetic construct
  REGION 1..50: note = Synthetic polypeptide
  VARIANT 5: note = K or R
  VARIANT 6: note = Y or S
  VARIANT 8: note = Y or S
  VARIANT 10: note = Y or S
  VARIANT 11: note = Y, H, N or D
  VARIANT 12: note = Y or S
  VARIANT 16..17: note = Y or S
  VARIANT 19: note = Y or S
  VARIANT 21: note = Y or S
  VARIANT 23: note = Y or S
  VARIANT 25: note = Y or S
  VARIANT 27..29: note = Y or S
  VARIANT 31: note = Y or S
  VARIANT 33: note = Y or S
  VARIANT 35: note = E, K or Q
  VARIANT 36: note = Y or S
YYCAXXXXYXXXVWGXXRXTXSXXXYXXXYXSXAXXFQHWGQGTLVTVSS

SEQ ID NO: 104 - protein, 12 aa, Homo sapiens
YYDYVWGSYRYT

SEQ ID NO: 105 - protein, 73 aa, synthetic construct
  REGION 1..73: note = Synthetic polypeptide
  VARIANT 28: note = R or K
  VARIANT 29: note = Y or S
  VARIANT 31: note = Y or S
  VARIANT 33: note = Y or S
  VARIANT 34: note = N, D, H or Y
  VARIANT 35: note = Y or S
  VARIANT 39..40: note = Y or S
  VARIANT 42: note = Y or S
  VARIANT 44: note = Y or S
  VARIANT 46: note = Y or S
  VARIANT 48: note = Y or S
  VARIANT 50..52: note = Y or S
  VARIANT 54: note = Y or S
  VARIANT 56: note = Y or S
  VARIANT 58: note = K, E or Q
  VARIANT 59: note = Y or S
SRDNSKNTLYLQMNSLRAEDTAVYYCAXXXXYXXXVWGXXRXTXSXXXYXXXYXSXAXXFQHWGQGTLVTVSS

SEQ ID NO: 106 - DNA, 228 bp, synthetic construct
  misc_feature 1..228: note = Synthetic polynucleotide
  source 1..228: mol_type = other DNA
  CDS 10..228
ttcactatctctagagacaactctaagaatactctctacttgcagatgaacagcttaagggctgaggatactgcagtttattactgcgctargtmtnnktmctactmtnattmtgtttggggttmttmccgttmtacttmtagttmcnnktmttactmctmttmctattmcagttmtgctvagtmcttccagcattggggccagggtactctggtcaccgtctccagt

SEQ ID NO: 107 - DNA, 140 bp, synthetic construct
  misc_feature 1..140: note = Synthetic polynucleotide
  source 1..140: mol_type = other DNA
gcagtttattactgcgctargtmtnnktmctactmtnattmtgtttggggttmttmccgttmtacttmtagttmcnnktmttactmctmttmctattmcagttmtgctvagtmcttccagcattggggccagggtactct

SEQ ID NO: 108 - protein, 50 aa, synthetic construct
  REGION 1..50: note = Synthetic polypeptide
  VARIANT 5: note = K or R
  VARIANT 6: note = Y or S
  VARIANT 8..10: note = Y or S
  VARIANT 12: note = Y or S
  VARIANT 14..16: note = Y or S
  VARIANT 18: note = Y or S
  VARIANT 19: note = Y, H, N or D
  VARIANT 20: note = Y or S
  VARIANT 24..25: note = Y or S
  VARIANT 27: note = Y or S
  VARIANT 30: note = Y or S
  VARIANT 32: note = Y or S
  VARIANT 35: note = E, K or Q
  VARIANT 36: note = Y or S
YYCAXXXXXXXXYXXXYXXXVWGXXRXTYXSXYAXXFQHWGQGTLVTVSS

SEQ ID NO: 109 - protein, 73 aa, synthetic construct
  REGION 1..73: note = Synthetic polypeptide
  VARIANT 28: note = R or K
  VARIANT 29: note = Y or S
  VARIANT 31..33: note = Y or S
  VARIANT 35: note = Y or S
  VARIANT 37..39: note = Y or S
  VARIANT 41: note = Y or S
  VARIANT 42: note = N, D, H or Y
  VARIANT 43: note = Y or S
  VARIANT 47..48: note = Y or S
  VARIANT 50: note = Y or S
  VARIANT 53: note = Y or S
  VARIANT 55: note = Y or S
  VARIANT 58: note = K, E or Q
  VARIANT 59: note = Y or S
SRDNSKNTLYLQMNSLRAEDTAVYYCAXXXXXXXXYXXXYXXXVWGXXRXTYXSXYAXXFQHWGQGTLVTVSS

SEQ ID NO: 110 - DNA, 228 bp, synthetic construct
  misc_feature 1..228: note = Synthetic polynucleotide
  source 1..228: mol_type = other DNA
  CDS 10..228
ttcactatctctagagacaactctaagaatactctctacttgcagatgaacagcttaagggctgaggatactgcagtttattactgcgctargtmtnnktmctmttmcnnktmttactmctmttmctactmtnattmtgtttggggttmttmccgttmtacttattmcagttmttacgctvagtmcttccagcattggggccagggtactctggtcaccgtctccagt

SEQ ID NO: 111 - DNA, 140 bp, synthetic construct
  misc_feature 1..140: note = Synthetic polynucleotide
  source 1..140: mol_type = other DNA
gcagtttattactgcgctargtmtnnktmctmttmcnnktmttactmctmttmctactmtnattmtgtttggggttmttmccgttmtacttattmcagttmttacgctvagtmcttccagcattggggccagggtactct

SEQ ID NO: 112 - protein, 40 aa, synthetic construct
  REGION 1..40: note = Synthetic polypeptide
  VARIANT 5: note = K or R
  VARIANT 7..8: note = Y or S
  VARIANT 10..11: note = Y or S
  VARIANT 13: note = Y or S
  VARIANT 15: note = Y or S
  VARIANT 19: note = Y or S
  VARIANT 21: note = Y or S
  VARIANT 23: note = Y or S
  VARIANT 26: note = Y or S
YYCAXXXXYXXGXGXXYNXXXYXAXXFQHWGQGTLVTVSS

SEQ ID NO: 113 - DNA, 110 bp, synthetic construct
  misc_feature 1..110: note = Synthetic polynucleotide
  source 1..110: mol_type = other DNA
gcagtttattactgcgctargnnktmctmctactmctmtggttmcggctmtnnktacaattmtnnktmctattmcgctnnktmctttcaacattggggccagggtactct

SEQ ID NO: 114 - protein, 39 aa, synthetic construct
  REGION 1..39: note = Synthetic polypeptide
  VARIANT 5: note = R or K
  VARIANT 6..7: note = Y or S
  VARIANT 9: note = Y or S
  VARIANT 11: note = Y or S
  VARIANT 13: note = I, T, K or R
  VARIANT 14: note = Y, H, N or D
  VARIANT 18: note = Y or S
  VARIANT 19: note = I, T, K or R
  VARIANT 20: note = Y or S
  VARIANT 22..23: note = Y or S
  VARIANT 24: note = K, S or W
  VARIANT 25: note = Y or S
  VARIANT 26: note = F, Y or S
  VARIANT 27: note = Y, H, N or D
YYCAXXXXXGXCXXGVCXXXXXXXXXXLWGRGTLVTVSS

SEQ ID NO: 115 - protein, 10 aa, Homo sapiens
GYCTNGVCYT

SEQ ID NO: 116 - DNA, 107 bp, synthetic construct
  misc_feature 1..107: note = Synthetic polynucleotide
  source 1..107: mol_type = other DNA
gcagtttattactgcgctargtmctmtnnktmtggttmctgtananatggtgtctgctmtanatmcnnktmttmttbgtmtthtnatctgtggggccagggtactct

SEQ ID NO: 117 - DNA, 107 bp, synthetic construct
  misc_feature 1..107: note = Synthetic polynucleotide
  source 1..107: mol_type = other DNA
gcagtttattactgcgctargtmctmtnnktmcggttmctgcananatggcgtctgctmtanatmcnnktmttmttbgtmtthtnatctgtggggccagggtactct

SEQ ID NO: 118 - DNA, 58 bp, synthetic construct
  misc_feature 1..58: note = Synthetic oligonucleotide
  source 1..58: mol_type = other DNA
gcagtttattactgcgctargtmctmtnnktmcggttmctgcananatggcgtctgct

SEQ ID NO: 119 - DNA, 75 bp, synthetic construct
  misc_feature 1..75: note = Synthetic oligonucleotide
  source 1..75: mol_type = other DNA
agagtaccctggccccacagatnadaakacvaakaakamnngkatntakagcagacgccatntntgcagkaaccg

SEQ ID NO: 120 - DNA, 75 bp, synthetic construct
  misc_feature 1..75: note = Synthetic oligonucleotide
  source 1..75: mol_type = other DNA
cggttmctgcananatggcgtctgctmtanatmcnnktmttmttbgtmtthtnatctgtggggccagggtactct

SEQ ID NO: 121 - protein, 49 aa, synthetic construct
  REGION 1..49: note = Synthetic polypeptide
  VARIANT 5: note = R or K
  VARIANT 6..11: note = Y or S
  VARIANT 13: note = Y or S
  VARIANT 15: note = I, T, K or R
  VARIANT 16: note = Y, H, N or D
  VARIANT 20: note = Y or S
  VARIANT 21: note = I, T, K or R
  VARIANT 22..25: note = Y or S
  VARIANT 27..28: note = Y or S
  VARIANT 30: note = Y or S
  VARIANT 32..33: note = Y or S
  VARIANT 34: note = K, S or W
  VARIANT 35: note = Y or S
  VARIANT 36: note = F, Y or S
  VARIANT 37: note = Y, H, N or D
YYCAXXXXXXXGXCXXGVCXXXXXXYXXYXYXXXXXXLWGRGTLVTVSS

SEQ ID NO: 122 - DNA, 137 bp, synthetic construct
  misc_feature 1..137: note = Synthetic polynucleotide
  source 1..137: mol_type = other DNA
gcagtttattactgcgctargtmttmctmctmttmctmcggttmttgtananatggcgtgtgctmtanatmctmctmctmttattmttmctattmttactmttmctbgtmcthtnatctgtggggccagggtactct

SEQ ID NO: 123 - protein, 50 aa, synthetic construct
  REGION 1..50: note = Synthetic polypeptide
  VARIANT 5: note = R or K
  VARIANT 6..15: note = Y or S
  VARIANT 26..34: note = Y or S
  VARIANT 36: note = Y or S
  VARIANT 38: note = Y, H, N or D
YYCAXXXXXXXXXXXGYCTNGVCYTXXXXXXXXXWXFXLWGRGTLVTVSS

SEQ ID NO: 124 - DNA, 81 bp, synthetic construct
  misc_feature 1..81: note = Synthetic oligonucleotide
  source 1..81: mol_type = other DNA
gcagtttattactgcgctargtmttmctmctmttmttmctmctmttmctmcggttattgtactaacggcgtttgctatact

SEQ ID NO: 125 - DNA, 86 bp, synthetic construct
  misc_feature 1..86: note = Synthetic oligonucleotide
  source 1..86: mol_type = other DNA
agagtaccctggccccacaggtngaaakaccaakaakaakagkagkagkagkaakaakaagtatagcaaacgccgttagtacaata

SEQ ID NO: 126 - DNA, 86 bp, synthetic construct
  misc_feature 1..86: note = Synthetic oligonucleotide
  source 1..86: mol_type = other DNA
tattgtactaacggcgtttgctatacttmttmttmctmctmctmctmttmttmttggtmtttcnacctgtggggccagggtactct

SEQ ID NO: 127 - protein, 50 aa, synthetic construct
  REGION 1..50: note = Synthetic polypeptide
  VARIANT 5: note = K or R
  VARIANT 6..16: note = Y or S
  VARIANT 25: note = Y or S
  VARIANT 27..33: note = Y or S
  VARIANT 36: note = Y or S
YYCAXXXXXXXXXXXXDYVWGSYRXTXXXXXXXAEXFQHWGQGTLVTVSS

SEQ ID NO: 128 - DNA, 78 bp, synthetic construct
  misc_feature 1..78: note = Synthetic oligonucleotide
  source 1..78: mol_type = other DNA
gcagtttattactgcgctargtmttmctmctmttmttmctmctmttmctmctmcgattatgtctggggtacttatcgt

SEQ ID NO: 129 - DNA, 86 bp, synthetic construct
  misc_feature 1..86: note = Synthetic oligonucleotide
  source 1..86: mol_type = other DNA
agagtaccctggccccaatgctggaaakactcagcgkagkagkagkagkagkaakaagtgkaacgataagtaccccagacataatc

SEQ ID NO: 130 - DNA, 86 bp, synthetic construct
  misc_feature 1..86: note = Synthetic oligonucleotide
  source 1..86: mol_type = other DNA
gattatgtctggggtacttatcgttmcacttmttmctmctmctmctmctmcgctgagtmtttccagcattggggccagggtactct

SEQ ID NO: 131 - protein, 51 aa, synthetic construct
  REGION 1..51: note = Synthetic polypeptide
  VARIANT 6..16: note = Y or S
  VARIANT 18: note = Y or S
  VARIANT 22..23: note = Y or S
  VARIANT 25: note = Y or S
  VARIANT 27..34: note = Y or S
  VARIANT 37: note = Y or S
YYCAKXXXXXXXXXXXDXVWGXXRXTXXXXXXXXAEXFQHWGQGTLVTVSS

SEQ ID NO: 132 - DNA, 85 bp, synthetic construct
  misc_feature 1..85: note = Synthetic oligonucleotide
  source 1..85: mol_type = other DNA
gcagtttattactgcgctargtmttmctmctmttmttmctmctmttmctmctmcgactmtgtctggggttmctmccgttmcacct

SEQ ID NO: 133 - DNA, 93 bp, synthetic construct
  misc_feature 1..93: note = Synthetic oligonucleotide
  source 1..93: mol_type = other DNA
agagtaccctggccccaatgctggaaakactcagcgkagkagkagkagkagkagkaakaggtgkaacggkagkaaccccagacakagtcgkag

SEQ ID NO: 134 - DNA, 93 bp, synthetic construct
  misc_feature 1..93: note = Synthetic oligonucleotide
  source 1..93: mol_type = other DNA
ctmcgactmtgtctggggttmctmccgttmcacctmttmctmctmctmctmctmctmcgctgagtmtttccagcattggggccagggtactct

SEQ ID NO: 135 - protein, 51 aa, synthetic construct
  REGION 1..51: note = Synthetic polypeptide
  VARIANT 5: note = K or R
  VARIANT 6..15: note = Y or S
  VARIANT 17: note = Y or S
  VARIANT 19: note = Y or S
  VARIANT 22: note = Y or S
  VARIANT 24: note = Y or S
  VARIANT 26..34: note = Y or S
  VARIANT 37: note = Y or S
YYCAXXXXXXXXXXXGXCXGGXCXSXXXXXXXXXAEXFQHWGQGTLVTVSS

SEQ ID NO: 136 - protein, 10 aa, Homo sapiens
GYCSGGSCYS

SEQ ID NO: 137 - DNA, 82 bp, synthetic construct
  misc_feature 1..82: note = Synthetic oligonucleotide
  source 1..82: mol_type = other DNA
gcagtttattactgcgctargtmttmttmttmttmttmttmttmttmctmcggctmctgttmcggtggctmctgctmctcct

SEQ ID NO: 138 - DNA, 96 bp, synthetic construct
  misc_feature 1..96: note = Synthetic oligonucleotide
  source 1..96: mol_type = other DNA
agagtaccctggccccaatgttggaaakattcagcgkagkagkagkagkagkagkagkagkagkaggagcagkagccaccgkaacagkagccgkag

SEQ ID NO: 139 - DNA, 79 bp, synthetic construct
  misc_feature 1..79: note = Synthetic oligonucleotide
  source 1..79: mol_type = other DNA
gcagtttattactgcgctargtmttmttmttmttmttmttmttmctmcggctmctgttmcggtggctmctgctmctcct

SEQ ID NO: 140 - DNA, 93 bp, synthetic construct
  misc_feature 1..93: note = Synthetic oligonucleotide
  source 1..93: mol_type = other DNA
agagtaccctggccccaatgttggaaakattcagcgkagkagkagkagkagkagkagkagkaggagcagkagccaccgkaacagkagccgkag

SEQ ID NO: 141 - protein, 50 aa, synthetic construct
  REGION 1..50: note = Synthetic polypeptide
  VARIANT 5: note = K or R
  VARIANT 6..12: note = Y or S
  VARIANT 14: note = Y or S
  VARIANT 18..19: note = Y or S
  VARIANT 21: note = Y or S
  VARIANT 23..33: note = Y or S
  VARIANT 36: note = Y or S
YYCAXXXXXXXXDXVWGXXRXTXXXXXXXXXXXAEXFQHWGQGTLVTVSS

SEQ ID NO: 142 - DNA, 73 bp, synthetic construct
  misc_feature 1..73: note = Synthetic oligonucleotide
  source 1..73: mol_type = other DNA
gcagtttattactgcgctargtmttmttmttmttmttmttmcgactmcgtctggggttmttmccgttmtacct

SEQ ID NO: 143 - DNA, 105 bp, synthetic construct
  misc_feature 1..105: note = Synthetic polynucleotide
  source 1..105: mol_type = other DNA
agagtaccctggccccagtgctggaagkactcagcgkagkagkagkagkagkagkagkagkagkagkagkagkaggtakaacggkaakaaccccagacgkagtcg

SEQ ID NO: 144 - protein, 50 aa, synthetic construct
  REGION 1..50: note = Synthetic polypeptide
  VARIANT 5: note = K or R
  VARIANT 6..15: note = Y or S
  VARIANT 17: note = Y or S
  VARIANT 21..22: note = Y or S
  VARIANT 24: note = Y or S
  VARIANT 26..33: note = Y or S
  VARIANT 36: note = Y or S
YYCAXXXXXXXXXXXDXVWGXXRXTXXXXXXXXAEXFQHWGQGTLVTVSS

SEQ ID NO: 145 - DNA, 82 bp, synthetic construct
  misc_feature 1..82: note = Synthetic oligonucleotide
  source 1..82: mol_type = other DNA
gcagtttattactgcgctargtmttmttmttmttmttmttmctmctmctmcgactmcgtctggggttmctmccgttmcacct

SEQ ID NO: 146 - DNA, 96 bp, synthetic construct
  misc_feature 1..96: note = Synthetic oligonucleotide
  source 1..96: mol_type = other DNA
agagtaccctggccccagtgctggaagkactcagcgkagkagkagkagkagkagkagkagkagkaggtgkaacggkagkaaccccagacgkagtcg

SEQ ID NO: 147 - protein, 51 aa, synthetic construct
  REGION 1..51: note = Synthetic polypeptide
  VARIANT 5: note = K or R
  VARIANT 6..16: note = Y or S
  VARIANT 21..34: note = Y or S
  VARIANT 37: note = Y or S
YYCAXXXXXXXXXXXXDSSGXXXXXXXXXXXXXXAEXFQHWGQGTLVTVSS

SEQ ID NO: 148 - protein, 74 aa, synthetic construct
  REGION 1..74: note = Synthetic polypeptide
  VARIANT 28: note = K or R
  VARIANT 29..39: note = Y or S
  VARIANT 44..57: note = Y or S
  VARIANT 60: note = Y or S
SRDNSKNTLYLQMNSLRAEDTAVYYCAXXXXXXXXXXXXDSSGXXXXXXXXXXXXXXAEXFQHWGQGTLVTVSS

SEQ ID NO: 149 - DNA, 231 bp, synthetic construct
  misc_feature 1..231: note = Synthetic polynucleotide
  source 1..231: mol_type = other DNA
  CDS 10..231
ttcactatctctagagacaactctaagaatactctctacttgcagatgaacagcttaagggctgaggatactgcagtttattactgcgctargtmctmttmctmctmttmctmttmctmctmctmcgacagctccggctmctmctmttmctmttmctmctmttmctmttmctmctmctmcgctgaatmcttccaacactggggccagggtactctggtcaccgtctccagt

SEQ ID NO: 150 - DNA, 73 bp, synthetic construct
  misc_feature 1..73: note = Synthetic oligonucleotide
  source 1..73: mol_type = other DNA
gcagtttattactgcgctargtmctmttmctmctmttmctmttmctmctmctmcgacagctccggctmctmct

SEQ ID NO: 151 - DNA, 97 bp, synthetic construct
  misc_feature 1..97: note = Synthetic oligonucleotide
  source 1..97: mol_type = other DNA
cagagtaccctggccccagtgttggaagkattcagcgkagkagkagkaakagkaakagkagkaakagkaakagkagkagccggagctgtcgkagkag

SEQ ID NO: 152 - protein, 51 aa, synthetic construct; identical to SEQ ID NO: 147
  REGION 1..51: note = Synthetic polypeptide
  VARIANT 5: note = K or R
  VARIANT 6..16: note = Y or S
  MOD_RES 12: note = May or may not be present
  VARIANT 21..34: note = Y or S
  MOD_RES 31: note = May or may not be present
  VARIANT 37: note = Y or S

SEQ ID NO: 153 - DNA, 73 bp, synthetic construct; identical to SEQ ID NO: 150
  misc_feature 1..73: note = Synthetic oligonucleotide
  source 1..73: mol_type = other DNA

SEQ ID NO: 154 - DNA, 70 bp, synthetic construct
  misc_feature 1..70: note = Synthetic oligonucleotide
  source 1..70: mol_type = other DNA
gcagtttattactgcgctargtmctmctmctmttmctmttmctmctmctmcgacagctccggctmctmct

SEQ ID NO: 155 - DNA, 94 bp, synthetic construct
  misc_feature 1..94: note = Synthetic oligonucleotide
  source 1..94: mol_type = other DNA
cagagtaccctggccccagtgttggaagkattcagcgkagkagkagkaakagkaakagkagkaakaakagkagkagccggagctgtcgkagkag

SEQ ID NO: 156 - protein, 5 aa, Homo sapiens
GTTGT

SEQ ID NO: 157 - protein, 5 aa, Homo sapiens
VQLER

SEQ ID NO: 158 - protein, 5 aa, Homo sapiens
YNWND

SEQ ID NO: 159 - protein, 5 aa, Homo sapiens
GITGT

SEQ ID NO: 160 - skipped

SEQ ID NO: 161 - protein, 5 aa, Homo sapiens
YNWNY

SEQ ID NO: 162 - skipped

SEQ ID NO: 163 - protein, 5 aa, Homo sapiens
YNWND

SEQ ID NO: 164 - protein, 6 aa, Homo sapiens
GIVGAT

SEQ ID NO: 165 - protein, 4 aa, Homo sapiens
WELL

SEQ ID NO: 166 - protein, 6 aa, Homo sapiens
YSGSYY

SEQ ID NO: 167 - protein, 5 aa, Homo sapiens
YQLLY

SEQ ID NO: 168 - protein, 9 aa, Homo sapiens
DIVVVPAAI

SEQ ID NO: 169 - protein, 4 aa, Homo sapiens
RILY

SEQ ID NO: 170 - protein, 9 aa, Homo sapiens
DIVLMVYAI

SEQ ID NO: 171 - skipped

SEQ ID NO: 172 - protein, 9 aa, Homo sapiens
DIVVVVAAT

SEQ ID NO: 173 - protein, 5 aa, Homo sapiens
SILWW

SEQ ID NO: 174 - protein, 9 aa, Homo sapiens
AYCGGDCYS

SEQ ID NO: 175 - protein, 8 aa, Homo sapiens
HIVVVTAI

SEQ ID NO: 176 - protein, 10 aa, Homo sapiens
VLRFLEWLLY

SEQ ID NO: 177 - protein, 10 aa, Homo sapiens
YYDFWSGYYT

SEQ ID NO: 178 - protein, 9 aa, Homo sapiens
ITIFGVVII

SEQ ID NO: 179 - protein, 9 aa, Homo sapiens
VLRYFDWLL

SEQ ID NO: 180 - protein, 10 aa, Homo sapiens
YYDILTGYYN

SEQ ID NO: 181 - protein, 4 aa, Homo sapiens
ITIF

SEQ ID NO: 182 - protein, 9 aa, Homo sapiens
VLLWFGELL

SEQ ID NO: 183 - protein, 9 aa, Homo sapiens
ITMVRGVII

SEQ ID NO: 184 - protein, 9 aa, Homo sapiens
LRLGELSLY

SEQ ID NO: 185 - protein, 11 aa, Homo sapiens
IMITFGGVIVI

SEQ ID NO: 186 - protein, 4 aa, Homo sapiens
WLLL

SEQ ID NO: 187 - protein, 10 aa, Homo sapiens
YYYDSSGYYY

SEQ ID NO: 188 - protein, 9 aa, Homo sapiens
ITMIVVVIT

SEQ ID NO: 189 - skipped

SEQ ID NO: 190 - protein, 4 aa, Homo sapiens
TTVT

SEQ ID NO: 191 - skipped

SEQ ID NO: 192 - protein, 5 aa, Homo sapiens
DYSNY

SEQ ID NO: 193 - protein, 4 aa, Homo sapiens
TTVT

SEQ ID NO: 194 - skipped

SEQ ID NO: 195 - protein, 5 aa, Homo sapiens
DYGDY

SEQ ID NO: 196 - protein, 4 aa, Homo sapiens
TTVT

SEQ ID NO: 197 - skipped

SEQ ID NO: 198 - protein, 6 aa, Homo sapiens
DYGGNS

SEQ ID NO: 199 - protein, 5 aa, Homo sapiens
TTVVT

SEQ ID NO: 200 - protein, 6 aa, Homo sapiens
VDTAMV

SEQ ID NO: 201 - protein, 6 aa, Homo sapiens
WIQLWL

SEQ ID NO: 202 - protein, 6 aa, Homo sapiens
GYSYGY

SEQ ID NO: 203 - protein, 7 aa, Homo sapiens
VDIVATI

SEQ ID NO: 204 - protein, 4 aa, Homo sapiens
WLRL

SEQ ID NO: 205 - protein, 7 aa, Homo sapiens
GYSGYDY

SEQ ID NO: 206 - protein, 6 aa, Homo sapiens
VDTAMV

SEQ ID NO: 207 - protein, 6 aa, Homo sapiens
WIQLWL

SEQ ID NO: 208 - protein, 6 aa, Homo sapiens
GYSYGY

SEQ ID NO: 209 - protein, 6 aa, Homo sapiens
VEMATI

SEQ ID NO: 210 - protein, 5 aa, Homo sapiens
RWLQL

SEQ ID NO: 211 - protein, 6 aa, Homo sapiens
RDGYNY

SEQ ID NO: 212 - protein, 6 aa, Homo sapiens
EYSSSS

SEQ ID NO: 213 - protein, 5 aa, Homo sapiens
SIAAR

SEQ ID NO: 214 - skipped

SEQ ID NO: 215 - protein, 7 aa, Homo sapiens
GYSSSWY

SEQ ID NO: 216 - protein, 6 aa, Homo sapiens
GIAAAG

SEQ ID NO: 217 - protein, 4 aa, Homo sapiens
QQLV

SEQ ID NO: 218 - protein, 7 aa, Homo sapiens
GYSSGWY

SEQ ID NO: 219 - protein, 6 aa, Homo sapiens
GIAVAG

SEQ ID NO: 220 - protein, 4 aa, Homo sapiens
QWLV

SEQ ID NO: 221-223 - skipped (3 entries)

SEQ ID NO: 224 - protein, 27 aa, synthetic construct
  REGION 1..27: note = Synthetic peptide
AEDTAVYYCAKDIVLMWGQGTTVTVSS

SEQ ID NO: 225 - DNA, 81 bp, synthetic construct; identical to SEQ ID NO: 55
  misc_feature 1..81: note = Synthetic oligonucleotide
  source 1..81: mol_type = other DNA

SEQ ID NO: 226 - protein, 22 aa, synthetic construct
  REGION 1..22: note = Synthetic peptide
YYCAKAEYFQHWGQGTLVTVSS

SEQ ID NO: 227 - protein, 22 aa, synthetic construct
  REGION 1..22: note = Synthetic peptide
YYCAKYDYGDYWGQGTLVTVSS

SEQ ID NO: 228 - protein, 22 aa, synthetic construct
  REGION 1..22: note = Synthetic peptide
YYCAKGYSYGYWGQGTLVTVSS

SEQ ID NO: 229 - protein, 22 aa, synthetic construct
  REGION 1..22: note = Synthetic peptide
YYCAKSYYFDYWGQGTLVTVSS

SEQ ID NO: 230 - protein, 24 aa, synthetic construct
  REGION 1..24: note = Synthetic peptide
YYCAKYYAEYFQHWGQGTLVTVSS

SEQ ID NO: 231 - protein, 24 aa, synthetic construct
  REGION 1..24: note = Synthetic peptide
YYCAKYGYSSSWYWGQGTLVTVSS

SEQ ID NO: 232 - protein, 24 aa, synthetic construct
  REGION 1..24: note = Synthetic peptide
YYCAKYGDYYFDYWGQGTLVTVSS

SEQ ID NO: 233 - protein, 26 aa, synthetic construct
  REGION 1..26: note = Synthetic peptide
YYCAKYYYDSSGYYYWGQGTLVTVSS

SEQ ID NO: 234 - protein, 26 aa, synthetic construct
  REGION 1..26: note = Synthetic peptide
YYCAKGYCSSTSCYTWGQGTLVTVSS

SEQ ID NO: 235 - protein, 26 aa, synthetic construct
  REGION 1..26: note = Synthetic peptide
YYCAKYYSSAEYFQHWGQGTLVTVSS

SEQ ID NO: 236 - protein, 26 aa, synthetic construct
  REGION 1..26: note = Synthetic peptide
YYCAKGYSYGYYFDYWGQGTLVTVSS

SEQ ID NO: 237 - protein, 28 aa, synthetic construct
  REGION 1..28: note = Synthetic peptide
YYCAKYYYDSSGYYYQHWGQGTLVTVSS

SEQ ID NO: 238 - protein, 28 aa, synthetic construct
  REGION 1..28: note = Synthetic peptide
YYCAKGYCSSTSCYTQHWGQGTLVTVSS

SEQ ID NO: 239 - protein, 28 aa, synthetic construct
  REGION 1..28: note = Synthetic peptide
YYCAKYYSSYSAEYFQHWGQGTLVTVSS

SEQ ID NO: 240 - protein, 28 aa, synthetic construct
  REGION 1..28: note = Synthetic peptide
YYCAKYYDYVWGSYRYTWGQGTLVTVSS

SEQ ID NO: 241 - protein, 28 aa, synthetic construct
  REGION 1..28: note = Synthetic peptide
YYCAKGYSYGYYWYFDLWGRGTLVTVSS

SEQ ID NO: 242 - protein, 30 aa, synthetic construct
  REGION 1..30: note = Synthetic polypeptide
YYCAKYYYDSSGYYYYFQHWGQGTLVTVSS

SEQ ID NO: 243 - protein, 30 aa, synthetic construct
  REGION 1..30: note = Synthetic polypeptide
YYCAKGYCSSTSCYTYFQHWGQGTLVTVSS

SEQ ID NO: 244 - protein, 30 aa, synthetic construct
  REGION 1..30: note = Synthetic polypeptide
YYCAKSYGYCSSTSCYTQHWGQGTLVTVSS

SEQ ID NO: 245 - protein, 30 aa, synthetic construct
  REGION 1..30: note = Synthetic polypeptide
YYCAKSYYYSSYSAEYFQHWGQGTLVTVSS

SEQ ID NO: 246 - protein, 30 aa, synthetic construct
  REGION 1..30: note = Synthetic polypeptide
YYCAKAYCGGDCYSNWFDPWGQGTLVTVSS

SEQ ID NO: 247 - protein, 32 aa, synthetic construct
  REGION 1..32: note = Synthetic polypeptide
YYCAKYYYDSSGYYYAEYFQHWGQGTLVTVSS

SEQ ID NO: 248 - protein, 32 aa, synthetic construct
  REGION 1..32: note = Synthetic polypeptide
YYCAKGYCSSTSCYTAEYFQHWGQGTLVTVSS

SEQ ID NO: 249 - protein, 32 aa, synthetic construct
  REGION 1..32: note = Synthetic polypeptide
YYCAKSYYSYSSYYSAEYFQHWGQGTLVTVSS

SEQ ID NO: 250 - protein, 32 aa, synthetic construct
  REGION 1..32: note = Synthetic polypeptide
YYCAKSYSYGYCSSTSCYTQHWGQGTLVTVSS

SEQ ID NO: 251 - protein, 36 aa, synthetic construct
  REGION 1..36: note = Synthetic polypeptide
YYCAKYSSYYYYDSSGYYYAEYFQHWGQGTLVTVSS

SEQ ID NO: 252 - protein, 36 aa, synthetic construct
  REGION 1..36: note = Synthetic polypeptide
YYCAKSYYSGYCSSTSCYTAEYFQHWGQGTLVTVSS

SEQ ID NO: 253 - protein, 36 aa, synthetic construct
  REGION 1..36: note = Synthetic polypeptide
YYCAKSGYCSSTSCYTYYSAEYFQHWGQGTLVTVSS

SEQ ID NO: 254 - protein, 36 aa, synthetic construct
  REGION 1..36: note = Synthetic polypeptide
YYCAKYYYYDYVWGSYRYTSNWFDPWGQGTLVTVSS

SEQ ID NO: 255 - protein, 36 aa, synthetic construct
  REGION 1..36: note = Synthetic polypeptide
YYCAKYYYYDYVWGSYRYTSSYFDYWGQGTLVTVSS

SEQ ID NO: 256 - DNA, 33 bp, synthetic construct
  misc_feature 1..33: note = Synthetic oligonucleotide
  source 1..33: mol_type = other DNA
actggagacggtgaccgtagtaccctggcccca

SEQ ID NO: 257 - protein, 4 aa, Homo sapiens
GTTG

SEQ ID NO: 258 - protein, 4 aa, Homo sapiens
VQLE

SEQ ID NO: 259 - protein, 4 aa, Homo sapiens
YNWN

SEQ ID NO: 260 - protein, 5 aa, Homo sapiens
GTTGT

SEQ ID NO: 261 - protein, 5 aa, Homo sapiens
VQLER

SEQ ID NO: 262 - protein, 5 aa, Homo sapiens
YNWND

SEQ ID NO: 263 - protein, 4 aa, Homo sapiens
TTGT

SEQ ID NO: 264 - protein, 4 aa, Homo sapiens
QLER

SEQ ID NO: 265 - protein, 4 aa, Homo sapiens
NWND

SEQ ID NO: 266 - protein, 4 aa, Homo sapiens
GITG

SEQ ID NO: 267 - protein, 4 aa, Homo sapiens
VYLE

SEQ ID NO: 268 - protein, 5 aa, Homo sapiens
GITGT

SEQ ID NO: 269 - protein, 5 aa, Homo sapiens
VYLEL

SEQ ID NO: 270 - protein, 5 aa, Homo sapiens
YNWNY

SEQ ID NO: 271 - protein, 4 aa, Homo sapiens
ITGT

SEQ ID NO: 272 - protein, 4 aa, Homo sapiens
YLEL

SEQ ID NO: 273 - protein, 4 aa, Homo sapiens
NWNY

SEQ ID NO: 274 - protein, 5 aa, Homo sapiens
VYLER

SEQ ID NO: 275 - protein, 4 aa, Homo sapiens
YLER

SEQ ID NO: 276 - protein, 4 aa, Homo sapiens
GIVG

SEQ ID NO: 277 - protein, 4 aa, Homo sapiens
VYWE

SEQ ID NO: 278 - protein, 4 aa, Homo sapiens
YSGS

SEQ ID NO: 279 - protein, 5 aa, Homo sapiens
GIVGA

SEQ ID NO: 280 - protein, 5 aa, Homo sapiens
VYWEL

SEQ ID NO: 281 - protein, 5 aa, Homo sapiens
YSGSY

SEQ ID NO: 282 - protein, 6 aa, Homo sapiens
GIVGAT

SEQ ID NO: 283 - protein, 6 aa, Homo sapiens
VYWELL

SEQ ID NO: 284 - protein, 6 aa, Homo sapiens
YSGSYY

SEQ ID NO: 285 - protein, 4 aa, Homo sapiens
IVGA

SEQ ID NO: 286 - protein, 4 aa, Homo sapiens
YWEL

SEQ ID NO: 287 - protein, 4 aa, Homo sapiens
SGSY

SEQ ID NO: 288 - protein, 5 aa, Homo sapiens
IVGAT

SEQ ID NO: 289 - protein, 5 aa, Homo sapiens
YWELL

SEQ ID NO: 290 - protein, 5 aa, Homo sapiens
SGSYY

SEQ ID NO: 291 - protein, 4 aa, Homo sapiens
VGAT

SEQ ID NO: 292 - protein, 4 aa, Homo sapiens
WELL

SEQ ID NO: 293 - protein, 4 aa, Homo sapiens
GSYY

SEQ ID NO: 294 - protein, 4 aa, Homo sapiens
RILY

SEQ ID NO: 295 - protein, 4 aa, Homo sapiens
GYCS

SEQ ID NO: 296 - protein, 4 aa, Homo sapiens
DIVV

SEQ ID NO: 297 - protein, 5 aa, Homo sapiens
RILYY

SEQ ID NO: 298 - protein, 5 aa, Homo sapiens
GYCSS

SEQ ID NO: 299 - protein, 5 aa, Homo sapiens
DIVVV

SEQ ID NO: 300 - protein, 6 aa, Homo sapiens
RILYYY

SEQ ID NO: 301 - protein, 6 aa, Homo sapiens
GYCSST

SEQ ID NO: 302 - protein, 6 aa, Homo sapiens
DIVVVP

SEQ ID NO: 303 - protein, 4 aa, Homo sapiens
ILYY

SEQ ID NO: 304 - protein, 4 aa, Homo sapiens
YCSS

SEQ ID NO: 305 - protein, 4 aa, Homo sapiens
IVVV

SEQ ID NO: 306 - protein, 5 aa, Homo sapiens
ILYYY

SEQ ID NO: 307 - protein, 5 aa, Homo sapiens
YCSST

SEQ ID NO: 308 - protein, 5 aa, Homo sapiens
IVVVP

SEQ ID NO: 309 - protein, 6 aa, Homo sapiens
ILYYYQ

SEQ ID NO: 310 - protein, 6 aa, Homo sapiens
YCSSTS

SEQ ID NO: 311 - protein, 6 aa, Homo sapiens
IVVVPA

SEQ ID NO: 312 - protein, 4 aa, Homo sapiens
LYYY

SEQ ID NO: 313 - protein, 4 aa, Homo sapiens
CSST

SEQ ID NO: 314 - protein, 4 aa, Homo sapiens
VVVP

SEQ ID NO: 315 - protein, 5 aa, Homo sapiens
LYYYQ

SEQ ID NO: 316 - protein, 5 aa, Homo sapiens
CSSTS

SEQ ID NO: 317 - protein, 5 aa, Homo sapiens
VVVPA

SEQ ID NO: 318 - protein, 6 aa, Homo sapiens
LYYYQL

SEQ ID NO: 319 - protein, 6 aa, Homo sapiens
CSSTSC

SEQ ID NO: 320 - protein, 6 aa, Homo sapiens
VVVPAA

SEQ ID NO: 321 - protein, 4 aa, Homo sapiens
YYYQ

SEQ ID NO: 322 - protein, 4 aa, Homo sapiens
SSTS

SEQ ID NO: 323 - protein, 4 aa, Homo sapiens
VVPA

SEQ ID NO: 324 - protein, 5 aa, Homo sapiens
YYYQL

SEQ ID NO: 325 - protein, 5 aa, Homo sapiens
SSTSC

SEQ ID NO: 326 - protein, 5 aa, Homo sapiens
VVPAA

SEQ ID NO: 327 - protein, 6 aa, Homo sapiens
YYYQLL

SEQ ID NO: 328 - protein, 6 aa, Homo sapiens
SSTSCY

SEQ ID NO: 329 - protein, 6 aa, Homo sapiens
VVPAAI

SEQ ID NO: 330 - protein, 4 aa, Homo sapiens
YYQL

SEQ ID NO: 331 - protein, 4 aa, Homo sapiens
STSC

SEQ ID NO: 332 - protein, 4 aa, Homo sapiens
VPAA

SEQ ID NO: 333 - protein, 5 aa, Homo sapiens
YYQLL

SEQ ID NO: 334 - protein, 5 aa, Homo sapiens
STSCY

SEQ ID NO: 335 - protein, 5 aa, Homo sapiens
VPAAI

SEQ ID NO: 336 - protein, 6 aa, Homo sapiens
YYQLLY

SEQ ID NO: 337 - protein, 6 aa, Homo sapiens
STSCYT

SEQ ID NO: 338 - protein, 4 aa, Homo sapiens
YQLL

SEQ ID NO: 339 - protein, 4 aa, Homo sapiens
TSCY

SEQ ID NO: 340 - protein, 4 aa, Homo sapiens
PAAI

SEQ ID NO: 341 - protein, 5 aa, Homo sapiens
YQLLY

SEQ ID NO: 342 - protein, 5 aa, Homo sapiens
TSCYT

SEQ ID NO: 343 - protein, 4 aa, Homo sapiens
QLLY

SEQ ID NO: 344 - protein, 4 aa, Homo sapiens
SCYT

SEQ ID NO: 345 - protein, 4 aa, Homo sapiens
RILY

SEQ ID NO: 346 - protein, 4 aa, Homo sapiens
GYCT

SEQ ID NO: 347 - protein, 4 aa, Homo sapiens
DIVL

SEQ ID NO: 348 - protein, 5 aa, Homo sapiens
RILYY

SEQ ID NO: 349 - protein, 5 aa, Homo sapiens
GYCTN

SEQ ID NO: 350 - protein, 5 aa, Homo sapiens
DIVLM

SEQ ID NO: 351 - protein, 6 aa, Homo sapiens
RILYYW

SEQ ID NO: 352 - protein, 6 aa, Homo sapiens
GYCTNG

SEQ ID NO: 353 - protein, 6 aa, Homo sapiens
DIVLMV

SEQ ID NO: 354 - protein, 4 aa, Homo sapiens
ILYY

SEQ ID NO: 355 - protein, 4 aa, Homo sapiens
YCTN

SEQ ID NO: 356 - protein, 4 aa, Homo sapiens
IVLM

SEQ ID NO: 357 - protein, 5 aa, Homo sapiens
ILYYW

SEQ ID NO: 358 - protein, 5 aa, Homo sapiens
YCTNG

SEQ ID NO: 359 - protein, 5 aa, Homo sapiens
IVLMV

SEQ ID NO: 360 - protein, 6 aa, Homo sapiens
ILYYWC

SEQ ID NO: 361 - protein, 6 aa, Homo sapiens
YCTNGV

SEQ ID NO: 362 - protein, 6 aa, Homo sapiens
IVLMVY

SEQ ID NO: 363 - protein, 4 aa, Homo sapiens
LYYW

SEQ ID NO: 364 - protein, 4 aa, Homo sapiens
CTNG

SEQ ID NO: 365 - protein, 4 aa, Homo sapiens
VLMV

SEQ ID NO: 366 - protein, 5 aa, Homo sapiens
LYYWC

SEQ ID NO: 367 - protein, 5 aa, Homo sapiens
CTNGV

SEQ ID NO: 368 - protein, 5 aa, Homo sapiens
VLMVY

SEQ ID NO: 369 - protein, 6 aa, Homo sapiens
LYYWCM

SEQ ID NO: 370 - protein, 6 aa, Homo sapiens
CTNGVC

SEQ ID NO: 371 - protein, 6 aa, Homo sapiens
VLMVYA

SEQ ID NO: 372 - protein, 4 aa, Homo sapiens
YYWC

SEQ ID NO: 373 - protein, 4 aa, Homo sapiens
TNGV

SEQ ID NO: 374 - protein, 4 aa, Homo sapiens
LMVY

SEQ ID NO: 375 - protein, 5 aa, Homo sapiens
YYWCM

SEQ ID NO: 376 - protein, 5 aa, Homo sapiens
TNGVC

SEQ ID NO: 377 - protein, 5 aa, Homo sapiens
LMVYA

SEQ ID NO: 378 - protein, 6 aa, Homo sapiens
YYWCML

SEQ ID NO: 379 - protein, 6 aa, Homo sapiens
TNGVCY

SEQ ID NO: 380 - protein, 6 aa, Homo sapiens
LMVYAI

SEQ ID NO: 381 - protein, 4 aa, Homo sapiens
YWCM

SEQ ID NO: 382 - protein, 4 aa, Homo sapiens
NGVC

SEQ ID NO: 383 - protein, 4 aa, Homo sapiens
MVYA

SEQ ID NO: 384 - protein, 5 aa, Homo sapiens
YWCML

SEQ ID NO: 385 - protein, 5 aa, Homo sapiens
NGVCY

SEQ ID NO: 386 - protein, 5 aa, Homo sapiens
MVYAI

SEQ ID NO: 387 - protein, 6 aa, Homo sapiens
YWCMLY

SEQ ID NO: 388 - protein, 6 aa, Homo sapiens
NGVCYT

SEQ ID NO: 389 - protein, 4 aa, Homo sapiens
WCML

SEQ ID NO: 390 - protein, 4 aa, Homo sapiens
GVCY

SEQ ID NO: 391 - protein, 4 aa, Homo sapiens
VYAI

SEQ ID NO: 392 - protein, 5 aa, Homo sapiens
WCMLY

SEQ ID NO: 393 - protein, 5 aa, Homo sapiens
GVCYT

SEQ ID NO: 394 - protein, 4 aa, Homo sapiens
CMLY

SEQ ID NO: 395 - protein, 4 aa, Homo sapiens
VCYT

SEQ ID NO: 396 - protein, 5 aa, Homo sapiens
RILYW

SEQ ID NO: 397 - protein, 5 aa, Homo sapiens
GYCSG

SEQ ID NO: 398 - protein, 6 aa, Homo sapiens
RILYWW

SEQ ID NO: 399 - protein, 6 aa, Homo sapiens
GYCSGG

SEQ ID NO: 400 - protein, 6 aa, Homo sapiens
DIVVVV

SEQ ID NO: 401 - protein, 4 aa, Homo sapiens
ILYW

SEQ ID NO: 402 - protein, 4 aa, Homo sapiens
YCSG

SEQ ID NO: 403 - protein, 5 aa, Homo sapiens
ILYWW

SEQ ID NO: 404 - protein, 5 aa, Homo sapiens
YCSGG

SEQ ID NO: 405 - protein, 5 aa, Homo sapiens
IVVVV

SEQ ID NO: 406 - protein, 6 aa, Homo sapiens
ILYWWY

SEQ ID NO: 407 - protein, 6 aa, Homo sapiens
YCSGGS

SEQ ID NO: 408 - protein, 6 aa, Homo sapiens
IVVVVA

SEQ ID NO: 409 - protein, 4 aa, Homo sapiens
LYWW

SEQ ID NO: 410 - protein, 4 aa, Homo sapiens
CSGG

SEQ ID NO: 411 - protein, 4 aa, Homo sapiens
VVVV

SEQ ID NO: 412 - protein, 5 aa, Homo sapiens
LYWWY

SEQ ID NO: 413 - protein, 5 aa, Homo sapiens
CSGGS

SEQ ID NO: 414 - protein, 5 aa, Homo sapiens
VVVVA

SEQ ID NO: 415 - protein, 6 aa, Homo sapiens
LYWWYL

SEQ ID NO: 416 - protein, 6 aa, Homo sapiens
CSGGSC

SEQ ID NO: 417 - protein, 6 aa, Homo sapiens
VVVVAA

SEQ ID NO: 418 - protein, 4 aa, Homo sapiens
YWWY

SEQ ID NO: 419 - protein, 4 aa, Homo sapiens
SGGS

SEQ ID NO: 420 - protein, 4 aa, Homo sapiens
VVVA

SEQ ID NO: 421 - protein, 5 aa, Homo sapiens
YWWYL

SEQ ID NO: 422 - protein, 5 aa, Homo sapiens
SGGSC

SEQ ID NO: 423 - protein, 5 aa, Homo sapiens
VVVAA

SEQ ID NO: 424 - protein, 6 aa, Homo sapiens
YWWYLL

SEQ ID NO: 425 - protein, 6 aa, Homo sapiens
SGGSCY

SEQ ID NO: 426 - protein, 6 aa, Homo sapiens
VVVAAT

SEQ ID NO: 427 - protein, 4 aa, Homo sapiens
WWYL

SEQ ID NO: 428 - protein, 4 aa, Homo sapiens
GGSC

SEQ ID NO: 429 - protein, 4 aa, Homo sapiens
VVAA

SEQ ID NO: 430 - protein, 5 aa, Homo sapiens
WWYLL

SEQ ID NO: 431 - protein, 5 aa, Homo sapiens
GGSCY

SEQ ID NO: 432 - protein, 5 aa, Homo sapiens
VVAAT

SEQ ID NO: 433 - protein, 6 aa, Homo sapiens
WWYLLL

SEQ ID NO: 434 - protein, 6 aa, Homo sapiens
GGSCYS

SEQ ID NO: 435 - protein, 4 aa, Homo sapiens
WYLL

SEQ ID NO: 436 - protein, 4 aa, Homo sapiens
GSCY

SEQ ID NO: 437 - protein, 4 aa, Homo sapiens
VAAT

SEQ ID NO: 438 - protein, 5 aa, Homo sapiens
WYLLL

SEQ ID NO: 439 - protein, 5 aa, Homo sapiens
GSCYS

SEQ ID NO: 440 - protein, 4 aa, Homo sapiens
YLLL

SEQ ID NO: 441 - protein, 4 aa, Homo sapiens
SCYS

SEQ ID NO: 442 - protein, 4 aa, Homo sapiens
SILW

SEQ ID NO: 443 - protein, 4 aa, Homo sapiens
AYCG

SEQ ID NO: 444 - protein, 4 aa, Homo sapiens
HIVV

SEQ ID NO: 445 - protein, 5 aa, Homo sapiens
SILWW

SEQ ID NO: 446 - protein, 5 aa, Homo sapiens
AYCGG

SEQ ID NO: 447 - protein, 5 aa, Homo sapiens
HIVVV

SEQ ID NO: 448 - protein, 6 aa, Homo sapiens
SILWWW

SEQ ID NO: 449 - protein, 6 aa, Homo sapiens
AYCGGD

SEQ ID NO: 450 - protein, 6 aa, Homo sapiens
HIVVVT

SEQ ID NO: 451 - protein, 4 aa, Homo sapiens
ILWW

SEQ ID NO: 452 - protein, 4 aa, Homo sapiens
YCGG

SEQ ID NO: 453 - protein, 5 aa, Homo sapiens
ILWWW

SEQ ID NO: 454 - protein, 5 aa, Homo sapiens
YCGGD

SEQ ID NO: 455 - protein, 5 aa, Homo sapiens
IVVVT

SEQ ID NO: 456 - protein, 6 aa, Homo sapiens
ILWWWL

SEQ ID NO: 457 - protein, 6 aa, Homo sapiens
YCGGDC

SEQ ID NO: 458 - protein, 6 aa, Homo sapiens
IVVVTA

SEQ ID NO: 459 - protein, 4 aa, Homo sapiens
LWWW

SEQ ID NO: 460 - protein, 4 aa, Homo sapiens
CGGD

SEQ ID NO: 461 - protein, 4 aa, Homo sapiens
VVVT

SEQ ID NO: 462 - protein, 5 aa, Homo sapiens
LWWWL

SEQ ID NO: 463 - protein, 5 aa, Homo sapiens
CGGDC

SEQ ID NO: 464 - protein, 5 aa, Homo sapiens
VVVTA

SEQ ID NO: 465 - protein, 6 aa, Homo sapiens
LWWWLL

SEQ ID NO: 466 - protein, 6 aa, Homo sapiens
CGGDCY

SEQ ID NO: 467 - protein, 6 aa, Homo sapiens
VVVTAI

SEQ ID NO: 468 - protein, 4 aa, Homo sapiens
WWWL

SEQ ID NO: 469 - protein, 4 aa, Homo sapiens
GGDC

SEQ ID NO: 470 - protein, 4 aa, Homo sapiens
VVTA

SEQ ID NO: 471 - protein, 5 aa, Homo sapiens
WWWLL

SEQ ID NO: 472 - protein, 5 aa, Homo sapiens
GGDCY

SEQ ID NO: 473 - protein, 5 aa, Homo sapiens
VVTAI

SEQ ID NO: 474 - protein, 6 aa, Homo sapiens
WWWLLF

SEQ ID NO: 475 - protein, 6 aa, Homo sapiens
GGDCYS

SEQ ID NO: 476 - protein, 4 aa, Homo sapiens
WWLL

SEQ ID NO: 477 - protein, 4 aa, Homo sapiens
GDCY

SEQ ID NO: 478 - protein, 4 aa, Homo sapiens
VTAI

SEQ ID NO: 479 - protein, 5 aa, Homo sapiens
WWLLF

SEQ ID NO: 480 - protein, 5 aa, Homo sapiens
GDCYS

SEQ ID NO: 481 - protein, 4 aa, Homo sapiens
WLLF

SEQ ID NO: 482 - protein, 4 aa, Homo sapiens
DCYS

SEQ ID NO: 483 - protein, 4 aa, Homo sapiens
VLRF

SEQ ID NO: 484 - protein, 4 aa, Homo sapiens
YYDF

SEQ ID NO: 485 - protein, 4 aa, Homo sapiens
ITIF

SEQ ID NO: 486 - protein, 5 aa, Homo sapiens
VLRFL

SEQ ID NO: 487 - protein, 5 aa, Homo sapiens
YYDFW

SEQ ID NO: 488 - protein, 5 aa, Homo sapiens
ITIFG

SEQ ID NO: 489 - protein, 6 aa, Homo sapiens
VLRFLE

SEQ ID NO: 490 - protein, 6 aa, Homo sapiens
YYDFWS

SEQ ID NO: 491 - protein, 6 aa, Homo sapiens
ITIFGV

SEQ ID NO: 492 - protein, 4 aa, Homo sapiens
LRFL

SEQ ID NO: 493 - protein, 4 aa, Homo sapiens
YDFW

SEQ ID NO: 494 - protein, 4 aa, Homo sapiens
TIFG

SEQ ID NO: 495 - protein, 5 aa, Homo sapiens
LRFLE

SEQ ID NO: 496 - protein, 5 aa, Homo sapiens
YDFWS

SEQ ID NO: 497 - protein, 5 aa, Homo sapiens
TIFGV

SEQ ID NO: 498 - protein, 6 aa, Homo sapiens
LRFLEW

SEQ ID NO: 499 - protein, 6 aa, Homo sapiens
YDFWSG

SEQ ID NO: 500 - protein, 6 aa, Homo sapiens
TIFGVV

SEQ ID NO: 501 - protein, 4 aa, Homo sapiens
RFLE

SEQ ID NO: 502 - protein, 4 aa, Homo sapiens
DFWS

SEQ ID NO: 503 - protein, 4 aa, Homo sapiens
IFGV

SEQ ID NO: 504 - protein, 5 aa, Homo sapiens
RFLEW

SEQ ID NO: 505 - protein, 5 aa, Homo sapiens
DFWSG

SEQ ID NO: 506 - protein, 5 aa, Homo sapiens
IFGVV

SEQ ID NO: 507 - protein, 6 aa, Homo sapiens
RFLEWL

SEQ ID NO: 508 - protein, 6 aa, Homo sapiens
DFWSGY

SEQ ID NO: 509 - protein, 6 aa, Homo sapiens
IFGVVI

SEQ ID NO: 510 - protein, 4 aa, Homo sapiens
FLEW

SEQ ID NO: 511 - protein, 4 aa, Homo sapiens
FWSG

SEQ ID NO: 512 - protein, 4 aa, Homo sapiens
FGVV

SEQ ID NO: 513 - protein, 5 aa, Homo sapiens
FLEWL

SEQ ID NO: 514 - protein, 5 aa, Homo sapiens
FWSGY

SEQ ID NO: 515 - protein, 5 aa, Homo sapiens
FGVVI

SEQ ID NO: 516 - protein, 6 aa, Homo sapiens
FLEWLL

SEQ ID NO: 517 - protein, 6 aa, Homo sapiens
FWSGYY

SEQ ID NO: 518 - protein, 6 aa, Homo sapiens
FGVVII

SEQ ID NO: 519 - protein, 4 aa, Homo sapiens
LEWL

SEQ ID NO: 520 - protein, 4 aa, Homo sapiens
WSGY

SEQ ID NO: 521 - protein, 4 aa, Homo sapiens
GVVI

SEQ ID NO: 522 - protein, 5 aa, Homo sapiens
LEWLL

SEQ ID NO: 523 - protein, 5 aa, Homo sapiens
WSGYY

SEQ ID NO: 524 - protein, 5 aa, Homo sapiens
GVVII

SEQ ID NO: 525 - protein, 6 aa, Homo sapiens
LEWLLY

SEQ ID NO: 526 - protein, 6 aa, Homo sapiens
WSGYYT

SEQ ID NO: 527 - protein, 4 aa, Homo sapiens
EWLL

SEQ ID NO: 528 - protein, 4 aa, Homo sapiens
SGYY

SEQ ID NO: 529 - protein, 4 aa, Homo sapiens
VVII

SEQ ID NO: 530 - protein, 5 aa, Homo sapiens
EWLLY

SEQ ID NO: 531 - protein, 5 aa, Homo sapiens
SGYYT

SEQ ID NO: 532 - protein, 4 aa, Homo sapiens
WLLY

SEQ ID NO: 533 - protein, 4 aa, Homo sapiens
GYYT

SEQ ID NO: 534 - protein, 4 aa, Homo sapiens
VLRY

SEQ ID NO: 535 - protein, 4 aa, Homo sapiens
YYDI

SEQ ID NO: 536 - protein, 5 aa, Homo sapiens
VLRYF

SEQ ID NO: 537 - protein, 5 aa, Homo sapiens
YYDIL

SEQ ID NO: 538 - protein, 5 aa, Homo sapiens
ITIFY

SEQ ID NO: 539 - protein, 6 aa, Homo sapiens
VLRYFD

SEQ ID NO: 540 - protein, 6 aa, Homo sapiens
YYDILT

SEQ ID NO: 541 - protein, 6 aa, Homo sapiens
ITIFYL

SEQ ID NO: 542 - protein, 4 aa, Homo sapiens
LRYF

SEQ ID NO: 543 - protein, 4 aa, Homo sapiens
YDIL

SEQ ID NO: 544 - protein, 4 aa, Homo sapiens
TIFY

SEQ ID NO: 545 - protein, 5 aa, Homo sapiens
LRYFD

SEQ ID NO: 546 - protein, 5 aa, Homo sapiens
YDILT

SEQ ID NO: 547 - protein, 5 aa, Homo sapiens
TIFYL

SEQ ID NO: 548 - protein, 6 aa, Homo sapiens
LRYFDW

SEQ ID NO: 549 - protein, 6 aa, Homo sapiens
YDILTG

SEQ ID NO: 550 - protein, 6 aa, Homo sapiens
TIFYLV

SEQ ID NO: 551 - protein, 4 aa, Homo sapiens
RYFD

SEQ ID NO: 552 - protein, 4 aa, Homo sapiens
DILT

SEQ ID NO: 553 - protein, 4 aa, Homo sapiens
IFYL

SEQ ID NO: 554 - protein, 5 aa, Homo sapiens
RYFDW

SEQ ID NO: 555 - protein, 5 aa, Homo sapiens
DILTG

SEQ ID NO: 556 - protein, 5 aa, Homo sapiens
IFYLV

SEQ ID NO: 557 - protein, 6 aa, Homo sapiens
RYFDWL

SEQ ID NO: 558 - protein, 6 aa, Homo sapiens
DILTGY

SEQ ID NO: 559 - protein, 6 aa, Homo sapiens
IFYLVI

SEQ ID NO: 560 - protein, 4 aa, Homo sapiens
YFDW

SEQ ID NO: 561 - protein, 4 aa, Homo sapiens
ILTG

SEQ ID NO: 562 - protein, 4 aa, Homo sapiens
FYLV

SEQ ID NO: 563 - protein, 5 aa, Homo sapiens
YFDWL

SEQ ID NO: 564 - protein, 5 aa, Homo sapiens
ILTGY

SEQ ID NO: 565 - protein, 5 aa, Homo sapiens
FYLVI

SEQ ID NO: 566 - protein, 6 aa, Homo sapiens
YFDWLL

SEQ ID NO: 567 - protein, 6 aa, Homo sapiens
ILTGYY

SEQ ID NO: 568 - protein, 6 aa, Homo sapiens
FYLVII

SEQ ID NO: 569 - protein, 4 aa, Homo sapiens
FDWL

SEQ ID NO: 570 - protein, 4 aa, Homo sapiens
LTGY

SEQ ID NO: 571 - protein, 4 aa, Homo sapiens
YLVI

SEQ ID NO: 572 - protein, 5 aa, Homo sapiens
FDWLL

SEQ ID NO: 573 - protein, 5 aa, Homo sapiens
LTGYY

SEQ ID NO: 574 - protein, 5 aa, Homo sapiens
YLVII

SEQ ID NO: 575 - protein, 6 aa, Homo sapiens
FDWLLY

SEQ ID NO: 576 - protein, 6 aa, Homo sapiens
LTGYYN

SEQ ID NO: 577 - protein, 4 aa, Homo sapiens
DWLL

SEQ ID NO: 578 - protein, 4 aa, Homo sapiens
TGYY

SEQ ID NO: 579 - protein, 4 aa, Homo sapiens
LVII

SEQ ID NO: 580 - protein, 5 aa, Homo sapiens
DWLLY

SEQ ID NO: 581 - protein, 5 aa, Homo sapiens
TGYYN

SEQ ID NO: 582 - protein, 4 aa, Homo sapiens
WLLY

SEQ ID NO: 583 - protein, 4 aa, Homo sapiens
GYYN

SEQ ID NO: 584 - protein, 4 aa, Homo sapiens
VLLW

SEQ ID NO: 585 - protein, 4 aa, Homo sapiens
YYYG

SEQ ID NO: 586 - protein, 4 aa, Homo sapiens
ITMV

SEQ ID NO: 587 - protein, 5 aa, Homo sapiens
VLLWF

SEQ ID NO: 588 - protein, 5 aa, Homo sapiens
YYYGS

SEQ ID NO: 589 - protein, 5 aa, Homo sapiens
ITMVR

SEQ ID NO: 590 - protein, 6 aa, Homo sapiens
VLLWFG

SEQ ID NO: 591 - protein, 6 aa, Homo sapiens
YYYGSG

SEQ ID NO: 592 - protein, 6 aa, Homo sapiens
ITMVRG

SEQ ID NO: 593 - protein, 4 aa, Homo sapiens
LLWF

SEQ ID NO: 594 - protein, 4 aa, Homo sapiens
YYGS

SEQ ID NO: 595 - protein, 4 aa, Homo sapiens
TMVR

SEQ ID NO: 596 - protein, 5 aa, Homo sapiens
LLWFG

SEQ ID NO: 597 - protein, 5 aa, Homo sapiens
YYGSG

SEQ ID NO: 598 - protein, 5 aa, Homo sapiens
TMVRG

SEQ ID NO: 599 - protein, 6 aa, Homo sapiens
LLWFGE

SEQ ID NO: 600 - protein, 6 aa, Homo sapiens
YYGSGS

SEQ ID NO: 601 - protein, 6 aa, Homo sapiens
TMVRGV

SEQ ID NO: 602 - protein, 4 aa, Homo sapiens
LWFG

SEQ ID NO: 603 - protein, 4 aa, Homo sapiens
YGSG

SEQ ID NO: 604 - protein, 4 aa, Homo sapiens
MVRG

SEQ ID NO: 605 - protein, 5 aa, Homo sapiens
LWFGE

SEQ ID NO: 606 - protein, 5 aa, Homo sapiens
YGSGS

SEQ ID NO: 607 - protein, 5 aa, Homo sapiens
MVRGV

SEQ ID NO: 608 - protein, 6 aa, Homo sapiens
LWFGEL

SEQ ID NO: 609 - protein, 6 aa, Homo sapiens
YGSGSY

SEQ ID NO: 610 - protein, 6 aa, Homo sapiens
MVRGVI

SEQ ID NO: 611 - protein, 4 aa, Homo sapiens
WFGE

SEQ ID NO: 612 - protein, 4 aa, Homo sapiens
GSGS

SEQ ID NO: 613 - protein, 4 aa, Homo sapiens
VRGV

SEQ ID NO: 614 - protein, 5 aa, Homo sapiens
WFGEL

SEQ ID NO: 615 - protein, 5 aa, Homo sapiens
GSGSY

SEQ ID NO: 616 - protein, 5 aa, Homo sapiens
VRGVI

SEQ ID NO: 617 - protein, 6 aa, Homo sapiens
WFGELL

SEQ ID NO: 618 - protein, 6 aa, Homo sapiens
GSGSYY

SEQ ID NO: 619 - protein, 6 aa, Homo sapiens
VRGVII

SEQ ID NO: 620 - protein, 4 aa, Homo sapiens
FGEL

SEQ ID NO: 621 - protein, 4 aa, Homo sapiens
RGVI

SEQ ID NO: 622 - protein, 5 aa, Homo sapiens
FGELL

SEQ ID NO: 623 - protein, 5 aa, Homo sapiens
RGVII

SEQ ID NO: 624 - protein, 6 aa, Homo sapiens
FGELLY

SEQ ID NO: 625 - protein, 6 aa, Homo sapiens
SGSYYN

SEQ ID NO: 626 - protein, 4 aa, Homo sapiens
GELL

SEQ ID NO: 627 - protein, 4 aa, Homo sapiens
GVII

SEQ ID NO: 628 - protein, 5 aa, Homo sapiens
GELLY

SEQ ID NO: 629 - protein, 5 aa, Homo sapiens
GSYYN

SEQ ID NO: 630 - protein, 4 aa, Homo sapiens
ELLY

SEQ ID NO: 631 - protein, 4 aa, Homo sapiens
SYYN

SEQ ID NO: 632 - protein, 4 aa, Homo sapiens
VLWL

SEQ ID NO: 633 - protein, 4 aa, Homo sapiens
YYDY

SEQ ID NO: 634 - protein, 4 aa, Homo sapiens
IMIT

SEQ ID NO: 635 - protein, 5 aa, Homo sapiens
VLWLR

SEQ ID NO: 636 - protein, 5 aa, Homo sapiens
YYDYV

SEQ ID NO: 637 - protein, 5 aa, Homo sapiens
IMITF

SEQ ID NO: 638 - protein, 6 aa, Homo sapiens
VLWLRL

SEQ ID NO: 639 - protein, 6 aa, Homo sapiens
YYDYVW

SEQ ID NO: 640 - protein, 6 aa, Homo sapiens
IMITFG

SEQ ID NO: 641 - protein, 4 aa, Homo sapiens
LWLR

SEQ ID NO: 642 - protein, 4 aa, Homo sapiens
YDYV

SEQ ID NO: 643 - protein, 4 aa, Homo sapiens
MITF

SEQ ID NO: 644 - protein, 5 aa, Homo sapiens
LWLRL

SEQ ID NO: 645 - protein, 5 aa, Homo sapiens
YDYVW

SEQ ID NO: 646 - protein, 5 aa, Homo sapiens
MITFG

SEQ ID NO: 647 - protein, 6 aa, Homo sapiens
LWLRLG

SEQ ID NO: 648 - protein, 6 aa, Homo sapiens
YDYVWG

SEQ ID NO: 649 - protein, 6 aa, Homo sapiens
MITFGG

SEQ ID NO: 650 - protein, 4 aa, Homo sapiens
WLRL

SEQ ID NO: 651 - protein, 4 aa, Homo sapiens
DYVW

SEQ ID NO: 652 - protein, 4 aa, Homo sapiens
ITFG

SEQ ID NO: 653 - protein, 5 aa, Homo sapiens
WLRLG

SEQ ID NO: 654 - protein, 5 aa, Homo sapiens
DYVWG

SEQ ID NO: 655 - protein, 5 aa, Homo sapiens
ITFGG

SEQ ID NO: 656 - protein, 6 aa, Homo sapiens
WLRLGE

SEQ ID NO: 657 - protein, 6 aa, Homo sapiens
DYVWGS

SEQ ID NO: 658 - protein, 6 aa, Homo sapiens
ITFGGV

SEQ ID NO: 659 - protein, 4 aa, Homo sapiens
LRLG

SEQ ID NO: 660 - protein, 4 aa, Homo sapiens
YVWG

SEQ ID NO: 661 - protein, 4 aa, Homo sapiens
TFGG

SEQ ID NO: 662 - protein, 5 aa, Homo sapiens
LRLGE

SEQ ID NO: 663 - protein, 5 aa, Homo sapiens
YVWGS

SEQ ID NO: 664 - protein, 5 aa, Homo sapiens
TFGGV

SEQ ID NO: 665 - protein, 6 aa, Homo sapiens
LRLGEL

SEQ ID NO: 666 - protein, 6 aa, Homo sapiens
YVWGSY

SEQ ID NO: 667 - protein, 6 aa, Homo sapiens
TFGGVI

SEQ ID NO: 668 - protein, 4 aa, Homo sapiens
RLGE

SEQ ID NO: 669 - protein, 4 aa, Homo sapiens
VWGS

SEQ ID NO: 670 - protein, 4 aa, Homo sapiens
FGGV

SEQ ID NO: 671 - protein, 5 aa, Homo sapiens
RLGEL

SEQ ID NO: 672 - protein, 5 aa, Homo sapiens
VWGSY

SEQ ID NO: 673 - protein, 5 aa, Homo sapiens
FGGVI

SEQ ID NO: 674 - protein, 6 aa, Homo sapiens
RLGELS

SEQ ID NO: 675 - protein, 6 aa, Homo sapiens
VWGSYR

SEQ ID NO: 676 - protein, 6 aa, Homo sapiens
FGGVIV

SEQ ID NO: 677 - protein, 4 aa, Homo sapiens
LGEL

SEQ ID NO: 678 - protein, 4 aa, Homo sapiens
WGSY

SEQ ID NO: 679 - protein, 4 aa, Homo sapiens
GGVI

SEQ ID NO: 680 - protein, 5 aa, Homo sapiens
LGELS

SEQ ID NO: 681 - protein, 5 aa, Homo sapiens
WGSYR

SEQ ID NO: 682 - protein, 5 aa, Homo sapiens
GGVIV

SEQ ID NO: 683 - protein, 6 aa, Homo sapiens
LGELSL

SEQ ID NO: 684 - protein, 6 aa, Homo sapiens
WGSYRY

SEQ ID NO: 685 - protein, 6 aa, Homo sapiens
GGVIVI

SEQ ID NO: 686 - protein, 4 aa, Homo sapiens
GELS

SEQ ID NO: 687 - protein, 4 aa, Homo sapiens
GSYR

SEQ ID NO: 688 - protein, 4 aa, Homo sapiens
GVIV

SEQ ID NO: 689 - protein, 5 aa, Homo sapiens
GELSL

SEQ ID NO: 690 - protein, 5 aa, Homo sapiens
GSYRY

SEQ ID NO: 691 - protein, 5 aa, Homo sapiens
GVIVI

SEQ ID NO: 692 - protein, 6 aa, Homo sapiens
GELSLY

SEQ ID NO: 693 - protein, 6 aa, Homo sapiens
GSYRYT

SEQ ID NO: 694 - protein, 4 aa, Homo sapiens
ELSL

SEQ ID NO: 695 - protein, 4 aa, Homo sapiens
SYRY

SEQ ID NO: 696 - protein, 4 aa, Homo sapiens
VIVI

SEQ ID NO: 697 - protein, 5 aa, Homo sapiens
ELSLY

SEQ ID NO: 698 - protein, 5 aa, Homo sapiens
SYRYT

SEQ ID NO: 699 - protein, 4 aa, Homo sapiens
LSLY

SEQ ID NO: 700 - protein, 4 aa, Homo sapiens
YRYT

SEQ ID NO: 701 - protein, 4 aa, Homo sapiens
VLLW

SEQ ID NO: 702 - protein, 4 aa, Homo sapiens
YYYD

SEQ ID NO: 703 - protein, 4 aa, Homo sapiens
ITMI

SEQ ID NO: 704 - protein, 5 aa, Homo sapiens
VLLWY

SEQ ID NO: 705 - protein, 5 aa, Homo sapiens
YYYDS

SEQ ID NO: 706 - protein, 5 aa, Homo sapiens
ITMIV

SEQ ID NO: 707 - protein, 6 aa, Homo sapiens
VLLWYY

SEQ ID NO: 708 - protein, 6 aa, Homo sapiens
YYYDSS

SEQ ID NO: 709 - protein, 6 aa, Homo sapiens
ITMIVV

SEQ ID NO: 710 - protein, 4 aa, Homo sapiens
LLWY

SEQ ID NO: 711 - protein, 4 aa, Homo sapiens
YYDS

SEQ ID NO: 712 - protein, 4 aa, Homo sapiens
TMIV

SEQ ID NO: 713 - protein, 5 aa, Homo sapiens
LLWYY

SEQ ID NO: 714 - protein, 5 aa, Homo sapiens
YYDSS

SEQ ID NO: 715 - protein, 5 aa, Homo sapiens
TMIVV

SEQ ID NO: 716 - protein, 6 aa, Homo sapiens
LLWYYW

SEQ ID NO: 717 - protein, 6 aa, Homo sapiens
YYDSSG

SEQ ID NO: 718 - protein, 6 aa, Homo sapiens
TMIVVV

SEQ ID NO: 719 - protein, 4 aa, Homo sapiens
LWYY

SEQ ID NO: 720 - protein, 4 aa, Homo sapiens
YDSS

SEQ ID NO: 721 - protein, 4 aa, Homo sapiens
MIVV

SEQ ID NO: 722 - protein, 5 aa, Homo sapiens
LWYYW

SEQ ID NO: 723 - protein, 5 aa, Homo sapiens
YDSSG

SEQ ID NO: 724 - protein, 5 aa, Homo sapiens
MIVVV

SEQ ID NO: 725 - protein, 6 aa, Homo sapiens
LWYYWL

SEQ ID NO: 726 - protein, 6 aa, Homo sapiens
YDSSGY

SEQ ID NO: 727 - protein, 6 aa, Homo sapiens
MIVVVI

SEQ ID NO: 728 - protein, 4 aa, Homo sapiens
WYYW

SEQ ID NO: 729 - protein, 4 aa, Homo sapiens
DSSG

SEQ ID NO: 730 - protein, 5 aa, Homo sapiens
WYYWL

SEQ ID NO: 731 - protein, 5 aa, Homo sapiens
DSSGY

SEQ ID NO: 732 - protein, 5 aa, Homo sapiens
IVVVI

SEQ ID NO: 733 - protein, 6 aa, Homo sapiens
WYYWLL

SEQ ID NO: 734 - protein, 6 aa, Homo sapiens
DSSGYY

SEQ ID NO: 735 - protein, 6 aa, Homo sapiens
IVVVIT

SEQ ID NO: 736 - protein, 4 aa, Homo sapiens
YYWL

SEQ ID NO: 737 - protein, 4 aa, Homo sapiens
SSGY

SEQ ID NO: 738 - protein, 4 aa, Homo sapiens
VVVI

SEQ ID NO: 739 - protein, 5 aa, Homo sapiens
YYWLL

SEQ ID NO: 740 - protein, 5 aa, Homo sapiens
SSGYY

SEQ ID NO: 741 - protein, 5 aa, Homo sapiens
VVVIT

SEQ ID NO: 742 - protein, 6 aa, Homo sapiens
YYWLLL

SEQ ID NO: 743 - protein, 6 aa, Homo sapiens
SSGYYY

SEQ ID NO: 744 - protein, 4 aa, Homo sapiens
YWLL

SEQ ID NO: 745 - protein, 4 aa, Homo sapiens
VVIT

SEQ ID NO: 746 - protein, 5 aa, Homo sapiens
YWLLL

SEQ ID NO: 747 - protein, 5 aa, Homo sapiens
SGYYY

SEQ ID NO: 748 - protein, 4 aa, Homo sapiens
WLLL

SEQ ID NO: 749 - protein, 4 aa, Homo sapiens
GYYY

SEQ ID NO: 750 - protein, 4 aa, Homo sapiens
WLQY

SEQ ID NO: 751 - protein, 4 aa, Homo sapiens
DYSN

SEQ ID NO: 752 - protein, 4 aa, Homo sapiens
TTVT

SEQ ID NO: 753 - protein, 5 aa, Homo sapiens
WLQYL

SEQ ID NO: 754 - protein, 5 aa, Homo sapiens
DYSNY

SEQ ID NO: 755 - protein, 4 aa, Homo sapiens
LQYL

SEQ ID NO: 756 - protein, 4 aa, Homo sapiens
YSNY

SEQ ID NO: 757 - protein, 4 aa, Homo sapiens
WLRW

SEQ ID NO: 758 - protein, 4 aa, Homo sapiens
DYGD

SEQ ID NO: 759 - protein, 5 aa, Homo sapiens
WLRWL

SEQ ID NO: 760 - protein, 5 aa, Homo sapiens
DYGDY

SEQ ID NO: 761 - protein, 4 aa, Homo sapiens
LRWL

SEQ ID NO: 762 - protein, 4 aa, Homo sapiens
YGDY

SEQ ID NO: 763 - protein, 4 aa, Homo sapiens
WLRW

SEQ ID NO: 764 - protein, 4 aa, Homo sapiens
DYGG

SEQ ID NO: 765 - protein, 4 aa, Homo sapiens
TTVV

SEQ ID NO: 766 - protein, 5 aa, Homo sapiens
WLRWY

SEQ ID NO: 767 - protein, 5 aa, Homo sapiens
DYGGN

SEQ ID NO: 768 - protein, 5 aa, Homo sapiens
TTVVT

SEQ ID NO: 769 - protein, 6 aa, Homo sapiens
WLRWYL

SEQ ID NO: 770 - protein, 6 aa, Homo sapiens
DYGGNS

SEQ ID NO: 771 - protein, 4 aa, Homo sapiens
LRWY

SEQ ID NO: 772 - protein, 4 aa, Homo sapiens
YGGN

SEQ ID NO: 773 - protein, 4 aa, Homo sapiens
TVVT

SEQ ID NO: 774 - protein, 5 aa, Homo sapiens
LRWYL

SEQ ID NO: 775 - protein, 5 aa, Homo sapiens
YGGNS

SEQ ID NO: 776 - protein, 4 aa, Homo sapiens
RWYL

SEQ ID NO: 777 - protein, 4 aa, Homo sapiens
GGNS

SEQ ID NO: 778 - protein, 4 aa, Homo sapiens
VDTA

SEQ ID NO: 779 - protein, 4 aa, Homo sapiens
WIQL

SEQ ID NO: 780 - protein, 4 aa, Homo sapiens
GYSY

SEQ ID NO: 781 - protein, 5 aa, Homo sapiens
VDTAM

SEQ ID NO: 782 - protein, 5 aa, Homo sapiens
WIQLW

SEQ ID NO: 783 - protein, 5 aa, Homo sapiens
GYSYG

SEQ ID NO: 784 - protein, 6 aa, Homo sapiens
VDTAMV

SEQ ID NO: 785 - protein, 6 aa, Homo sapiens
WIQLWL

SEQ ID NO: 786 - protein, 6 aa, Homo sapiens
GYSYGY

SEQ ID NO: 787 - protein, 4 aa, Homo sapiens
DTAM

SEQ ID NO: 788 - protein, 4 aa, Homo sapiens
IQLW

SEQ ID NO: 789 - protein, 4 aa, Homo sapiens
YSYG

SEQ ID NO: 790 - protein, 5 aa, Homo sapiens
DTAMV

SEQ ID NO: 791 - protein, 5 aa, Homo sapiens
IQLWL

SEQ ID NO: 792 - protein, 5 aa, Homo sapiens
YSYGY

SEQ ID NO: 793 - protein, 4 aa, Homo sapiens
TAMV

SEQ ID NO: 794 - protein, 4 aa, Homo sapiens
QLWL

SEQ ID NO: 795 - protein, 4 aa, Homo sapiens
SYGY

SEQ ID NO: 796 - protein, 4 aa, Homo sapiens
VDIV

SEQ ID NO: 797 - protein, 4 aa, Homo sapiens
WIYW

SEQ ID NO: 798 - protein, 4 aa, Homo sapiens
GYSG

SEQ ID NO: 799 - protein, 5 aa, Homo sapiens
VDIVA

SEQ ID NO: 800 - protein, 5 aa, Homo sapiens
WIYWL

SEQ ID NO: 801 - protein, 5 aa, Homo sapiens
GYSGY

SEQ ID NO: 802 - protein, 6 aa, Homo sapiens
VDIVAT

SEQ ID NO: 803 - protein, 6 aa, Homo sapiens
WIYWLR

SEQ ID NO: 804 - protein, 6 aa, Homo sapiens
GYSGYD

SEQ ID NO: 805 - protein, 4 aa, Homo sapiens
DIVA

SEQ ID NO: 806 - protein, 4 aa, Homo sapiens
IYWL

SEQ ID NO: 807 - protein, 4 aa, Homo sapiens
YSGY

SEQ ID NO: 808 - protein, 5 aa, Homo sapiens
DIVAT

SEQ ID NO: 809 - protein, 5 aa, Homo sapiens
IYWLR

SEQ ID NO: 810 - protein, 5 aa, Homo sapiens
YSGYD

SEQ ID NO: 811 - protein, 6 aa, Homo sapiens
DIVATI

SEQ ID NO: 812 - protein, 6 aa, Homo sapiens
IYWLRL

SEQ ID NO: 813 - protein, 6 aa, Homo sapiens
YSGYDY

SEQ ID NO: 814 - protein, 4 aa, Homo sapiens
IVAT

SEQ ID NO: 815 - protein, 4 aa, Homo sapiens
YWLR

SEQ ID NO: 816 - protein, 4 aa, Homo sapiens
SGYD

SEQ ID NO: 817 - protein, 5 aa, Homo sapiens
IVATI

SEQ ID NO: 818 - protein, 5 aa, Homo sapiens
YWLRL

SEQ ID NO: 819 - protein, 5 aa, Homo sapiens
SGYDY

SEQ ID NO: 820 - protein, 4 aa, Homo sapiens
VATI

SEQ ID NO: 821 - protein, 4 aa, Homo sapiens
WLRL

SEQ ID NO: 822 - protein, 4 aa, Homo sapiens
GYDY

SEQ ID NO: 823 - protein, 4 aa, Homo sapiens
VEMA

SEQ ID NO: 824 - protein, 4 aa, Homo sapiens
YRWL

SEQ ID NO: 825 - protein, 4 aa, Homo sapiens
RDGY

SEQ ID NO: 826 - protein, 5 aa, Homo sapiens
VEMAT

SEQ ID NO: 827 - protein, 5 aa, Homo sapiens
YRWLQ

SEQ ID NO: 828 - protein, 5 aa, Homo sapiens
RDGYN

SEQ ID NO: 829 - protein, 6 aa, Homo sapiens
VEMATI

SEQ ID NO: 830 - protein, 6 aa, Homo sapiens
YRWLQL

SEQ ID NO: 831 - protein, 6 aa, Homo sapiens
RDGYNY

SEQ ID NO: 832 - protein, 4 aa, Homo sapiens
EMAT

SEQ ID NO: 833 - protein, 4 aa, Homo sapiens
RWLQ

SEQ ID NO: 834 - protein, 4 aa, Homo sapiens
DGYN

SEQ ID NO: 835 - protein, 5 aa, Homo sapiens
EMATI

SEQ ID NO: 836 - protein, 5 aa, Homo sapiens
RWLQL

SEQ ID NO: 837 - protein, 5 aa, Homo sapiens
DGYNY

SEQ ID NO: 838 - protein, 4 aa, Homo sapiens
MATI

SEQ ID NO: 839 - protein, 4 aa, Homo sapiens
WLQL

SEQ ID NO: 840 - protein, 4 aa, Homo sapiens
GYNY

SEQ ID NO: 841 - protein, 4 aa, Homo sapiens
EYSS

SEQ ID NO: 842 - protein, 4 aa, Homo sapiens
SIAA

SEQ ID NO: 843 - protein, 4 aa, Homo sapiens
VYQL

SEQ ID NO: 844 - protein, 5 aa, Homo sapiens
EYSSS

SEQ ID NO: 845 - protein, 5 aa, Homo sapiens
SIAAR

SEQ ID NO: 846 - protein, 5 aa, Homo sapiens
VYQLV

SEQ ID NO: 847 - protein, 6 aa, Homo sapiens
EYSSSS

SEQ ID NO: 848 - protein, 4 aa, Homo sapiens
YSSS

SEQ ID NO: 849 - protein, 4 aa, Homo sapiens
IAAR

SEQ ID NO: 850 - protein, 4 aa, Homo sapiens
YQLV

SEQ ID NO: 851 - protein, 5 aa, Homo sapiens
YSSSS

SEQ ID NO: 852 - protein, 4 aa, Homo sapiens
SSSS

SEQ ID NO: 853 - protein, 4 aa, Homo sapiens
GYSS

SEQ ID NO: 854 - protein, 4 aa, Homo sapiens
GIAA

SEQ ID NO: 855 - protein, 4 aa, Homo sapiens
VYQQ

SEQ ID NO: 856 - protein, 5 aa, Homo sapiens
GYSSS

SEQ ID NO: 857 - protein, 5 aa, Homo sapiens
GIAAA

SEQ ID NO: 858 - protein, 5 aa, Homo sapiens
VYQQL

SEQ ID NO: 859 - protein, 6 aa, Homo sapiens
GYSSSW

SEQ ID NO: 860 - protein, 6 aa, Homo sapiens
GIAAAG

SEQ ID NO: 861 - protein, 6 aa, Homo sapiens
VYQQLV

SEQ ID NO: 862 - protein, 4 aa, Homo sapiens
IAAA

SEQ ID NO: 863 - protein, 4 aa, Homo sapiens
YQQL

SEQ ID NO: 864 - protein, 5 aa, Homo sapiens
YSSSW

SEQ ID NO: 865 - protein, 5 aa, Homo sapiens
IAAAG

SEQ ID NO: 866 - protein, 5 aa, Homo sapiens
YQQLV

SEQ ID NO: 867 - protein, 6 aa, Homo sapiens
YSSSWY

SEQ ID NO: 868 - protein, 4 aa, Homo sapiens
SSSW

SEQ ID NO: 869 - protein, 4 aa, Homo sapiens
AAAG

SEQ ID NO: 870 - protein, 4 aa, Homo sapiens
QQLV

SEQ ID NO: 871 - protein, 5 aa, Homo sapiens
SSSWY

SEQ ID NO: 872 - protein, 4 aa, Homo sapiens
SSWY

SEQ ID NO: 873 - protein, 4 aa, Homo sapiens
GIAV

SEQ ID NO: 874 - protein, 4 aa, Homo sapiens
VYQW

SEQ ID NO: 875 - protein, 5 aa, Homo sapiens
GYSSG

SEQ ID NO: 876 - protein, 5 aa, Homo sapiens
GIAVA

SEQ ID NO: 877 - protein, 5 aa, Homo sapiens
VYQWL

SEQ ID NO: 878 - protein, 6 aa, Homo sapiens
GYSSGW

SEQ ID NO: 879 - protein, 6 aa, Homo sapiens
GIAVAG

SEQ ID NO: 880 - protein, 6 aa, Homo sapiens
VYQWLV

SEQ ID NO: 881 - protein, 4 aa, Homo sapiens
YSSG

SEQ ID NO: 882 - protein, 4 aa, Homo sapiens
IAVA

SEQ ID NO: 883 - protein, 4 aa, Homo sapiens
YQWL

SEQ ID NO: 884 - protein, 5 aa, Homo sapiens
YSSGW

SEQ ID NO: 885 - protein, 5 aa, Homo sapiens
IAVAG

SEQ ID NO: 886 - protein, 5 aa, Homo sapiens
YQWLV

SEQ ID NO: 887 - protein, 6 aa, Homo sapiens
YSSGWY

SEQ ID NO: 888 - protein, 4 aa, Homo sapiens
SSGW

SEQ ID NO: 889 - protein, 4 aa, Homo sapiens
AVAG

SEQ ID NO: 890 - protein, 4 aa, Homo sapiens
QWLV

SEQ ID NO: 891 - protein, 5 aa, Homo sapiens
SSGWY

SEQ ID NO: 892 - protein, 432 aa, synthetic construct [sequence omitted]

SEQ ID NO: 893 - DNA, 1320 bp, synthetic construct
  misc_feature 1..1320: note = Synthetic polynucleotide
  source 1..1320: mol_type = other DNA
  CDS 1..1296
atgaaatacctattgcctacggcagccgctggattgttattactcgcggcccagccggccatggccgaagttcaattgttagagtctggtggcggtcttgttcagcctggtggttctttacgtctttcttgcgctgcttccggattcactttctcttcgtacgctatgtcttgggttcgccaagctcctggtaaaggtttggagtgggtttctgctatctctggttctggtggcagtacttactatgctgactccgttaaaggtcgcttcactatctctagagacaactctaagaatactctctacttgcagatgaacagcttaagggctgaggacactgcagtctactattgcgctaaagactatgaaggtactggttatgctttcgacatatggggtcaaggtactatggtcaccgtctctagtgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagttctgctagtaacgcgtcttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatgaaacgcgtgatgagaattc

SEQ ID NO: 894 - DNA, 9501 bp, synthetic construct
  misc_feature 1..9501: note = Synthetic polynucleotide
  source 1..9501: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacatgaaatacctattgcctacggcagccgctggattgttattactcgcggcccagccggccatggccgaagttcaattgttagagtctggtggcggtcttgttcagcctggtggttctttacgtctttcttgcgctgcttccggattcactttctcttcgtacgctatgtcttgggttcgccaagctcctggtaaaggtttggagtgggtttctgctatctctggttctggtggcagtacttactatgctgactccgttaaaggtcgcttcactatctctagagacaactctaagaatactctctacttgcagatgaacagcttaagggctgaggacactgcagtctactattgcgctaaagcctatcgtccttcttatcatgacatatggggtcaaggtactatggtcaccgtctctagtgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagctctgctagtggcgacttcgactacgagaaaatggctaatgccaacaaaggcgccatgactgagaacgctgacgagaatgctttgcaaagcgatgccaagggtaagttagacagcgtcgcgaccgactatggcgccgccatcgacggctttatcggcgatgtcagtggtttggccaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttcggtgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 895 - DNA, 5957 bp, synthetic construct
  misc_feature 1..5957: note = Synthetic polynucleotide
  source 1..5957: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacaggtccaactgcaggagctcgagatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgttaataaggcgcgcctaaccatctatttcaaggaacagtcttaatgaaaaagcttttattcatgatcccgttagttgtaccgttcgtggcccagccggcctctgctgaagttcaattgttagagtctggtggcggtcttgttcagcctggtggttctttacgtctttcttgcgctgcttccggagcttcagatctgtttgcctttttgtggggtggtgcagatcgcgttacggagatcgaccgactgcttgagcaaaagccacgcttaactgctgatcaggcatgggatgttattcgccaaaccagtcgtcaggatcttaacctgaggctttttttacctactctgcaagcagcgacatctggtttgacacagagcgatccgcgtcgtcagttggtagaaacattaacacgttgggatggcatcaatttgcttaatgatgatggtaaaacctggcagcagccaggctctgccatcctgaacgtttggctgaccagtatgttgaagcgtaccgtagtggctgccgtacctatgccatttgataagtggtacagcgccagtggctacgaaacaacccaggacggcccaactggttcgctgaatataagtgttggagcaaaaattttgtatgaggcggtgcagggagacaaatcaccaatcccacaggcggttgatctgtttgctgggaaaccacagcaggaggttgtgttggctgcgctggaagatacctgggagactctttccaaacgctatggcaataatgtgagtaactggaaaacaccggcaatggccttaacgttccgggcaaataatttctttggtgtaccgcaggccgcagcggaagaaacgcgtcatcaggcggagtatcaaaaccgtggaacagaaaacgatatgattgttttctcaccaacgacaagcgatcgtcctgtgcttgcctgggatgtggtcgcacccggtcagagtgggtttattgctcccgatggaacagttgataagcactatgaagatcagctgaaaatgtacgaaaattttggccgtaagtcgctctggttaacgaagcaggatgtggaggcgcataaggagttctagagacaactctaagaatactctctacttgcagatgaacagcttaagtctgagcattcggtccgggcaacattctccaaactgaccagacgacacaaacggcttacgctaaatcccgcgcatgggatggtaaagaggtggcgtctttgctggcctggactcatcagatgaaggccaaaaattggcaggagtggacacagcaggcagcgaaacaagcactgaccatcaactggtactatgctgatgtaaacggcaatattggttatgttcatactggtgcttatccagatcgtcaatcaggccatgatccgcgattacccgttcctggtacgggaaaatgggactggaaagggctattgccttttgaaatgaaccctaaggtgtataacccccagcagctagccatattctctcggtcaccgtctcaagcgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagttctgctagtaacgcgtcttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatgaaacgcgtgatgagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 896 - DNA, 3412 bp, synthetic construct
  misc_feature 1..3412: note = Synthetic polynucleotide
  source 1..3412: mol_type = other DNA
gacgaaagggcctgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaattgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccctgcaggtctcgggctattcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacatgaagaagctcctctttgctatcccgctcgtcgttccttttgtggcccagccggccatggccgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctttcactttcggccctgggaccaaagtggatatcaaacgtggtaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgtgcggccgctggtaagcctatccctaaccctctcctcggtctcgattctacgtgataacttcaccggtcaacgcgtgatgagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtctcagtacaatctgctctgatgccgcatagttaagccagccccgacacccgccaacacccgctgacgcgccctgacaggcttgtctgctcccggcatccgcttacagacaagctgtgaccgtctccgggagctgcatgtgtcagaggttttcaccgtcatcaccgaaacgcgcga

SEQ ID NO: 897 - protein, 232 aa, synthetic construct
  REGION 1..232: note = Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAAAGKPIPNPLLGLDST

SEQ ID NO: 898 - protein, 39 aa, synthetic construct
  REGION 1..39: note = Synthetic polypeptide
YYCAKDYGYCSSTSCYTKLYSYAEYFQHWGQGTLVTVSS

SEQ ID NO: 899 - protein, 39 aa, synthetic construct
  REGION 1..39: note = Synthetic polypeptide
YYCAKGSYYYGSGSYYNMDSYYAEYFQHWGQGTLVTVSS

SEQ ID NO: 900 - protein, 39 aa, synthetic construct
  REGION 1..39: note = Synthetic polypeptide
YYCAKEYYYYGSGSYYNSTTTSAEYFQHWGQGTLVTVSS

SEQ ID NO: 901 - protein, 51 aa, synthetic construct
  REGION 1..51: note = Synthetic polypeptide
YYCAKYYSFSYYPYYYDSSGYYYAYYSDYSYSYYAEYFQHWGQGTLVTVSS

SEQ ID NO: 902 - protein, 39 aa, synthetic construct
  REGION 1..39: note = Synthetic polypeptide
YYCAKSSGYCSSTSCYTNPYYYAEYFQHWGQGTLVTVSS

SEQ ID NO: 903 - protein, 51 aa, synthetic construct
  REGION 1..51: note = Synthetic polypeptide
YYCAKSYQYYGYCSSTSCYTYYSYWSYSSYYSYYAEYFQHWGQGTLVTVSS

SEQ ID NO: 904 - protein, 51 aa, synthetic construct
  REGION 1..51: note = Synthetic polypeptide
YYCAKYYSYYGYCSSTSCYTYSSSPSYSYYSSYYAEYFQHWGQGTLVTVSS

SEQ ID NO: 905 - protein, 50 aa, synthetic construct
  REGION 1..50: note = Synthetic polypeptide
YYCAKSPSYYDYVWGSYRYTSSYTYYSYSYSSYAEYFQHWGQGTLVTVSS

SEQ ID NO: 906 - protein, 50 aa, synthetic construct
  REGION 1..50: note = Synthetic polypeptide
YYCAKYAYSSESYYSSYYDYVWGSYRYTYSSYYAEYFQHWGQGTLVTVSS

SEQ ID NO: 907 - protein, 40 aa, synthetic construct
  REGION 1..40: note = Synthetic polypeptide
YYCAKGSSYYYGSGSYYNSEYYSAEYFQHWGQGTLVTVSS

SEQ ID NO: 908 - protein, 49 aa, synthetic construct
  REGION 1..49: note = Synthetic polypeptide
YYCARSSYYSYGYCTNGVCYTYSYSYYSYSYSYWYFDLWGRGTLVTVSS

SEQ ID NO: 909 - DNA, 18 bp, synthetic construct
  misc_feature 1..18: note = Synthetic oligonucleotide
  source 1..18: mol_type = other DNA
gcagtttattactgcgct

SEQ ID NO: 910 - DNA, 17 bp, synthetic construct
  misc_feature 1..17: note = Synthetic oligonucleotide
  source 1..17: mol_type = other DNA
agagtaccctggcccca

SEQ ID NO: 911 - DNA, 137 bp, synthetic construct
  misc_feature 1..137: note = Synthetic polynucleotide
  source 1..137: mol_type = other DNA
gcagtttattactgcgctaggtcttcctactattcctacggttattgtacaaatggcgtgtgctatacatactcctactcttattattcctattcttactcttactggtactttgatctgtggggccagggtactct

SEQ ID NO: 912 - protein, 50 aa, synthetic construct
  REGION 1..50: note = Synthetic polypeptide
YYCARSSSYYSYYSSGYCTNGVCYTYSSYYSSYYWYFDLWGRGTLVTVSS

SEQ ID NO: 913 - protein, 50 aa, synthetic construct
  REGION 1..50: note = Synthetic polypeptide
YYCAKYSYYSSSYYYSDYVWGSYRYTSYYSYYYAEYFQHWGQGTLVTVSS

SEQ ID NO: 914 - protein, 51 aa, synthetic construct
  REGION 1..51: note = Synthetic polypeptide
YYCAKSYYYSSYSYYYDYVWGSYRYTSYSSSSYYAEYFQHWGQGTLVTVSS

SEQ ID NO: 915 - protein, 51 aa, synthetic construct
  REGION 1..51: note = Synthetic polypeptide
YYCAKSSSYYSYSYSGYCSGGSCYSSYYYSSYYSAEYFQGWGQGTLVTVSS

SEQ ID NO: 916 - protein, 50 aa, synthetic construct
  REGION 1..50: note = Synthetic polypeptide
YYCAKYSSYSYYDYVWGSYRYTSSSYSYYSYYYAEYFQGWGQGTLVTVSS

SEQ ID NO: 917 - protein, 50 aa, synthetic construct
  REGION 1..50: note = Synthetic polypeptide
YYCAKSSYYYSSSYYDYVWGSYRYTSSYYSYSYAEYFQGWGQGTLVTVSS

SEQ ID NO: 918 - protein, 51 aa, synthetic construct
  REGION 1..51: note = Synthetic polypeptide
YYCAKYSSSSYSYYYYDSSGYYYSYYSSSYYSYYAEYFQGWGQGTLVTVSS

SEQ ID NO: 919 - protein, 49 aa, synthetic construct
  REGION 1..49: note = Synthetic polypeptide
YYCAKYSSYSSYYYYDSSGYYYSSYSSYSYYYAEYFQGWGQGTLVTVSS

SEQ ID NO: 920 - DNA, 97 bp, synthetic construct; identical to SEQ ID NO: 151
  misc_feature 1..97: note = Synthetic oligonucleotide
  source 1..97: mol_type = other DNA

SEQ ID NO: 921 - protein, 5 aa, synthetic construct
  REGION 1..5: note = Synthetic peptide
  VARIANT 5: note = K or R
YYCAX

SEQ ID NO: 922 - protein, 4 aa, synthetic construct
  REGION 1..4: note = Synthetic peptide
YYCA

SEQ ID NO: 923 - protein, 4 aa, synthetic construct
  REGION 1..4: note = Synthetic peptide
WGQG

SEQ ID NO: 924 - protein, 39 aa, synthetic construct
  REGION 1..39: note = Synthetic polypeptide
  VARIANT 5: note = K or R
  VARIANT 7: note = Y or S
  VARIANT 9..10: note = Y or S
  VARIANT 12: note = Y or S
  VARIANT 14: note = Y or S
  VARIANT 18: note = Y or S
  VARIANT 20: note = Y or S
  VARIANT 22: note = Y or S
  VARIANT 25: note = Y or S
YYCAXXXYXXGXGXXYNXXXYXAXXFQHWGQGTLVTVSS

SEQ ID NO: 925 - protein, 39 aa, synthetic construct
  REGION 1..39: note = Synthetic polypeptide
YYCARSSRSGYCTNGVCYTSKSYWYFDLWGRGTLVTVSS

SEQ ID NO: 926 - protein, 4 aa, Homo sapiens
SGWY

SEQ ID NO: 927 - protein, 4 aa, synthetic construct
  REGION 1..4: note = Synthetic peptide
YYSS

SEQ ID NO: 928 - protein, 6 aa, synthetic construct
  REGION 1..6: note = Synthetic peptide
YYSSYS

SEQ ID NO: 929 - protein, 8 aa, synthetic construct
  REGION 1..8: note = Synthetic peptide
SYYYSSYS

SEQ ID NO: 930 - protein, 10 aa, synthetic construct
  REGION 1..10: note = Synthetic peptide
SYYSYSSYYS

SEQ ID NO: 931 - protein, 4 aa, synthetic construct
  REGION 1..4: note = Synthetic peptide
SYSY

SEQ ID NO: 932 - protein, 4 aa, synthetic construct
  REGION 1..4: note = Synthetic peptide
YSSY

SEQ ID NO: 933 - protein, 4 aa, synthetic construct
  REGION 1..4: note = Synthetic peptide
SYYS

SEQ ID NO: 934 - DNA, 7423 bp, synthetic construct
  misc_feature 1..7423: note = Synthetic polynucleotide
  source 1..7423: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaac

SEQ ID NO: 935 - protein, 6 aa, synthetic construct
  REGION 1..6: note = Synthetic 6xHis tag
HHHHHH

SEQ ID NO: 936 - protein, 17 aa, synthetic construct
  REGION 1..17: note = Synthetic polypeptide
  VARIANT 2: note = Y or S
  VARIANT 4..5: note = Y or S
  VARIANT 7: note = Y or S
  VARIANT 9: note = Y or S
  VARIANT 13: note = Y or S
  VARIANT 15: note = Y or S
  VARIANT 17: note = Y or S
XXYXXGXGXXYNXXXYX

SEQ ID NO: 937 - protein, 29 aa, synthetic construct
  REGION 1..29: note = Synthetic polypeptide
  VARIANT 10: note = Y or S
  VARIANT 14: note = Y or S
  VARIANT 17: note = Y or S
  VARIANT 25: note = Y or S
  VARIANT 27..29: note = Y or S
YYSXSYYXYXYDSXGYXYXYYSXYXYXXX

SEQ ID NO: 938 - protein, 18 aa, synthetic construct
  REGION 1..18: note = Synthetic polypeptide
  VARIANT 2..3: note = Y or S
  VARIANT 5..6: note = Y or S
  VARIANT 8: note = Y or S
  VARIANT 10: note = Y or S
  VARIANT 14: note = Y or S
  VARIANT 16: note = Y or S
  VARIANT 18: note = Y or S
XXXYXXGXGXXYNXXXYX

SEQ ID NO: 939 - protein, 29 aa, synthetic construct
  REGION 1..29: note = Synthetic polypeptide
  VARIANT 1..11: note = Y or S
  VARIANT 16..29: note = Y or S
XXXXXXXXXXXDSSGXXXXXXXXXXXXXX

SEQ ID NO: 940 - protein, 29 aa, synthetic construct
  REGION 1..29: note = synthetic polypeptide
  VARIANT 1..6: note = Y or S
  VARIANT 7: note = Y, S or absent
  VARIANT 8..11: note = Y or S
  VARIANT 16..25: note = Y or S
  VARIANT 26: note = Y, S or absent
  VARIANT 27..29: note = Y or S
XXXXXXXXXXXDSSGXXXXXXXXXXXXXX

SEQ ID NO: 941 - protein, 5 aa, synthetic construct
  REGION 1..5: note = Synthetic polypeptide
ZXZYZ

SEQ ID NO: 942 - protein, 38 aa, synthetic construct
  REGION 1..38: note = Synthetic polypeptide
YYSXSYYXDDDDDDDDDDDDDDDDDDDDDXYYSXYZZZ

SEQ ID NO: 943 - protein, 40 aa, synthetic construct
  REGION 1..40: note = Synthetic polypeptide
ZZZZZZZZZDDDDDDDDDDDDDDDDDDDDZZZZZZZZZZZ